(19) United States
(12) Patent Application Publication
(10) Pub. No.: US 2024/0229163 A1
(43) Pub. Date: Jul. 11, 2024

(54) SEQUENCES FOR DETECTION AND IDENTIFICATION OF METHICILLIN-RESISTANT STAPHYLOCOCCUS AUREUS (MRSA) OF MREJ TYPES xi to xx

(71) Applicant: GENEOHM SCIENCES, INC., San Diego, CA (US)

(72) Inventors: Ann Huletsky, Sillery (CA); Richard Giroux, Sillery (CA)

(21) Appl. No.: 18/528,617

(22) Filed: Dec. 4, 2023

Related U.S. Application Data
(63) Continuation of application No. 11/545,986, filed on Oct. 10, 2006, now Pat. No. 11,834,720.
Application No. 11/545,986 is a continuation-in-part of application No. 11/248,438, filed on Oct. 11, 2005, now Pat. No. 7,838,221.

(51) Int. Cl. C12Q 1/689

(52) U.S. Cl. CPC C12Q 1/689; C12Q 2600/156

(57) ABSTRACT

Described herein are novel SCCmec right extremity junction (MREJ) sequences for the detection and/or identification of methicillin-resistant Staphylococcus aureus (MRSA). Disclosed are methods and compositions based on DNA sequences for the specific detection of MREJ sequences designated types xi, xii, xiii, xiv, xv, xvi, xvii, xviii, xix, and xx for diagnostic purposes and/or epidemiological typing.

### CROSS-REFERENCE TO RELATED APPLICATIONS

This application is a continuation of U.S. patent application Ser. No. 11/545,986, filed Oct. 10, 2006, which is a continuation-in-part of application Ser. No. 11/248,438, filed Oct. 11, 2005, now issued as U.S. Pat. No. 7,838,221.

### REFERENCE TO A SEQUENCE LISTING

The material in the accompanying sequence listing is hereby incorporated by reference into this application. The accompanying sequence listing XML file, named GENOM057P1C1.xml, was created on Dec. 4, 2023 and is 264,281 bytes.

### BACKGROUND OF THE INVENTION

### Field of the Invention

The present invention relates to novel SCCmec right extremity junction sequences for the detection of methicillin-resistant Staphylococcus aureus, and uses thereof for diagnostic and/or epidemiological purposes.

### Description of the Related Art

The coagulase-positive species Staphylococcus aureus is well documented as a human opportunistic pathogen (Murray et al. Eds, 1999, Manual of Clinical Microbiology, 7th Ed., ASM Press, Washington, D.C.). Nosocomial infections caused by S. aureus are a major cause of morbidity and mortality. Some of the most common infections caused by S. aureus involve the skin, and they include furuncles or boils, cellulitis, impetigo, and postoperative wound infections at various sites. Some of the more serious infections produced by S. aureus are bacteremia, pneumonia, osteomyelitis, acute endocarditis, myocarditis, pericarditis, cerebritis, meningitis, scalded skin syndrome, and various abcesses. Food poisoning mediated by staphylococcal enterotoxins is another important syndrome associated with S. aureus. Toxic shock syndrome, a community-acquired disease, has also been attributed to infection or colonization with toxigenic S. aureus.

Methicillin-resistant S. aureus (MRSA) emerged in the 1980s as a major clinical and epidemiologic problem in hospitals (Oliveira et al., 2002, Lancet Infect Dis. 2:180-9). MRSA are resistant to all β-lactams including penicillins, cephalosporins, carbapenems, and monobactams, which are the most commonly used antibiotics to cure S. aureus infections. MRSA infections can only be treated with more toxic and more costly antibiotics, which are normally used as the last line of defense. Since MRSA can spread easily from patient to patient via personnel, hospitals over the world are confronted with the problem to control MRSA. Consequently, there is a need to develop rapid and simple screening or diagnostic tests for detection and/or identification of MRSA to reduce its dissemination and improve the diagnosis and treatment of infected patients.

Methicillin resistance in S. aureus is unique in that it is due to acquisition of DNA from other coagulase-negative staphylococci (CNS), coding for a surnumerary β-lactam-resistant penicillin-binding protein (PBP), which takes over the biosynthetic functions of the normal PBPs when the cell is exposed to β-lactam antibiotics. S. aureus normally contains four PBPs, of which PBPs 1, 2 and 3 are essential. The low-affinity PBP in MRSA, termed PBP 2a (or PBP2′), is encoded by the choromosomal mecA gene and functions as a β-lactam-resistant transpeptidase. The mecA gene is absent from methicillin-sensitive S. aureus but is widely distributed among other species of staphylococci and is highly conserved (Ubukata et al., 1990, Antimicrob. Agents Chemother. 34:170-172).

Nucleotide sequence determination of the DNA region surrounding the mecA gene from S. aureus strain N315 (isolated in Japan in 1982), led to the discovery that the mecA gene is carried by a novel genetic element, designated staphylococcal cassette chromosome mec (SCCmec), which is inserted into the chromosome. SCCmec is a mobile genetic element characterized by the presence of terminal inverted and direct repeats, a set of site-specific recombinase genes (ccrA and ccrB), and the mecA gene complex (Ito et al., 1999, Antimicrob. Agents Chemother. 43:1449-1458; Katayama et al., 2000, Antimicrob. Agents Chemother. 44:1549-1555). SCCmec is precisely excised from the chromosome of S. aureus strain N315 and integrates into a specific S. aureus chromosomal site in the same orientation through the function of a unique set of recombinase genes comprising ccrA and ccrB. Cloning and sequence analysis of the DNA surrounding the mecA gene from MRSA strains NCTC 10442 (the first MRSA strain isolated in England in 1961) and 85/2082 (a strain from New Zealand isolated in 1985) led to the discovery of two novel genetic elements that shared similar structural features of SCCmec. The three SCCmec have been designated type I (NCTC 10442), type II (N315) and type III (85/2082) based on the year of isolation of the strains (Ito et al., 2001, Antimicrob. Agents Chemother. 45:1323-1336). Hiramatsu et al. have found that the SCCmec DNAs are integrated at a specific site in the chromosome of methicillin-sensitive S. aureus (MSSA). The nucleotide sequence of the regions surrounding the left and right boundaries of SCCmec DNA (i.e. attL and attR, respectively), as well as those of the regions around the SCCmec DNA integration site (i.e. attBsce which is the bacterial chromosome attachment site for SCCmec DNA), were analyzed. Sequence analysis of the attL, attR attBsce sites revealed that attBsce is located at the 3′ end of a novel open reading frame (ORF), orfX. orfX encodes a putative 159-amino acid polypeptide that exhibits sequence homology with some previously identified polypeptides of unknown function (Ito et al., 1999, Antimicrob. Agents Chemother. 43:1449-1458). Two new types of SCCmec, designated type IV and type V were recently described (Ma et al., 2002, Antimicrob. Agents Chemother. 46:1147-1152, Ito et al., 2004, Antimicrob Agents Chemother. 48:2637-2651, Oliveira et al., 2001, Microb. Drug Resist. 7:349-360). Sequence analysis of the right extremity of the new SCCmec type IV from S. aureus strains CA05 and 8/6-3P revealed that the sequences were nearly identical over 2000 nucleotides to that of type II SCCmec of S. aureus strain N315 (Ma et al., 2002, Antimicrob. Agents Chemother. 46:1147-1152; Ito et al., 2001, Antimicrob. Agents Chemother. 45:1323-1336). To date, sequence data for the right extremity of the SCCmec type IV from S. aureus strains HDE288 and PL72 is not publicly available (Oliveira et al., 2001, Microb. Drug Resist. 7:349-360).

Methods to detect and identify MRSA based on the detection of the mecA gene and S. aureus-specific chromosomal sequences have been described. (Saito et al., 1995, J. Clin. Microbiol. 33:2498-2500; Ubukata et al., 1992, J. Clin. Microbiol. 30:1728-1733; Murakami et al., 1991, J. Clin. Microbiol. 29:2240-2244; Hiramatsu et al., 1992, Microbiol. Immunol. 36:445-453). However, because the mecA gene is widely distributed in both S. aureus and coagulase-negative staphylococci, these methods are not always capable of discriminating MRSA from methicillin-resistant CNS (Suzuki et al., 1992, Antimicrob. Agents. Chemother. 36:429-434). To address this problem, Hiramatsu et al. developed a PCR-based assay specific for MRSA that utilizes primers that hybridize to the right extremities of the 3 types of SCCmec DNAs in combination with primers specific to the S. aureus chromosome, which corresponds to the nucleotide sequence on the right side of the SCCmec integration site (U.S. Pat. No. 6,156,507, hereinafter the “507 patent”). Nucleotide sequences surrounding the SCCmec integration site in other staphylococcal species (e.g., S. epidermidis and S. haemolyticus) are different from those found in S. aureus. Therefore, this PCR assay is specific for the detection of MRSA.

The PCR assay described in the “507 patent” also led to the development of “MREP typing” (mec right extremity polymorphism) of SCCmec DNA (Ito et al., 2001, Antimicrob. Agents Chemother. 45:1323-1336; Hiramatsu et al., 1996, J. Infect. Chemother. 2:117-129). The MREP typing method takes advantage of the fact that the nucleotide sequences of the three MREJ types differ at the right extremity of SCCmec DNAs adjacent to the integration site among the three types of SCCmec. Compared to type I, type III has a unique nucleotide sequence while type II has an insertion of 102 nucleotides to the right terminus of SCCmec. The MREP typing method described by Hiramatsu et al. uses the following nomenclature: SCCmec type I is MREP type i, SCCmec type II is MREP type ii, and SCCmec type III is MREP type iii.

Because SCCmec types II and IV have the same nucleotide sequence to the right extremity, the MREP typing method described above cannot differentiate the new SCCmec type IV described by Hiramatsu et al. (Ma et al., 2002, Antimicrob. Agents Chemother. 46:1147-1152) from SCCmec type II.

The phrase MREJ refers to the mec right extremity junction «mec right extremity junction». MREJs are approximately 1 kilobase (kb) in length and include sequences from the SCCmec right extremity as well as bacterial chromosomal DNA to the right of the SCCmec integration site. Strains that were classified as MREP types i-iii correspond to MREJ types i-iii. MREJ types iv, v, vi, vii, viii, ix, and x have been previously characterized (Huletsky et al., 2004, J Clin. Microbiol. 42:1875-1884; International Patent Application PCT/CA02/00824).

The embodiments described herein relate to the generation of SCCmec right extremity junction sequence data that enables the detection of more MRSA strains in order to improve NAT assays for detection of MRSA. There is a need for developing more ubiquitous primers and probes for the detection of most MRSA strains around the world.

### SUMMARY OF THE INVENTION

Provided herein are specific, ubiquitous and sensitive methods and compositions for determining the presence and/or amount of nucleic acids from all methicillin-resistant Staphylococcus aureus (MRSA) strains. Methods, compositions and kits are disclosed that enable the detection and quantification of novel MREJ types xi-xx.

Some aspects relate to a method to detect the presence of an MRSA bacterium in a sample comprising bacterial nucleic acids. MRSA strains have SCCmec nucleic acid insert comprising a mecA gene. The SCCmec insert renders the MRSA bacterium resistant to methicillin. The SCCmec is inserted into the bacterial DNA at the 3′ end of the open reading frame orfX, creating a polymorphic right extremity junction (MREJ). At least one primer and/or probe specific for MRSA strains is provided, wherein the primer or probe hybridizes to a polymorphic MREJ nucleic acid of MREJ types xi to xx. The primer(s) and/or probe(s) are annealed with the nucleic acids of the sample. Annealed primer and/or probe indicates the presence of MREJ.

In preferred embodiments, more than one primer and/or probe is provided. The primers and/or probes can anneal to the MREJ nucleic acids under substantially the same annealing conditions. The primers and/or probes can be at least 10 nucleotides, 12 nucleotides, 14 nucleotides, 16 nucleotides, 18 nucleotides, 20 nucleotides, 25 nucleotides, or 30 nucleotides in length. The probes and primers can be used together in the same physical enclosure or in different physical enclosures.

In some embodiments, the primers and/or probes anneal with any one of the nucleic acids of SEQ ID NOs: 15, 16, 17, 18, 19, 20, 21, 25, 26, 39, 40, 41, 42, 55, and 56. In some embodiments, the primers and/or probes altogether can anneal with MREJ types xi to xx, such as SEQ ID NOs: 15, 16, 17, 18, 19, 20, 21, 25, 26, 39, 40, 41, 42, 55, and 56. For example, in some embodiments, the primers and/or probes listed in Table 4 are used to detect MRSA bacteria comprising the following MREJ nucleic acid:

TABLE 4

| Primer/Probe SEQ ID NOs: | To Identify MREJ type |
|---|---|
| 30, 31, 32, 33, 34, 44, 45, 76 | xi |
| 30, 31, 32, 33, 35, 44, 45, 62 | xii |
| 29, 30, 31, 32, 33, 44, 45, 76 | xiii |
| 29, 30, 31, 32, 33, 44, 45, 59 | xiv |
| 24, 30, 31, 32, 33, 4, 45, 62 | xv |
| 36, 44 | xvi |
| 4, 30, 31, 32, 33, 44, 45, 62 | xvii |
| 7, 30, 31, 32, 33, 44, 45, 59 | xviii |
| 9, 30, 31, 32, 33, 44, 45, 59 | xix |
| 8, 30, 31, 32, 33, 44, 45, 59 | xx |

In some embodiments, primers and/or probes are provided that anneal under stringent conditions to more than one MREJ type strain. For example, in preferred embodiments, SEQ ID NOs: 31, 32, 33 are provided for the detection of MREJ types xi to xv and xvii to xx.

In further embodiments primers and/or probes are provided in pairs for the detection of at least one MRSA having MREJ of types xi to xx. Accordingly, in some embodiments, at least one pair of oligonucleotides selected from the group consisting of SEQ ID NOs: 34/45, 34/30, 34/76, and 34/44 are provided for detection of MREJ type xi. In other embodiments, at least one pair of oligonucleotides selected from the group consisting of SEQ ID NOs: 35/45, 35/30, 35/62, and 35/44 are provided for detection of MREJ type xii. In yet other embodiments, at least one pair of oligonucleotides selected from the group consisting of SEQ ID NOs: 29/45, 29/30, 29/76, and 29/44 is provided for detection of MREJ type xiii. In still other embodiments, at least one pair of oligonucleotides selected from the group consisting of SEQ ID NOs: 29/45, 29/30, 29/59, and 29/44 is provided for detection of MREJ type xiv. In other embodiments, at least one pair of oligonucleotides selected from the group consisting of SEQ ID NOs: 24/45, 24/30, 24/62, and 24/44 is provided for detection of MREJ type xv. In yet other embodiments, the oligonucleotides of SEQ ID NOs: 36 and 44 are provided for detection of MREJ type xvi. In still other embodiments, at least one pair of oligonucleotides selected from the group consisting of SEQ ID NOs: 4/45, 4/30, 4/62, and 4/44 is provided for the detection of MREJ type xvii. In yet other embodiments, at least one pair of oligonucleotides selected from the group consisting of 7/45, 7/30, 7/59 and 7/44 is provided for the detection of MREJ type xviii. In other embodiments, at least one pair of oligonucleotides selected from the group consisting of 9/45, 9/30, 9/59 and 9/44 is provided for the detection of MREJ type xix. In yet other embodiments, at least one pair of oligonucleotides selected from the group consisting of SEQ ID NOs: 8/45, 8/30, 8/59, and 8/44 is provided for the detection of MREJ type xx.

In some embodiments, at least two pairs of primers are provided for the detection of more than one MREJ type.

In other preferred embodiments, the primers and/or probes listed in Table 5 are provided together to detect MRSA bacteria comprising the following MREJ nucleic acid:

TABLE 5

| Primer/Probe SEQ ID NOs: | To Identify MREJ type |
|---|---|
| 51, 30, 31, 32, 33 | xi |
| 52, 30, 31, 32, 33 | xii |
| 29, 30, 31, 32, 33 | xiii |
| 29, 30, 31, 32, 33 | xiv |
| 24, 30, 31, 32, 33 | xv |
| 36, 44 | xvi |
| 4, 30, 31, 32, 33 | xvii |
| 7, 30, 31, 32, 33 | xviii |
| 9, 30, 31, 32, 33 | xix |
| 8, 30, 31, 32, 33 | xx |

In further embodiments, the methods described above further comprise providing primers and/or probes specific for a determined MREJ type, and detecting an annealed probe or primer as an indication of the presence of a determined MREJ type.

In yet other embodiments, primers and/or probes specific for the SEQ ID NOs listed in Table 6 are provided to detect MRSA bacteria comprising the following MREJ nucleic acid:

TABLE 6

| Primer/Probe SEQ ID NOs: | To Identify MREJ type |
|---|---|
| 17, 18, 19 | xi |
| 20 | xii |
| 15, 25, 26 | xiii |
| 16 | xiv |
| 56 | xv |
| 21 | xvi |
| 55 | xvii |
| 39, 40 | xviii |
| 41 | xix |
| 42 | xx |

In some embodiments, the primers are used in an amplification reaction, such as polymerase chain reaction (PCR) and variants thereof such as nested PCR and multiplex PCR, ligase chain reaction (LCR), nucleic acid sequence-based amplification (NABSA), self-sustained sequence replication (3SR), strand displacement amplification (SDA), branched DNA signal amplification (bDNA), transcription-mediated amplification (TMA), cycling probe technology (CPT), solid-phase amplification (SPA), nuclease dependent signal amplification (NDSA), rolling circle amplification, anchored strand displacement amplification, solid phase (immobilized) rolling circle amplification, Q beta replicase amplification and other RNA polymerase medicated techniques.

In preferred embodiments, PCR is used to amplify nucleic acids in the sample.

In other embodiments, oligonucleotides of at least 10, 12, 14, 16, 18, 20, 25, or 30 nucleotides in length which hybridize under stringent conditions with any of nucleic acids of SEQ ID NOs: 15, 16, 17, 18, 19, 20, 21, 25, 26, 39, 40, 41, 42, 55, and 56, and which hybridize with one or more MREJ of types selected from xi to xx are also provided.

In other embodiments, primer and/or probe pairs are provided for the detection of MRSA of all of types xi to xx. For example, in certain embodiments, the primer pairs (or probes) listed in Table 7 are provided:

TABLE 7

| Primer/Probe SEQ ID NOs: | To Identify MREJ type: |
|---|---|
| 34/45, 34/30, 34/76, 34/44 | xi |
| 35/45, 35/30, 35/62, 35/44 | xii |
| 29/45, 29/30, 29/76, 29/44 | xiii |
| 29/45, 29/30, 29/59, 29/44 | xiv |
| 24/45, 24/30, 24/62, 24/44 | xv |
| 36/44 | xvi |
| 4/45, 4/30, 4/62, 4/44 | xvii |
| 7/45, 7/30, 7/59, 7/44 | xviii |
| 9/45, 9/30, 9/59, 9/44 | xix |
| 8/45, 8/30, 8/59, 8/44 | xx |

In further embodiments of the method described above, internal probes having nucleotide sequences defined in any one of SEQ ID NOs: 31, 32, and 33 are provided.

In still other embodiments, primers and/or probes used detection of MREJ types xi to xx are used in combination with primers and/or probes capable of detecting MRSA of MREJ types i to x, such as for example those primers and or probes disclosed in co-pending International Patent Application PCT/CA02/00824.

Other aspects of the invention relate to nucleotide sequences comprising at least one of the nucleic acids of SEQ ID NOs: 15, 16, 17, 18, 19, 20, 21, 25, 26, 39, 40, 41, 42, 55, and 56, or the complement thereof. Further embodiments relate to fragments of the nucleic acids of SEQ ID NOs: 15, 16, 17, 18, 19, 20, 21, 25, 26, 39, 40, 41, 42, 55, and 56, wherein the fragments comprise at least 30, 50, 100, 150, 200, 300, or 500 consecutive nucleotides of the nucleic acids of SEQ ID NOs: 15, 16, 17, 18, 19, 20, 21, 25, 26, 39, 40, 41, 42, 55, and 56, or the complements thereof. Further aspects relate to vectors comprising the nucleic acid sequences of SEQ ID NOs: 15, 16, 17, 18, 19, 20, 21, 25, 26, 39, 40, 41, 42, 55, and 56, as well as host cells, such as E. coli host cells, comprising vectors comprising the nucleic acid sequences of SEQ ID NOs: 15, 16, 17, 18, 19, 20, 21, 25, 26, 39, 40, 41, 42, 55, and 56.

Still other aspects relate to oligonucleotides that are at least 10, 12, 14, 16, 18, 20, 25 or 30 nucleotides in length that anneal to any one of SEQ ID NOs: 15, 16, 17, 18, 19, 20, 21, 25, 26, 39, 40, 41, 42, 55, and 56. For example, some embodiments are oligonucleotides that comprise the sequence of any one of SEQ ID NOs: 31, 32, or 33. Yet other embodiments relate to oligonucleotides that are at least 10, 12, 14, 16, 18, 20, 25 or 30 nucleotides in length that anneal to only one of SEQ ID NOs: 15, 16, 17, 18, 19, 20, 21, 25, 26, 39, 40, 41, 42, 55, and 56.

Yet other aspects relate to kits comprising primers and/or probes. The primers and/or probes can be at least 10, 12, 14, 16, 18, 20, 25, or 30 nucleotides in length and hybridize with any one of the nucleic acids of MREJ type xi to xx. Further embodiments relate to kits comprising primers and/or probes that are at least 10, 12, 14, 16, 18, 20, 25, or 30 nucleotides in length and hybridize with any one of the nucleic acids of SEQ ID NOs: 15, 16, 17, 18, 19, 20, 21, 25, 26, 39, 40, 41, 42, 55, and 56. Some embodiments relate to kits that comprise primer pairs. For example, in some embodiments, the kits comprise the following primer pairs:

| Primer/Probe SEQ ID NOs: | To Identify MREJ type: |
|---|---|
| 34/45, 34/30, 34/76, 34/44 | xi |
| 35/45, 35/30, 35/62, 35/44 | xii |
| 29/45, 29/30, 29/76, 29/44 | xiii |
| 29/45, 29/30, 29/59, 29/44 | xiv |
| 24/45, 24/30, 24/62, 24/44 | xv |
| 36/44 | xvi |
| 4/45, 4/30, 4/62, 4/44 | xvii |
| 7/45, 7/30, 7/59, 7/44 | xviii |
| 9/45, 9/30, 9/59, 9/44 | xix |
| 8/45, 8/30, 8/59, 8/44 | xx |

### BRIEF DESCRIPTION OF THE DRAWINGS

FIG. 1 depicts the SCCmec right extremity junctions. Shown are the positions and orientations of the primers used to sequence the novel MREJ types xi to xx. SEQ ID NOs.: 4, 24, 27-30, 36, 43-45, 50-57, 78-86 were used to sequence MREJ types xi, xii, xiii, xiv, xv, xvi, xvii, xviii, xix, and xx. Arrows and numbers below indicate the positions of primers and their respective SEQ ID NOs. Walk indicates the positions where the DNA Walking ACP (DW-ACP) primers from the DNA Walking SpeedUp Kit (Seegene, Del Mar, CA) have annealed on the SCCmec sequence.

FIG. 2 depicts the SCCmec right extremity junction and the position of the primers (SEQ ID NOs.: 4, 7-9, 24, 29-36, 44, 45, 59, 62, 73) developed in the present invention for detection and identification of novel MREJ types xi, xii, xiii, xiv, xv, xvi, xvii, xviii, xix, and xx. Amplicon sizes are listed in Table 11. Numbers in parenthesis under MREJ types indicate MREJ SEQ ID NOs. Arrows indicate the positions of primers and the numbers below indicate their respective SEQ ID NOs. Dark bars and numbers below indicate the positions of probes and their respective SEQ ID NOs. Deletion in MREJ type xvi indicates the position of the 269-bp deletion in orfX.

FIGS. 3A-3Q illustrate a multiple sequence alignment of 19 representative MREJ types i to ix and xi to xx comprising the orfX, the integration site, and the first 535 nucleotides of the SSCmec right extremity. MREJ types i to ix sequences are from co-pending International Patent Application PCT/CA02/00824, and correspond to SEQ ID NO: 89 (mrej_i), SEQ ID NO: 90 (mrej_ii), SEQ ID NO. 91 (mrej_iii), SEQ ID NO: 92 (mrej_iv), SEQ ID NO: 93 (mrej_v), SEQ ID NO: 94 (mrej_vi), SEQ ID NO: 95 (mrej_vii), SEQ ID NO: 96 (mrej_viii), and SEQ ID NO: 97 (mrej_ix) respectively. SEQ ID NO: 18 corresponds to MREJ type xi, SEQ ID NO: 20 corresponds to MREJ type xii, SEQ ID NO: 15 corresponds to MREJ type xiii, SEQ ID NO: 16 corresponds to MREJ type xiv, SEQ ID NO: 56 corresponds to MREJ type xv, SEQ ID NO: 21 corresponds to MREJ type xvi, SEQ ID NO: 55 corresponds to MREJ type xvii, SEQ ID NO: 39 corresponds to MREJ type xviii, SEQ ID NO: 41 corresponds to MREJ type, and SEQ ID NO: 42 corresponds to MREJ type XX.

### DETAILED DESCRIPTION OF THE INVENTION

Methicillin-resistant Staphylococcus aureus (MRSA) pose a serious health threat to individuals and the need for rapid and simple methods for the detection, identification, and quantification of MRSA is readily apparent.

Disclosed herein are novel DNA sequences and DNA arrangements present in MRSA strains that allow for the detection of MRSA that were undetectable using previously available methods. The novel DNA sequences and DNA arrangements are present at the SCCmec region of MRSA DNA. MRSA strains comprise an SCCmec insert that comprises a mecA gene. The SCCmec is inserted into the bacterial DNA at the 3′ end of the orfX open reading frame. The insertion of the SCCmec into the bacterial DNA creates a polymorphic right extremity junction, hereinafter referred to as MREJ standing for «mec right extremity junction». MREJ regions include sequences from the SCCmec right extremity, as well as chromosomal DNA adjacent to the right SCCmec integration site. Embodiments of the invention relate to the novel MREJ sequences and arrangements disclosed herein, which can be used as parental sequences from which primers and/or probes useful in the detection and identification of MRSA described below are derived. Other aspects of the invention relate to novel primers and/or probes derived from the novel MREJ sequences, as well as kits comprising primers and or probes that hybridize to MREJ types xi to xx, for the detection of MRSA.

Also disclosed herein are methods providing for the detection of the presence or absence of an MRSA strain in a sample that includes nucleic acids. At least one primer and/or probe that is specific for MRSA strains and that anneals to an MREJ nucleic acid of types xi to xx, disclosed herein, is provided. The primer(s) and/or probe(s) can be annealed to the nucleic acids of the sample. The detection of annealed primer(s) and/or probe(s) indicates the presence of an MRSA of the MREJ type that hybridizes to the primer(s) and/or probe(s).

### Primers and Probes

As used herein, the terms “primer” and “probe” are not limited to oligonucleotides or nucleic acids, but rather encompass molecules that are analogs of nucleotides, as well as nucleotides. Nucleotides and polynucleotides, as used herein shall be generic to polydeoxyribonucleotides (containing 2-deoxy-D-ribose), to polyribonucleotides (containing D-ribose), to any other type of polynucleotide which is an N- or C-glycoside of a purine or pyrimidine base, and to other polymers containing nonnucleotidic backbones, for example, polyamide (e.g., peptide nucleic acids (PNAs) and polymorpholino (commercially available from the Anti-Virals, Inc., Corvallis, Oreg., as Neugene™ polymers), and other synthetic sequence-specific nucleic acid polymers providing that the polymers contain nucleobases in a configuration which allows for base pairing and base stacking, such as is found in DNA and RNA.

The terms nucleotide and polynucleotide include, for example, 3′-deoxy-2′,5′-DNA, oligodeoxyribonucleotide N3′→P5′ phosphoramidates, 2′-O-alkyl-substituted RNA, double- and single-stranded DNA, as well as double- and single-stranded RNA, DNA:RNA hybrids, and hybrids between PNAs and DNA or RNA. The terms also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine. The resultant modified nucleotide or polynucleotide may form a base pair with other such modified nucleotidic units but not with A, T, C, G or U. For example, guanosine (2-amino-6-oxy-9-beta-D-ribofuranosyl-purine) may be modified to form isoguanosine (2-oxy-6-amino-9-beta-D-ribofuranosyl-purine). Such modification results in a nucleoside base which will no longer effectively form a standard base pair with cytosine. However, modification of cytosine (1-beta-D-ribofuranosyl-2-oxy-4-amino-pyrimidine) to form isocytosine (1-beta-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Mantsch et al. (1975) Biochem. 14:5593-5601, or by the method described U.S. Pat. No. 5,780,610 to Collins et al. The non-natural base pairs referred to as k and x, may be synthesized by the method described in Piccirilli et al. (1990) Nature 343:33-37 for the synthesis of 2,6-diaminopyrimidine and its complement (1-methylpyrazolo[4,3]-pyrimidine-5,7-(4H,6H)-dione. Other such modified nucleotidic units which form unique base pairs have been described in Leach et al. (1992) J. Am. Chem. Soc. 114:3675-3683, or will be apparent to those of ordinary skill in the art.

Primers and/or probes can be provided in any suitable form, included bound to a solid support, liquid, and lyophilized, for example.

Specific binding or annealing of the primers and/or probes to nucleic acid sequences is accomplished through specific hybridization. It will be appreciated by one skilled in the art that specific hybridization is achieved by selecting sequences which are at least substantially complementary to the target or reference nucleic acid sequence. This includes base-pairing of the oligonucleotide target nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under the conditions used to detect the presence of the MRSA nucleic acids.

A positive correlation exists between probe length and both the efficiency and accuracy with which a probe will anneal to a target sequence. In particular, longer sequences have a higher melting temperature (Tₘ) than do shorter ones, and are less likely to be repeated within a given target sequence, thereby minimizing promiscuous hybridization.

As used herein, “Tₘ” and “melting temperature” are interchangeable terms which refer to the temperature at which 50% of a population of double-stranded polynucleotide molecules becomes dissociated into single strands. Formulae for calculating the Tₘ of polynucleotides are well known in the art. For example, the Tₘ may be calculated by the following equation: Tₘ=69.3+0.41 x.(G+C) %-6-50/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2ºC+(number of G+C)×4ºC]. See, e.g., C. R. Newton et al. PCR, 2nd Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically.

Primer or probe sequences with a high G+C content or that comprise palindromic sequences tend to self-hybridize, as do their intended target sites, since unimolecular, rather than bimolecular, hybridization kinetics are generally favored in solution. However, it is also important to design a probe that contains sufficient numbers of G:C nucleotide pairings since each G:C pair is bound by three hydrogen bonds, rather than the two that are found when A and T (or A and U) bases pair to bind the target sequence, and therefore forms a tighter, stronger bond. Preferred G+C content is about 50%.

Hybridization temperature varies inversely with probe annealing efficiency, as does the concentration of organic solvents, e.g., formamide, which might be included in a hybridization mixture, while increases in salt concentration facilitate binding. Under stringent annealing conditions, longer hybridization probes, or synthesis primers, hybridize more efficiently than do shorter ones, which are sufficient under more permissive conditions. Preferably, stringent hybridization is performed in a suitable buffer under conditions that allow the reference or target nucleic acid sequence to hybridize to the probes. Stringent hybridization conditions can vary for example from salt concentrations of less than about 1 M, more usually less than about 500 mM and preferably less than about 200 mM) and hybridization temperatures can range (for example, from as low as 0° C. to greater than 22° C., greater than about 30° C. and (most often) in excess of about 37° C. depending upon the lengths and/or the nucleic acid composition of the probes. Longer fragments may require higher hybridization temperatures for specific hybridization. As several factors affect the stringency of hybridization, the combination of parameters is more important than the absolute measure of a single factor. “Stringent hybridization conditions” refers to either or both of the following: a) 6×SSC at about 45ºC, followed by one or more washes in 0.2×SSC, 0.1% SDS at 65ºC, and b) 400 mM NaCl, 40 mM PIPES pH 6.4, 1 mM EDTA, 50° C. or 70° C. for 12-16 hours, followed by washing.

In the methods described herein, detection of annealed primers and/or probes can be direct or indirect. For example, probes can be annealed to the sample being tested, and detected directly. On the other hand, primers can be annealed to the sample being tested, followed by an amplification step. The amplified products can be detected directly, or through detection of probes that anneal to the amplification products.

In some embodiments, more than one primer and/or probe is provided. For example, some embodiments relate to methods for detecting a plurality of MRSA strains comprising MREJ types xi to xx. A plurality of primers and/or probes may be used in reactions conducted in separate physical enclosures or in the same physical enclosure. Reactions testing for a variety of MRSA types can be conducted one at a time, or simultaneously. In embodiments where the plurality of primers is provided in the same physical enclosure, a multiplex PCR reaction can be conducted, with a plurality of oligonucleotides, most preferably that are all capable of annealing with a target region under common conditions.

In some embodiments, a plurality of primers and/or probes that are specific for different MREJ types are provided in a multiplex PCR reaction, such that the type of the MREJ can be determined. The primers and/or probes used for detection can have different labels, to enable to distinguish one MREJ type from another MREJ type. As used herein, the term “label” refers to entities capable of providing a detectable signal, either directly or indirectly. Exemplary labels include radioisotopes, fluorescent molecules, biotin and the like.

Although the sequences from orfX genes and some SCCmec DNA fragments are available from public databases and have been used to develop DNA-based tests for detection of MRSA, the novel sequence data disclosed herein enable the detection of MRSA of MREJ types xi to xx, which heretofore were not detected using the assays known in the art. These novel sequences, which are listed in Table 8, could not have been predicted nor detected by PCR assays developed based on known MREJ sequences of MRSA (U.S. Pat. No. 6,156,507; International Patent Application PCT/CA02/00824; Ito et al., 2001, Antimicrob. Agents Chemother. 45:1323-1336; Huletsky et al., 2004, J Clin. Microbiol. 42:1875-1884; Ma et al, 2002, Antimicrob. Agents Chemother. 46:1147-1152; Ito et al, Antimicrob Agents Chemother. 2004. 48:2637-2651; Oliveira et al, 2001, Microb. Drug Resist. 7:349-360). Accordingly, the novel MREJ sequences improve current NAT assays for the diagnosis of MRSA as they enable the skilled artisan to design of primers and probes for the detection and/or identification of MRSA strains with MREJ types xi to xx.

Design and Synthesis of Oligonucleotide Primers and/or Probes

All oligonucleotides, including probes for hybridization and primers for DNA amplification, were evaluated for their suitability for hybridization or PCR amplification by computer analysis using publicly and commercially available computer software, such as the Genetics Computer Group GCG Wisconsin package programs, and the Oligo™ 6 and MFOLD 3.0 primer analysis software. The potential suitability of the PCR primer pairs was also evaluated prior to their synthesis by verifying the absence of unwanted features such as long stretches of one nucleotide and a high proportion of G or C residues at the 3′ end (Persing et al., 1993, Diagnostic Molecular Microbiology: Principles and Applications, American Society for Microbiology, Washington, D.C.). Oligonucleotide amplification primers were synthesized using an automated DNA synthesizer (Applied Biosystems).

The oligonucleotide sequence of primers or probes may be derived from either strand of the duplex DNA. The primers or probes may consist of the bases A, G, C, or T or analogs and they may be degenerated at one or more chosen nucleotide position(s), using a nucleotide analog that pairs with any of the four naturally occurring nucleotides. (Nichols et al., 1994, Nature 369:492-493). Primers and probes may also contain nucleotide analogs such as Locked Nucleic Acids (LNA) (Koskin et al., 1998, Tetrahedron 54:3607-3630), and Peptide Nucleic Acids (PNA) (Egholm et al., 1993, Nature 365:566-568). Primers or probes may be of any suitable length, and may be selected anywhere within the DNA sequences from proprietary fragments, or from selected database sequences which are suitable for the detection of MRSA with MREJ types xi to xx. In preferred embodiments, the primers and/or probes are at least 10, 12, 14, 16, 18, 20, 25, or 30 nucleotides in length.

Variants for a given target microbial gene are naturally occurring and are attributable to sequence variation within that gene during evolution (Watson et al., 1987, Molecular Biology of the Gene, 4th ed., The Benjamin/Cummings Publishing Company, Menlo Park, CA; Lewin, 1989, Genes IV, John Wiley & Sons, New York, NY). For example, different strains of the same microbial species may have a single or more nucleotide variation(s) at the oligonucleotide hybridization site. The skilled artisan readily appreciates the existence of variant nucleic acids and/or sequences for a specific gene and that the frequency of sequence variations depends on the selective pressure during evolution on a given gene product. Detection of a variant sequence for a region between two PCR primers may be achieved by sequencing the amplification product. On the other hand, to detect sequence variations that overlap with primer hybridization site, amplification and subsequent sequencing of a larger DNA target with PCR primers outside that hybridization site is required. Similar strategy may be used to detect variations at the hybridization site of a probe. Insofar as the divergence of the target nucleic acids and/or sequences or a part thereof does not affect significantly the sensitivity and/or specificity and/or ubiquity of the amplification primers or probes, variant MREJ sequences are contemplated, as are variant primer and/or probe sequences useful for amplification or hybridization to the variant MREJ.

Oligonucleotide sequences other than those explicitly described herein and which are appropriate for detection and/or identification of MRSA may also be derived from the novel MREJ sequences disclosed herein or selected public database sequences. For example, the oligonucleotide primers or probes may be shorter but of a length of at least 10 nucleotides or longer than the ones chosen; they may also be selected anywhere else in the MREJ sequences disclosed herein or in the sequences selected from public databases. Further, variants of the oligonucleotides disclosed herein can be designed. If the target DNA or a variant thereof hybridizes to a given oligonucleotide, or if the target DNA or a variant thereof can be amplified by a given oligonucleotide PCR primer pair, the converse is also true; a given target DNA may hybridize to a variant oligonucleotide probe or be amplified by a variant oligonucleotide PCR primer. Alternatively, the oligonucleotides may be designed from MREJ sequences for use in amplification methods other than PCR. The primers and/or probes disclosed herein were designed by targeting genomic DNA sequences which are used as a source of specific and ubiquitous oligonucleotide probes and/or amplification primers for MREJ types xi to xx. When a proprietary fragment or a public database sequence is selected for its specificity and ubiquity, it increases the probability that subsets thereof will also be specific and ubiquitous. Accordingly, although the selection and evaluation of oligonucleotides suitable for diagnostic purposes requires much effort, it is quite possible for the individual skilled in the art to derive, from the selected DNA fragments, oligonucleotides other than the ones listed in Tables 9, 10 and 11 which are suitable for diagnostic purposes.

The diagnostic kits, primers and probes disclosed herein can be used to detect and/or identify MRSA of MREJ types xi to xx, in both in vitro and/or in situ applications. For example, it is contemplated that the kits may be used in combination with previously described primers/probes detecting MRSA of MREJ types i to x. It is also contemplated that the diagnostic kits, primers and probes disclosed herein can be used alone or in combination with any other assay suitable to detect and/or identify microorganisms, including but not limited to: any assay based on nucleic acids detection, any immunoassay, any enzymatic assay, any biochemical assay, any lysotypic assay, any serological assay, any differential culture medium, any enrichment culture medium, any selective culture medium, any specific assay medium, any identification culture medium, any enumeration culture medium, any cellular stain, any culture on specific cell lines, and any infectivity assay on animals.

Samples may include but are not limited to: any clinical sample, any environmental sample, any microbial culture, any microbial colony, any tissue, and any cell line.

### DNA Amplification

In some embodiments, an amplification and/or detection step follows the annealing step. Any type of nucleic acid amplification technology can be used in the methods described herein. Non-limiting examples of amplification reactions that can be used in the methods described herein include but are not restricted to: polymerase chain reaction (PCR) (See, PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y. (Innis)), ligase chain reaction (LCR) (See, Wu (1989) Genomics 4:560; Landegren (1988) Science 241:1077; Barringer (1990) Gene 89:117), nucleic acid sequence-based amplification (NASBA), self-sustained sequence replication (3SR)(See, Guatelli (1990) Proc. Natl. Acad. Sci. USA, 87:1874), strand displacement amplification (SDA), branched DNA signal amplification bDNA, transcription-mediated amplification (TMA)(See, Kwoh (1989) Proc. Natl. Acad. Sci. USA 86:1173), cycling probe technology (CPT), nested PCR, multiplex PCR, solid phase amplification (SPA), nuclease dependent signal amplification (NDSA), rolling circle amplification technology (RCA), Anchored strand displacement amplification, solid-phase (immobilized) rolling circle amplification, Q Beta replicase amplification and other RNA polymerase mediated techniques (e.g., NASBA, Cangene, Mississauga, Ontario). These and other techniques are also described in Berger (1987) Methods Enzymol. 152:307-316; Sambrook, Ausubel, Mullis (1987) U.S. Pat. Nos. 4,683,195 and 4,683,202; Amheim (1990) C&EN 36-47; Lomell J. Clin. Chem., 35:1826 (1989); Van Brunt, Biotechnology, 8:291-294 (1990); Wu (1989) Gene 4:560; Sooknanan (1995) Biotechnology 13:563-564.

In preferred embodiments, PCR is used to amplify nucleic acids in the sample. During DNA amplification by PCR, two oligonucleotide primers binding respectively to each strand of the heat-denatured target DNA from the microbial genome are used to amplify exponentially in vitro the target DNA by successive thermal cycles allowing denaturation of the DNA, annealing of the primers and synthesis of new targets at each cycle (Persing et al, 1993, Diagnostic Molecular Microbiology: Principles and Applications, American Society for Microbiology, Washington, D.C.).

Standard amplification protocols may be modified to improve nucleic acid amplification efficiency, including modifications to the reaction mixture. (Chakrabarti and Schutt, 2002, Biotechniques, 32:866-874; Al-Soud and Radstrom, 2002, J. Clin. Microbiol., 38:4463-4470; Al-Soud and Radstrom, 1998, Appl. Environ. Microbiol., 64:3748-3753; Wilson, 1997, Appl. Environ. Microbiol., 63:3741-3751). Such modifications of the amplification reaction mixture include but are not limited to the use of various polymerases or the addition of nucleic acid amplification facilitators such as betaine, BSA, sulfoxides, protein gp32, detergents, cations, and tetramethylamonium chloride.

### Detection of Nucleic Acids

Detection of amplified nucleic acids may include any real-time or post-amplification technologies known to those skilled in the art. Classically, the detection of PCR amplification products is performed by standard ethidium bromide-stained agarose gel electrophoresis, however, the skilled artisan will readily appreciate that other methods for the detection of specific amplification products, which may be faster and more practical for routine diagnosis, may be used, such as those described in co-pending patent application WO01/23604 A2. Amplicon detection may also be performed by solid support or liquid hybridization using species-specific internal DNA probes hybridizing to an amplification product. Such probes may be generated from any sequence from the repertory of MREJ nucleic acids disclosed herein, and designed to specifically hybridize to DNA amplification. Alternatively, amplicons can be characterized by sequencing. See co-pending patent application WO01/23604 A2 for examples of detection and sequencing methods.

Other non-limiting examples of nucleic acid detection technologies that can be used in the embodiments disclosed herein include, but are not limited to the use of fluorescence resonance energy transfer (FRET)-based methods such as adjacent hybridization of probes (including probe-probe and probe-primer methods) (See, J. R. Lakowicz, “Principles of Fluorescence Spectroscopy,” Kluwer Academic/Plenum Publishers, New York, 1999), TaqMan probe technology (See, European Patent EP 0 543 942), molecular beacon probe technology (See, Tyagi et al., (1996) Nat. Biotech. 14:303-308.), Scorpion probe technology (See, Thewell (2000), Nucl. Acids Res. 28:3752), nanoparticle probe technology (See, Elghanian, et al. (1997) Science 277:1078-1081.) and Amplifluor probe technology (See, U.S. Pat. Nos. 5,866,366; 6,090,592; 6,117,635; and 6,117,986).

In preferred embodiments, molecular beacons are used in post-amplification detection of the target nucleic acids. Molecular beacons are single stranded oligonucleotides that, unless bound to target, exist in a hairpin conformation. The 5′ end of the oligonucleotide contains a fluorescent dye. A quencher dye is attached to the 3′ end of the oligonucleotide. When the beacon is not bound to target, the hairpin structure positions the fluorophore and quencher in close proximity, such that no fluorescence can be observed. Once the beacon hybridizes with target, however, the hairpin structure is disrupted, thereby separating the fluorophore and quencher and enabling detection of fluourescence. (See, Kramer FR., 1996, Nat Biotechnol 3:303-8.). Other detection methods include target gene nucleic acids detection via immunological methods, solid phase hybridization methods on filters, chips or any other solid support. In these systems, the hybridization can be monitored by any suitable method known to those skilled in the art, including fluorescence, chemiluminescence, potentiometry, mass spectrometry, plasmon resonance, polarimetry, colorimetry, flow cytometry or scanometry. Nucleotide sequencing, including sequencing by dideoxy termination or sequencing by hybridization (e.g. sequencing using a DNA chip) represents another method to detect and characterize the nucleic acids of target genes.

### MREJ Nucleic Acids

The MREJ fragments disclosed herein were obtained as a repertory of sequences created by amplifying MRSA nucleic acids with novel primers. The amplification and sequencing primers, the repertory of MREJ sequences, and the oligonucleotide sequences derived therefrom for diagnostic purposes, disclosed in Tables 8-11 are further objects of this invention.

Aspects of the invention relate to nucleic acids, in particular nucleic acid sequences from DNA fragments of SCCmec right extremity junction (MREJ), including sequences from SCCmec right extremity and chromosomal DNA to the right of the SCCmec integration site in MRSA types xi to xx. Some embodiments relate to the parental sequences of MREJ types xi to xx from which primers and/or probes specific for the MREJ type xi to xx strain are derived. Thus, some embodiments relate to the nucleotide sequence of SEQ ID NO:15, 16, 17, 18, 19, 20, 21, 25, 26, 39, 40, 41, 42, 55, or 56 or the complement thereof. Other embodiments relate to DNA fragments and oligonucleotides, such as primers and probes. For example, some embodiments relate to nucleic acids comprising at least 10, 20, 30, 40, 50, 75, 100, 150, 200, 250, 300, 350, 400, 450, 500, 600, 700, or 800 consecutive nucleotides of the nucleic acids of SEQ ID NO:15, 16, 17, 18, 19, 20, 21, 25, 26, 39, 40, 41, 42, 55, or 56.

The scope of this invention is not limited to the use of amplification by PCR, but rather includes the use of any nucleic acid amplification method or any other procedure which may be used to increase the sensitivity and/or the rapidity of nucleic acid-based diagnostic tests. The scope of the present invention also covers the use of any nucleic acids amplification and detection technology including real-time or post-amplification detection technologies, any amplification technology combined with detection, any hybridization nucleic acid chips or array technologies, any amplification chips or combination of amplification and hybridization chip technologies. Detection and identification by any nucleotide sequencing method is also under the scope of the present invention.

### Example 1: Evaluation of Previously Described MRSA Diagnostic Amplification Assays

Initially, the literature taught that five types of SCCmec right extremity sequences (SCCmec types I-V) are found among MRSA strains, based on DNA sequence homology (See, Ito et al., 1999, Antimicrob. Agents Chemother. 43:1449-1458; Katayama et al., 2000, Antimicrob. Agents Chemother. 44:1549-1555; Ito et al., 2001, Antimicrob. Agents Chemother. 45:1323-1336; Ma et al., 2002, Antimicrob. Agents Chemother. 46:1147-1152; Ito et al, 2004, Antimicrob. Agents Chemother. 48:2637-2651). SCCmec DNAs are integrated at a specific site of the chromosome of a methicillin-sensitive Staphylococcus aureus (MSSA), named orfX. Generally, each SCCmec type has a unique nucleotide sequence at the right extremity of the SCCmec cassette. The exception to this rule is seen with SCCmec types II and IV, which exhibit nearly identical sequence over 2000 nucleotides. However, SCCmec type II has an insertion of 102 nucleotides to the right terminus of SCCmec type I. Strains classified as SCCmec types I-III fall under the category of MREJ types i-iii.

Recently, we analyzed the MREJ regions of several MRSA strains. We described seven new sequences at the right extremity junction of SCCmec from MRSA that we named MREJ types iv, v, vi, vii, viii, ix, and x (Huletsky et al., 2004, J Clin. Microbiol. 42:1875-1884; International Patent Application PCT/CA02/00824).

We designed a real-time MRSA-specific multiplex PCR assay having primers that target the SCCmec portion of MREJ types i, ii, iii, iv, v, and vii with a primer targeting the S. aureus orfX. Three molecular beacon probes (MBPs) specific to the orfX sequence were used for detection of all sequence polymorphisms identified in this region of the orfX sequence (Huletsky et al., 2004, J. Clin. Microbiol. 42:1875-1884). The oligonucleotide of SEQ ID NO: 30, which hybridizes to the S. aureus orfX, and the oligonucleotides of SEQ ID NOs: 36, 70, 71, 72, and 74, which hybridize to the SCCmec portion of MREJ types i, ii, iii, iv, v, and vii were used in the PCR reaction. Oligonucleotides of SEQ ID NOs: 31, 32, and 33, which hybridize to S. aureus orfX were used as probes. The specificity and ubiquity (i.e., the ability to detect all or most MRSA strains) of the PCR assay was verified using a panel of 569 reference and clinical strains of methicillin-sensitive S. aureus (MSSA) and 1657 different MRSA strains from 32 different countries and which include well-known epidemic clones.

A list of the strains tested and used to build the repertories of MREJ nucleic acids and oligonucleotides derived therefrom disclosed herein is presented in Table 1. The S. aureus clinical isolates used in this invention are part of the SENTRY program collection and several supplier's collections. These S. aureus reference strains or clinical isolates originate from 32 countries: African countries (n=15), Albania (n=2), Argentina (n=50), Australia (n=71), Austria (n=2), Belgium (n=10), Brazil (n=78), Canada (n=607), Chile (n=42), China (n=70), Denmark (n=33), Egypt (n=1), Finland (n=12), France (n=50), Germany (n=47), Greece (n=7), Ireland (n=5), Israel (n=19), Italy (n=61), Japan (n=62), Mexico (n=1), The Netherlands (n=179), Poland (n=33), Portugal (n=24), Singapore (n=20), Slovenia (n=12), Spain (n=31), Sweden (n=10), Switzerland (n=13), Turkey (n=28), United Kingdom (n=22), and United States (n=528). Confirmation of the identification of the staphylococcal strains was performed by using the MicroScan WalkAway Panel type Positive Breakpoint Combo 13 when required (Dade Behring Canada Inc., Mississauga, Ontario, Canada). When needed, the identity was reconfirmed by PCR analysis using S. aureus-specific primers and mecA-specific primers (SEQ ID NOs.: 50, 60, 61, 63) (Martincau et al., 2000, Antimicrob. Agents Chemother. 44:231-238). The data from the assay is presented in Table 2.

Among the 569 MSSA strains tested, 26 strains were misidentified as MRSA based on the PCR assay. Of the 1657 MRSA strains tested, 1640 were specifically detected with the PCR assay whereas 23 of these MRSA strains, representing a broad variety of origins were not detected by the assay. Thus, the specificity and ubiquity (i.e. the ability to detect all or most MRSA strains) of this PCR assay was verified. Four of these 23 MRSA strains, CCRI-9208, CCRI-9770, CCRI-9681, and CCRI-9860, which were not detected in the above assay have previously been shown to harbor the MREJ types vi, viii, ix, and x, respectively (International Patent Application PCT/CA02/00824).

The 19 remaining MRSA strains that were not detected in the assay were analyzed further. PCR was performed on the genomic DNA from each strain, using a primer targeting the sequence at the SCCmec right extremity of MREJ types vi, viii, or ix in combination with a primer targeting the S. aureus orfX. Specifically, each PCR reaction contained the oligonucleotide of SEQ ID NO:65, which anneals to MREJ type vi, the oligonucleotide of SEQ ID NO:75, which anneals to MREJ type viii, or the oligonucleotide of SEQ ID NO:29, which anneals to MREJ type ix, in combination with the oligonucleotide of SEQ ID NO:30, which is a S. aureus-specific primer. MREJ type x was previously shown to have a deletion of the complete orfX and a portion at the right extremity of SCCmec type II (International Patent Application PCT/CA02/00824). Therefore, the oligonucleotide of SEQ ID NO:77, which anneals to orf22 in the S. aureus chromosome, and the oligonucleotide of SEQ ID NO:73, which anneals to orf27 located in SCCmec type II were used in a PCR reaction to detect MREJ type x. Two out of 19 strains, CCRI-11879 and CCRI-12036, were shown to harbor MREJ type ix with these PCR primers. However, 17 MRSA strains were not detected with primers targeting MREJ types vi, viii, ix, and x suggesting that these strains harbor new MREJ types (Tables 2 and 3).

### Example 2: Sequencing of Novel MREJ Types from MRSA

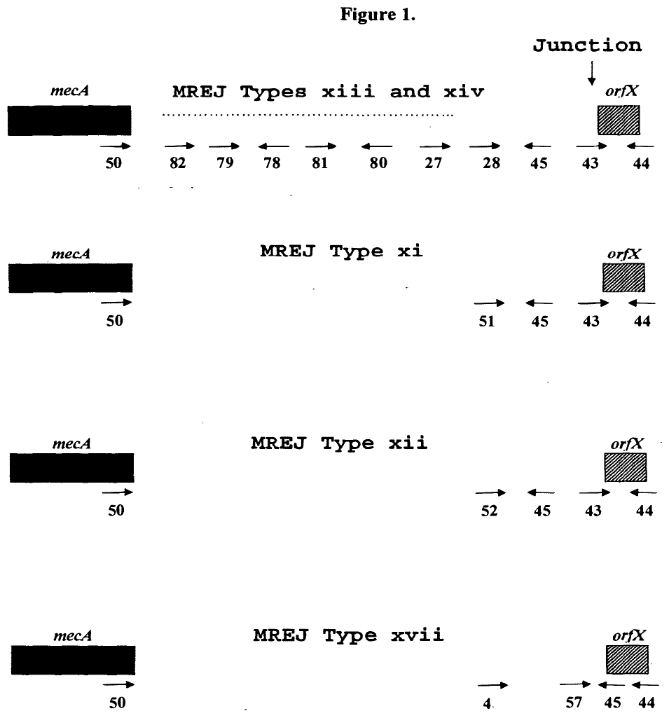

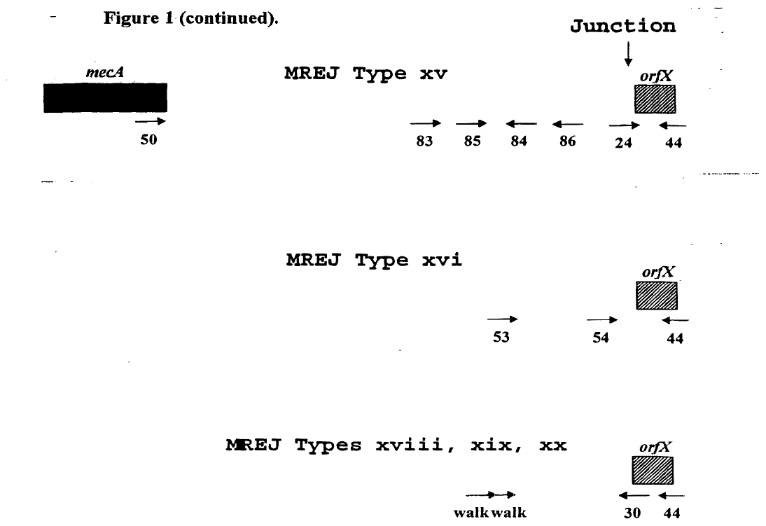

To further characterize the MREJ region of the 17 MRSA strains from which DNA was not amplified with primers that allow the detection of MREJ types i to x, the nucleotide sequence of MREJ for 15 of these 17 MRSA strains was determined. First, a primer that anneals to mecA (SEQ ID NO.: 50) and a primer that anneals to the 5′ end of orfX (SEQ ID NO.: 44) were used together in a PCR reaction to amplify MREJ fragments of MRSA. The strategy used to select these primers is illustrated in FIG. 1. Four identical PCR reactions, each containing 100 ng of purified genomic DNA were performed. Each PCR reaction contained 1X HERCULASE™ DNA polymerase buffer (Stratagene, La Jolla, CA), 0.8 μM of each of the oligos of SEQ ID NOs.: 44 and 50, 0.56 mM of each of the four dNTPs and 5 units of HERCULASE™ DNA polymerase (Stratagene, La Jolla, CA) with 1 mM MgCl₂ in a final volume of 50 μl. PCR reactions were subjected to cycling using a standard thermal cycler (PTC-200 from MJ Research Inc.) as follows: 2 min at 92° C. followed by 35 or 40 cycles of 10 see at 92° C. for the denaturation step, 30 see at 55° C. for the annealing step and 15 min at 68° C. for the extension step.

The four PCR reactions were pooled. 10 μL of the PCR reaction was resolved by electrophoresis in a 0.7% agarose gel containing 0.25 μg/mL of ethidium bromide. The amplicons were then visualized with an Alpha-Imager (Alpha Innotech Corporation, San Leandro, CA) by exposing to UV light at 254 nm. The remaining PCR-amplified mixture (150-200 μl, total) was also resolved by electrophoresis in a 0.7% agarose gel and visualized by staining with methylene blue (Flores et al., 1992, Biotechniques, 13:203-205).

Of the 15 strains tested, the following eight yielded amplification products ranging from 12-20 kb in length with SEQ ID NOs.: 44 and 50 as primers: CCRI-11976, CCRI-11999, CCRI-12157, CCRI-12198, CCRI-12199, CCRI-12719, CCRI-9887, CCRI-9772. The amplification products were excised from the agarose gel and purified using the QIAquick™ gel extraction kit (QIAGEN Inc., Valencia, CA). The gel-purified DNA fragments were used directly in sequencing reactions. Both strands of the MREJ amplification products were sequenced by the dideoxynucleotide chain termination sequencing method using an Applied Biosystems automated DNA sequencer (model 377 or 3730×1) with their Big Dye™ Terminator Cycle Sequencing Ready Reaction Kit (Applied Biosystems, Foster City, CA). 425-495 ng of the gel-purified amplicons were used in sequencing reactions with SEQ ID NO.: 44, which was used for the amplification reaction. Based on the sequence information generated from the reactions with SEQ ID NO:44, internal sequencing primers were designed and used to obtain sequence data from both strands for a larger portion of each amplicon preparation. Specifically, the oligonucleotides of SEQ ID NOs.: 43 and 45 were used to sequence MRSA strains CCRI-11976 and CCRI-11999; SEQ ID NOs.: 43, 45, and 51 were used to sequence MRSA strains CCRI-12157, CCRI-12198, and CCRI-12199; SEQ ID NOs.: 43, 45, and 52 were used to sequence MRSA strain CCRI-12719; SEQ ID NO.: 24 was used to sequence MRSA strain CCRI-9887, and SEQ ID NOs.: 4, 45, and 57 were used to sequence MRSA strain CCRI-9772 (FIG. 1, Tables 9 and 11). The sequences of the 8 strains described in Table 3 are presented as SEQ ID NOs.: 15, 16, 17, 18, 19, 20, 55, and 56 (Table 8).

To ensure that the determined sequence did not contain errors attributable to the sequencing of PCR artifacts, two independent preparations of the gel-purified MREJ amplification products originating from two independent PCR amplifications were sequenced as described above. For most target fragments, the sequences determined for both amplicon preparations were identical. Furthermore, the sequences of both strands were 100% complementary thereby confirming the high accuracy of the determined sequence. The MREJ sequences determined using the above strategy are described in the Sequence Listing and in Table 8.

A different set of oligonucleotide primers (described in Oliviera et. al.) was used to further analyze the 17 MRSA strains that did not yield amplification products with primers for detection of MREJ types i-vii (Oliveira and de Lencastre. 2002, Antimicrob. Agents Chemother. 46:2155-2161). Two strains, (CCRI-12382 and CCRI-12383), harbored SCCmec type III and contained sequences specific to the ψccr complex. Another strain, (CCRI-12845), harbors SCCmec type II.

To determine the MREJ sequences of strains CCRI-12382 and CCRI-12383, a primer targeting the ψccr complex sequence located in SCCmec type III (SEQ ID NO.: 27) was used in combination with a primer targeting the 5′end of orfX (SEQ ID NO.: 44) to amplify MREJ fragments of these two MRSA strains (Table 10 and FIG. 1). Four identical PCR reactions, each containing 100 ng of purified genomic DNA were performed. Each PCR reaction contained 1X HERCULASE™ DNA polymerase buffer (Stratagene, La Jolla, CA), 0.8 μM of each of the 2 primers (SEQ ID NOs.: 27 and 44), 0.56 mM of each of the four dNTPs and 5 units of HERCULASE™ DNA polymerase (Stratagene, La Jolla, CA) with 1 mM MgCl₂ in a final volume of 50 μl. The PCR reactions were cycled using a standard thermal cycler (PTC-200 from MJ Research Inc., Watertown, MA) as follows: 2 min at 92° C. followed by 35 cycles of 10 see at 92° C. for the denaturation step, 30 see at 55° C. for the annealing step and 15 min at 68° C. for the extension step.

The PCR reactions were pooled and 10 μl of the PCR-amplified mixture was resolved by electrophoresis in a 0.7% agarose gel containing 0.25 μg/ml of ethidium bromide. The amplicons were then visualized with an Alpha-Imager (Alpha Innotech Corporation, San Leandro, CA) by exposing to UV light at 254 nm. The remaining PCR-amplified mixture (150-200 μl, total) was also resolved by electrophoresis in a 0.7% agarose gel and visualized by staining with methylene blue as described above. For these two MRSA strains, an amplification product of ˜ 8 kb was obtained. The PCR amplification products were excised from the agarose gel and purified as described above. The gel-purified DNA fragment was then used directly in the sequencing protocol as described above. The sequencing reactions were performed by using SEQ ID NO.: 44 (also used in the amplification reaction) and 425-495 ng of the gel-purified amplicons for each reaction. Subsequently, different sets of internal sequencing primers were used to obtain sequence data from both strands and for a larger portion of the amplicon (SEQ ID NOs.: 28, 30, and 43) (FIG. 1, Tables 9 and 11). The sequence of the MRSA strains CCRI-12382 and CCRI-12383 described in Table 3 which were sequenced using this strategy are designated SEQ ID NOs.: 25 and 26, respectively (Table 8).

To sequence the MREJ fragment of strain CCRI-12845 (SCCmec type II) PCR amplification was performed using the oligonucleotide of SEQ ID NO:44, which anneals to the 5′ end of orfX in combination with the oligonucleotide of SEQ ID NO:53, which anneals to the SCCmec right extremity of MREJ type ii. 1 μL of a purified genomic DNA preparation was transferred directly into 4 tubes containing 39 μL of a PCR reaction mixture. Each PCR reaction contained 50 mM KCl, 10 mM Tris-HCl (pH 9.0), 0.1% Triton X-100, 2.5 mM MgCl₂, 0.4 μM of each of the oligonucleotides of SEQ ID NO.: 44 and 53, 200 μM of each of the four dNTPs, 3.3 □g/□l of BSA (Sigma-Aldrich Canada Ltd) and 0.5 unit of Taq DNA polymerase (Promega, Madison, WI) coupled with the TaqStart™ Antibody (BD Bisociences, San Jose, CA). PCR reactions were performed using a standard thermocycler (PTC-200 from MJ Research Inc., Watertown, MA) as follows: 3 min at 94° C. followed by 40 cycles of 5 see at 95° C. for the denaturation step, 1 min at 58° C. for the annealing step and 1 min at 72° C. for the extension step. An amplification product of 4.5 kb was obtained with this primer set.

The amplification products were pooled and 10 μl of the mixture were resolved by electrophoresis in a 1.2% agarose gel containing 0.25 μg/ml of ethidium bromide. The amplicons were then visualized with the Alpha-Imager. Amplicon size was estimated by comparison with a 1 kb molecular weight ladder (Life Technologies, Bethesda, MD). The remaining PCR-amplified mixture (150 μl, total) was also resolved by electrophoresis in a 1.2% agarose gel and visualized by staining with methylene blue as described above. The PCR reaction yielded a 1.2 kb amplification product. The band corresponding to this specific amplification product was excised from the agarose gel and purified as described above. The gel-purified DNA fragment was then used directly in the sequencing protocol as described above. The sequencing reactions were performed using the oligonucleotides of SEQ ID NOs.: 44 and 53 as well as one internal primer (SEQ ID NO.: 54) and 10 ng/100 bp per reaction of the gel-purified amplicons (FIG. 1, Table 10). The MREJ sequence of strain CCRI-12845 is designated as SEQ ID NO.: 21 (Table 8).

To determine the MREJ sequences of the 4 last MRSA strains (CCRI-12524, CCRI-12535, CCRI-12810, and CCRI-12905), the oligonucleotide of SEQ ID NO: 44 was used in combination with each of the four DNA Walking ACP (DW-ACP) primers from the DNA WALKING SPEED UP™ Sequencing Kit (Seegene, Del Mar, CA) according to the manufacturer's instructions on a PTC-200 thermocycler. The DW-ACP primer system (DW ACP-PCR™ Technology) enables one to obtain genuine unknown target amplification products up to 2 kb. A first amplification product obtained with one of the DW-ACP primers was purified using the QIAQUIK™ PCR purification Kit (QIAGEN Inc., Valencia, CA). The purified PCR product was re-amplified using the DW-ACP-N primer in combination with the oligonucleotide of SEQ ID NO:30, which anneals to orfX under manufacturer recommended PCR conditions. The PCR-amplified mixture of 4 different 50-μL PCR reactions were pooled and resolved by electrophoresis in a 1.2% agarose gel. The amplicons were then visualized by staining with methylene blue as described above. Amplicon size was once again estimated by comparison with a 1 kb molecular weight ladder. An amplification product of 1.5 to 3 kb was obtained. The amplification product was excised from the agarose gel and purified as described above and the DNA was then used directly in the sequencing protocol as described above. 10 ng of purified DNA for every 100 bp of the amplicon was used in sequencing reactions using the oligonucleotides of SEQ ID NO.: 30 and DW-ACP-N. The MREJ sequences from MRSA strains strains CCRI-12524, CCRI-12535, CCRI-12810, and CCRI-12905 (described in Table 3) are designated SEQ ID NOs.: 39, 40, 41, and 42 (Table 8).

CCRI-12376 and CCRI-12593 described in Table 3 were not sequenced but rather characterized using PCR primers and shown to contain MREJ type xiii using specific amplification primers.

### Example 3: Sequence Analysis of Novel MREJ Types xi-xx

The sequences obtained for 15 of the 17 strains non-amplifiable by the MRSA-specific primers detecting MREJ types i to x previously described were compared to the sequences available from public databases. In all cases except MRSA strain CCRI-12845, the orfX portion of the MREJ sequence had an identity close to 100% to publicly available sequences for orfX. CCRI-12845 has a deletion in orfX (SEQ ID NO.: 21) (described below). While the orfX portion of most MREJ fragments (SEQ ID NOs.: 15-20, 25-26, 39-42, 55-56) shared nearly 100% identity with publicly available S. aureus orfX sequences, with the exception of strain CCRI-12845, the DNA sequence within the right extremity of SCCmec itself was shown to be different from those of MREJ types i, ii, iii, iv, v, vi, vii, viii, ix, and x (International Patent Application PCT/CA02/00824; U.S. Pat. No. 6,156,507). The DNA sequence within the right extremity of SCCmec of CCRI-12845 was similar to that of MREJ type ii (see below). Thus, ten different novel MREJ sequence types are reported herein: MREJ types xi to xx.

The sequences within the right extremity of SCCmec obtained from strains CCRI-12157, CCRI-12198, and CCRI-12199 (SEQ ID NOs.: 17, 18, and 19) were nearly identical to each other, and different from those of MREJ types i, ii, iii, iv, v, vi, vii, viii, ix, and x (Ito et al., 2001, Antimicrob. Agents Chemother. 45:1323-1336; Ma et al., 2002, Antimicrob. Agents Chemother. 46:1147-1152, Huletsky et al., 2004, J. Clin. Microbiol. 42:1875-1884, International Patent Application PCT/CA02/00824, U.S. Pat. No. 6,156,507). These new sequences were designated as MREJ type xi (SEQ ID NOs.: 17-19). A BLAST™ search revealed that the first 86 bp of the SCCmec portion of MREJ type xi exhibited 87% identity with an unknown sequence of Staphylococcus epidermidis strain SRI (GenBank accession number AF270046). The remainder of the MREJ sequence was shown to be unique, exhibiting no significant homology to any published sequence.

The sequence obtained at the right extremity of SCCmec from strain CCRI-12719 (SEQ ID NO.: 20) was different from MREJ types i to x as well as from MREJ type xi. The new MREJ type was designated as MREJ type xii. When compared with GenBank sequences using BLAST™, the sequence at the right extremity of SCCmec of MREJ type xii exhibited 100% identity with the sequence found at the right extremity of the SCCmec type V recently described (Ito et al., 2004, Antimicrob. Agents. Chemother. 48:2637-2651; GenBank accession number AB121219). The sequence also exhibited 85% identity with a 212-nucleotide region of the Staphylococcus epidermidis RP62a putative GTP-binding protein sequence.

The sequences within the right extremity of SCCmec obtained from strains CCRI-11976, CCRI-12382, and CCRI-12383 (SEQ ID NOs.: 15, 25, and 26) were 100% identical to each other, different from MREJ types i to x as well as from MREJ types xi and xii. The new MREJ sequences were designated as MREJ type xiii (SEQ ID NOs.: 15, 25, and 26).

The sequence within the right extremity of SCCmec obtained from strain CCRI-11999 (SEQ ID NO.: 16) was also different from MREJ types i to x as well as from MREJ types xi, xii, and xiii, and consequently, was designated as MREJ type xiv. A BLAST™ search of the MREJ types xiii and xiv sequences showed that a portion of the SCCmec of these two MREJ types was identical to that of MREJ type ix. Indeed, the SCCmec portions of MREJ types ix and xiv were preceded by one and two consecutive 102 bp insertions, respectively, when compared to MREJ type xiii. The rest of the MREJ types ix, xiii, and xiv sequences were 99.9% identical to each other. These sequences exhibited identities ranging from 97% to 100% (for the highest BLAST scores) with non-contiguous regions (in varying sizes of 1535 to 1880 nucleotides) of the SCC cassette without mecA harboring the chromosome recombinase genes of the methicillin-susceptible strain S. epidermidis ATCC 12228 (GenBank accession number BK001539). The sequence of the 102-pb insertion was 99-100% identical to that found in MREJ type ii.

The sequence obtained within the right extremity of SCCmec from strain CCRI-9887 was different from MREJ types i to x as well as from MREJ types xi to xiv and was therefore designated as MREJ type xv (SEQ ID NO.: 56). A BLAST search of the sequence obtained within the SCCmec portion of MREJ type xv revealed that this DNA fragment exhibited identities ranging from 92% to 96% (for the highest BLAST scores) with non-contiguous sequences (in varying sizes of 342 to 618 nucleotides) of the SCC cassette (which do not contain mecA) of the methicillin-susceptible S. aureus strain M (GenBank accession number U10927). Although the sequence of MREJ type xv has been described, the localization of this sequence downstream of orfX in a MRSA strain has heretofore not been described. The CCRI-9887 MREJ sequence also exhibited 94% identity with a 306-nucleotide region of strain Staphylococcus haemolyticus JCSC1435 located near the orfX sequence.

The sequence obtained for MREJ from strain CCRI-12845 (SEQ ID NO.: 21) revealed that the MREJ fragment of this strain has a deletion of nucleotides 165 to 434 of orfX (269-bp fragment), whereas the sequence at the right extremity of SCCmec (328 nucleotides) had identities ranging from 99.8 to 100% with that of MREJ type ii available in public databases. Although the MREJ sequence obtained from this strain exhibited a high level of identity with known MREJ sequences, the presence of a 269-bp deletion within orfX had heretofore never been described. As one of the oligonucleotides used in the initial PCR amplification assay described above falls within this 269 bp deletion, the deletion in orfX explains why this MRSA strain was not or could not have been detected with primers and probes previously described to detect MRSA (U.S. Pat. No. 6,156,507 and International Patent Application PCT/CA02/00824). The novel MREJ sequence of this strain was designated as MREJ type xvi.

The sequence obtained at the right extremity of SCCmec from strain CCRI-9772 was different from MREJ types i to x as well as from MREJ types xi to xvi. The new MREJ type was designated as MREJ type xvii (SEQ ID NO.: 55). A BLAST™ search against the GenBank database revealed that the SCCmec portion of MREJ type xvii sequence exhibited 100% identity with the sequence at left of the SCCmec junction of S. aureus strain CA05 (JCSC 1968) (GenBank Accession number AB063172) harbouring SCCmec type IV (Ma et al., 2002. Antimicrob. Agents Chemother. 46:1147-1152). The genetic organization of MREJ type xvii is similar to the region downstream of orfx in MSSA. Although the sequence itself has been described previously, the localization of this sequence downstream of orfX in a MRSA strain has heretofore never been described.

The sequences obtained from the right extremity of SCCmec from strains CCRI-12524 and CCRI-12535 were nearly identical to each other but were different from MREJ types i to x as well as from MREJ types xi to xvii and were therefore designated as MREJ type xviii (SEQ ID NOs.: 39 and 40). A BLAST search against GenBank sequences revealed a 100% identity with a 487-nucleotide region of the SCCmec cassette of Staphylococcus haemolyticus JCSC 1435. The remainder of the sequence was shown to be unique, exhibiting no significant homology to any published sequence.

The sequence obtained from strain CCRI-12810 was different from MREJ types i to x as well as from MREJ types xi to xviii and was designated as MREJ type xix (SEQ ID NO.: 41). When compared with GenBank sequences using BLAST, the SCCmec portion of MREJ type xix sequence exhibited 100% identity with a 597-nucleotide region of unknown function of strain ATCC 25923 which is located at the left of SCCmec (GenBank accession number AB047239). This result has been observed with four other MRSA strains for which the SCCmec sequences have been published: MRSA252, 85/3907, 85/2082, and MR108 (GenBank accession numbers: BX571856, AB047088, AB037671 and AB096217, respectively). The genetic organization of MREJ type xix is similar to the region downstream of orfx in MSSA. Although the sequence itself had been described, the presence of this DNA fragment downstream of orfX had heretofore never been described.

The sequence obtained at the right extremity of SCCmec from strain CCRI-12905 was different from MREJ types i to x as well as from MREJ types xi to xix and was designated as MREJ type xx (SEQ ID NO.: 42). When compared with Genbank sequences using BLAST, the SCCmec of MREJ type xx sequence exhibited 100% and 99% identities with two non-contiguous sequences (respectively 727 and 307 nucleotides long) downstream of orfX of the methicillin-susceptible S. aureus strain NCTC 8325 (GenBank accession number AB014440). The genetic organization of MREJ type xx is similar to the region downstream of orfx in MSSA. The localization of this sequence downstream of orfX in a MRSA strain has heretofore never been described. Identity levels ranging from 98% to 100% with non-contiguous fragments (in varying sizes of 91 to 727 nucleotides) was found with 11 MRSA strains for which the SCCmec sequences have been published: N315, NCTC 10442, COL, USA300, Mu50, 2314, 85/4231, 85/2235, JCSC 1978, PL72, HDE 288 (GenBank accession numbers: BA000018, AB033763, CP000046, CP000255, BA000017, AY271717, AB014428, AB014427, AB063173, AF411936, AF411935, respectively). These identical fragments are located downstream of the mecA gene towards (or even downstream) the left insertion point of SCCmec.

### Example 4: Sequence Comparison of New MREJ Types xi to xx

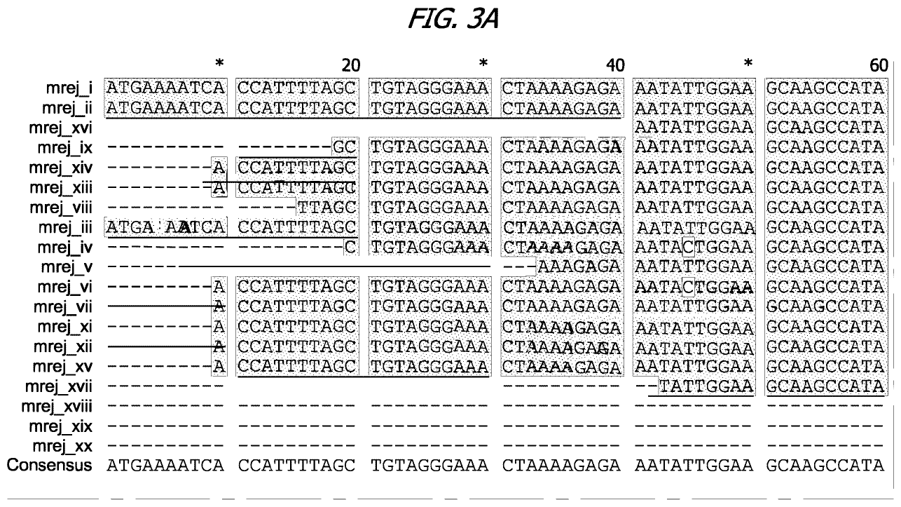

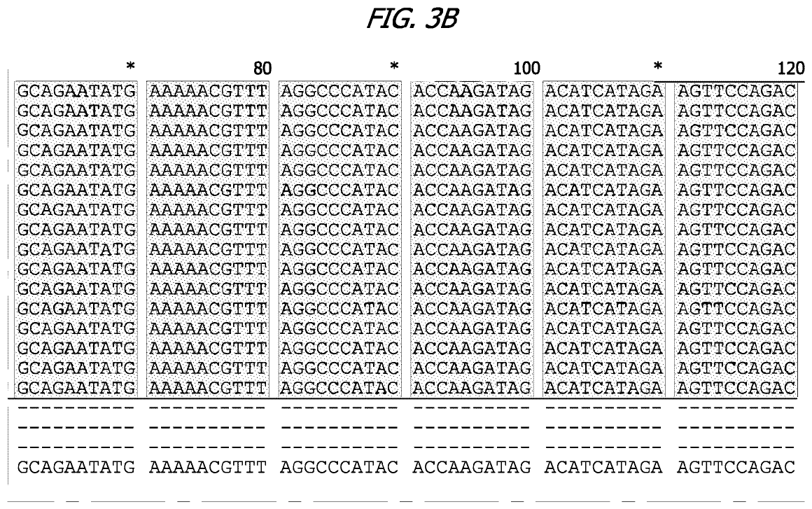

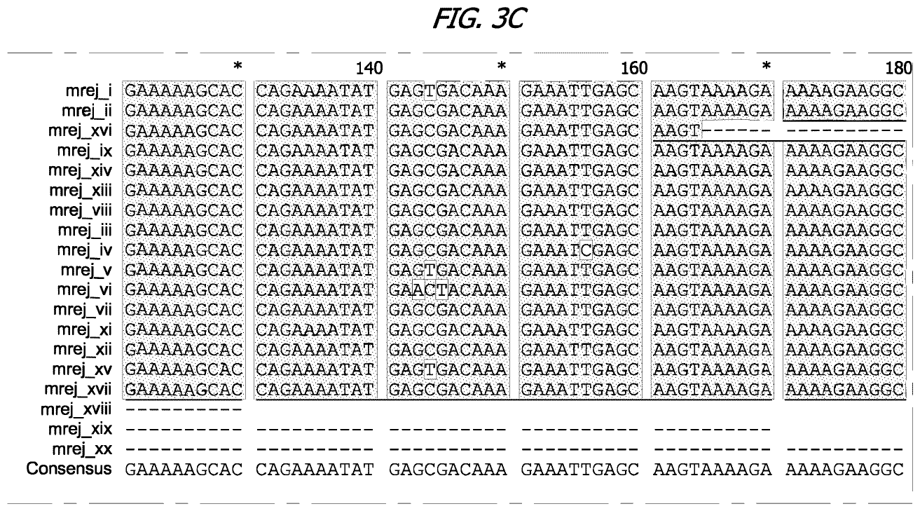

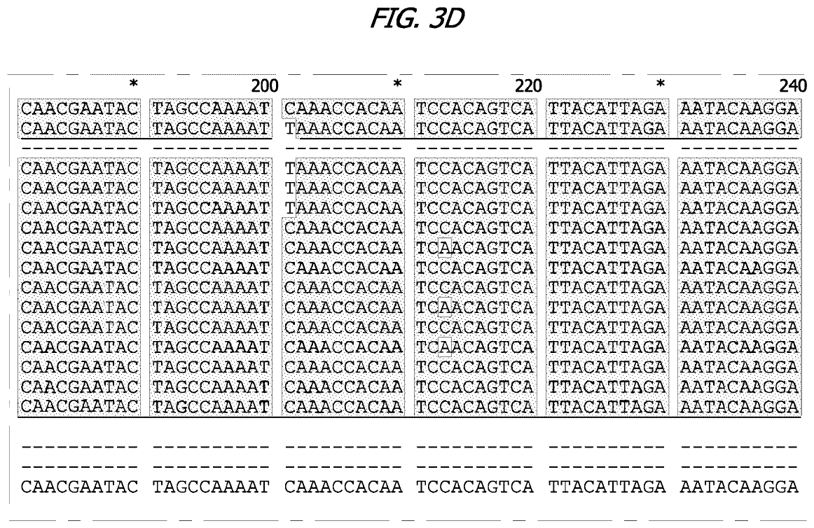

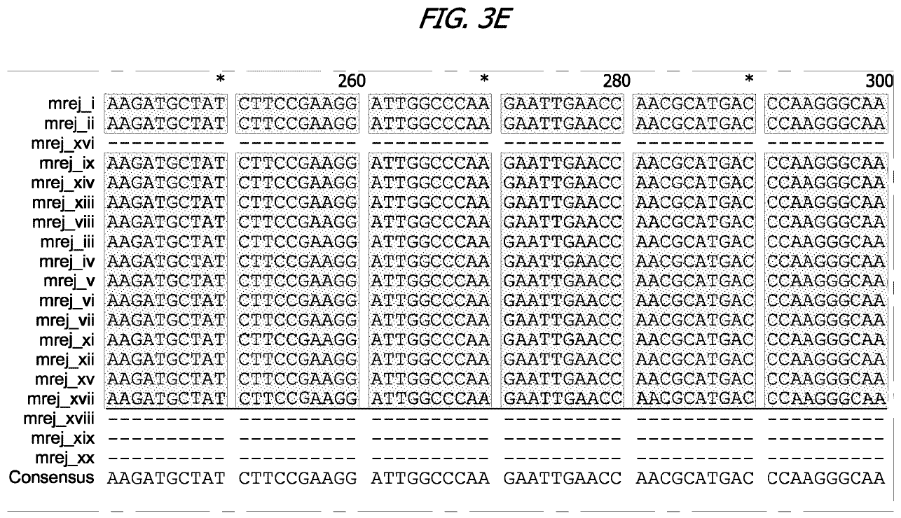

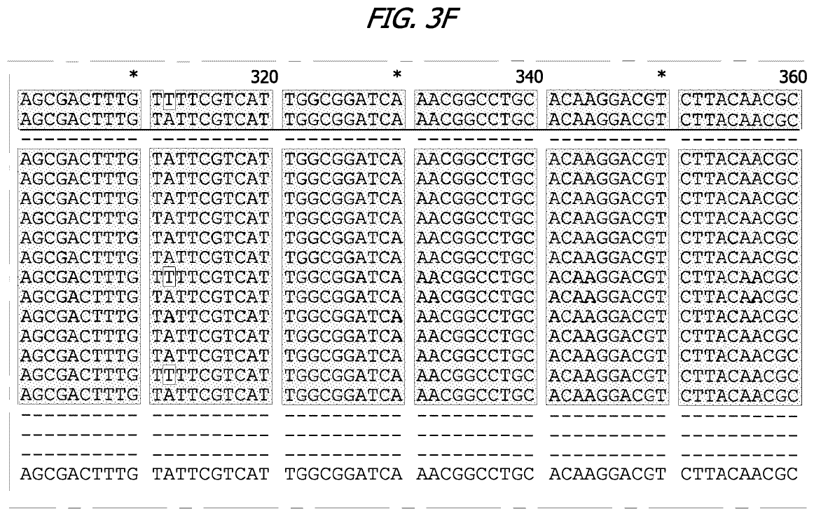

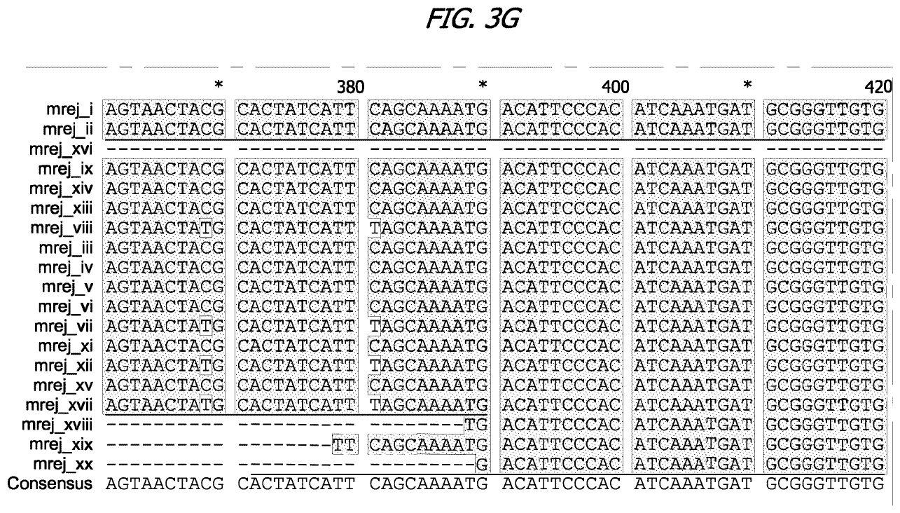

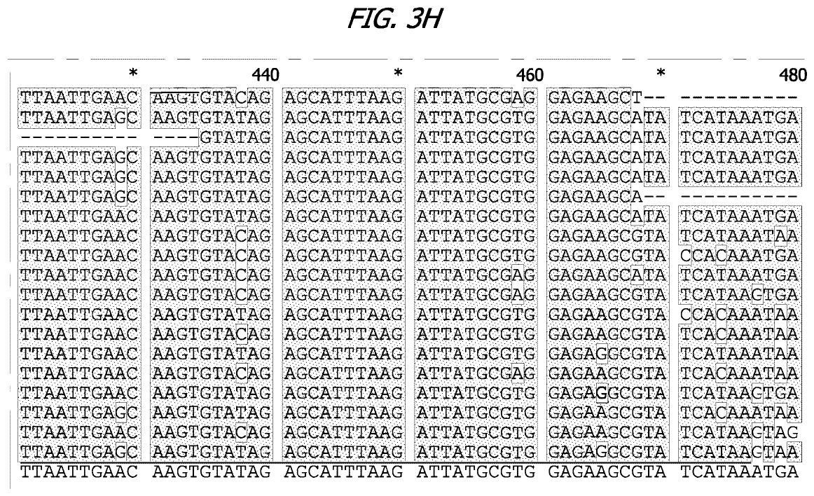

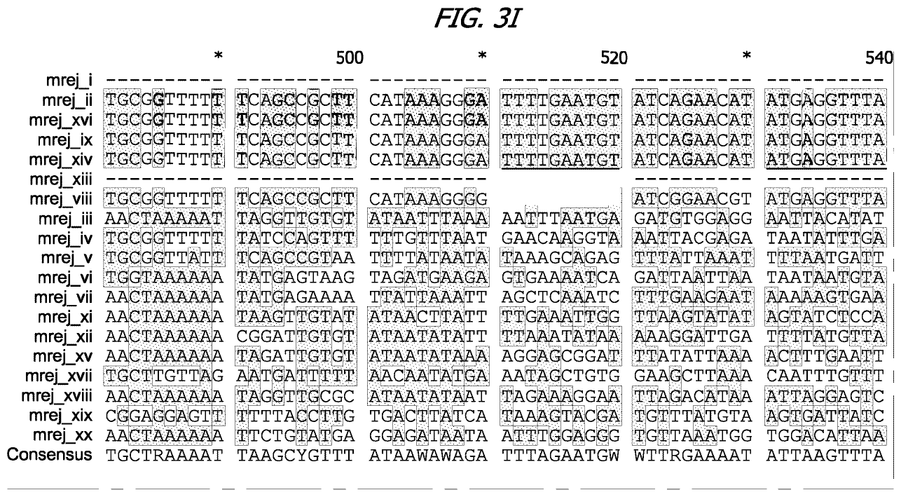

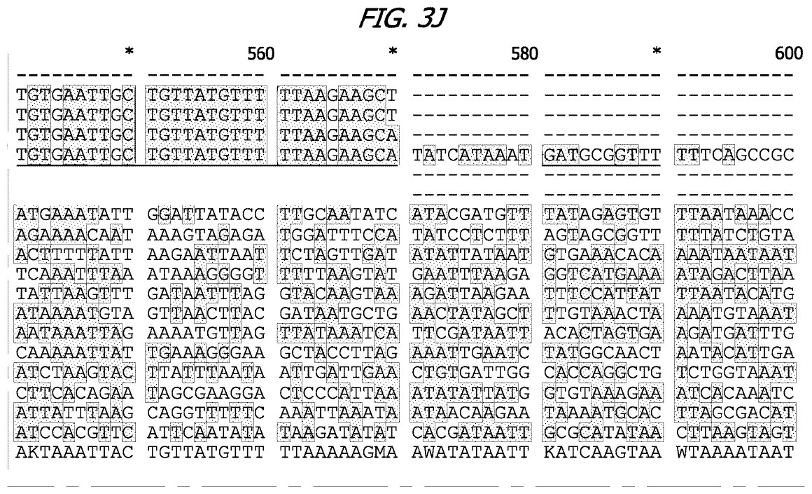

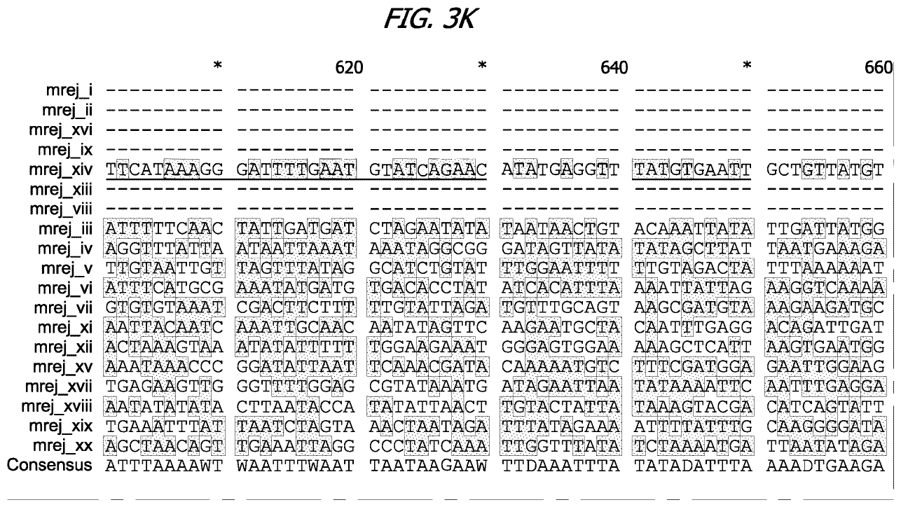

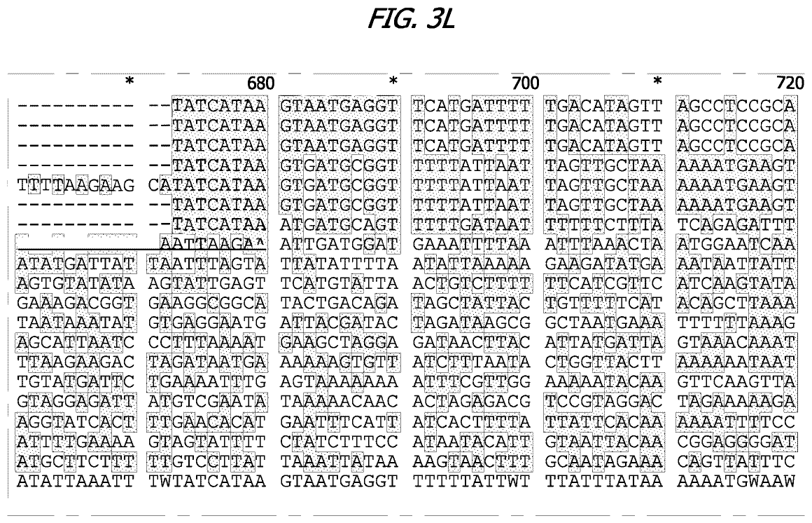

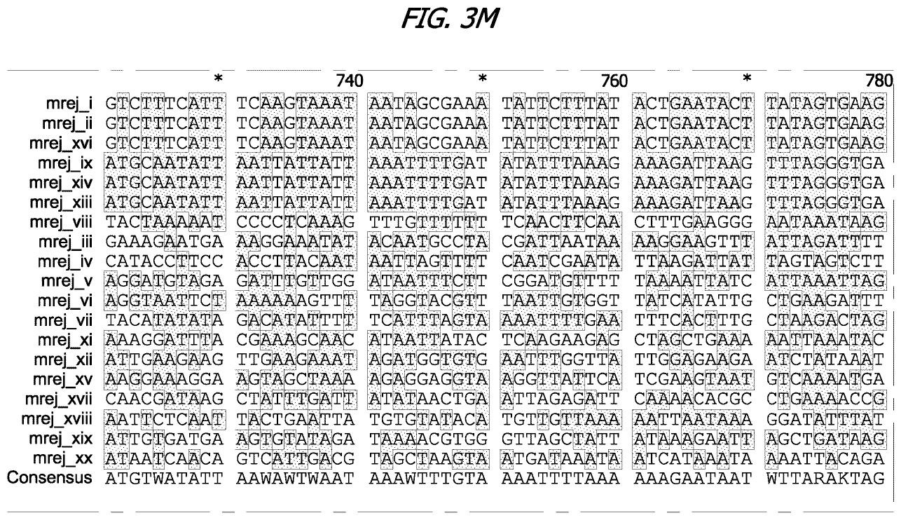

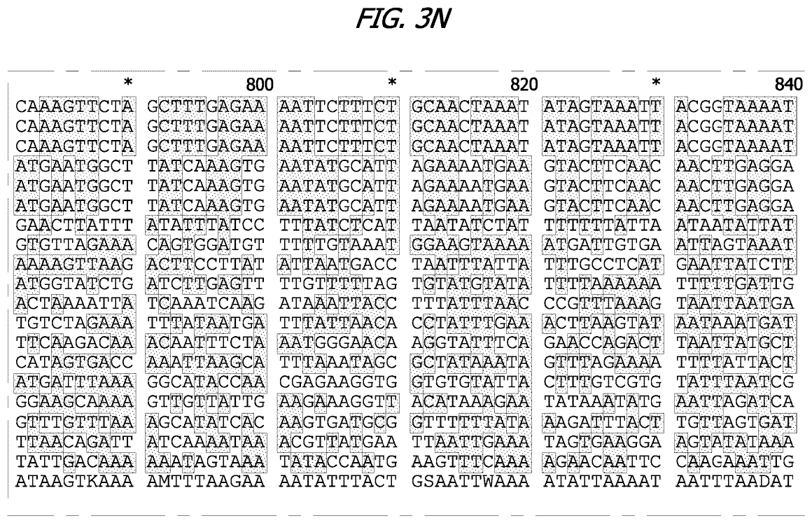

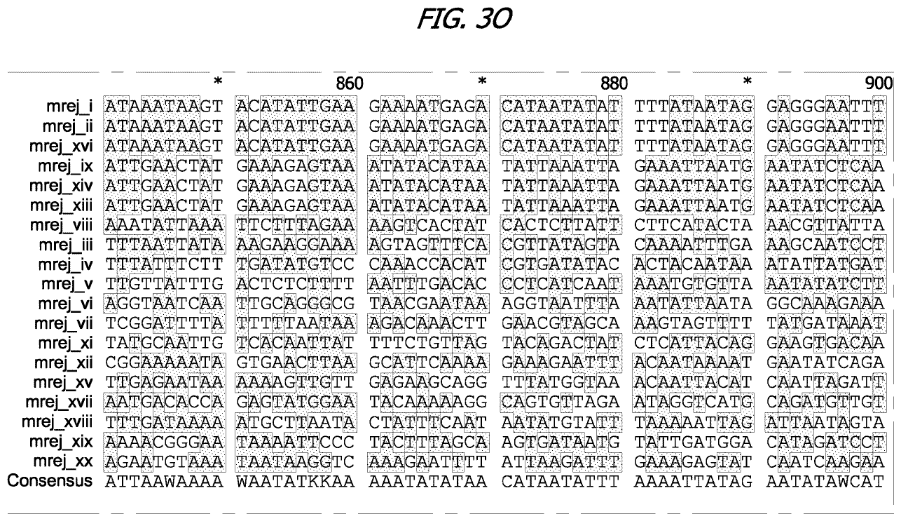

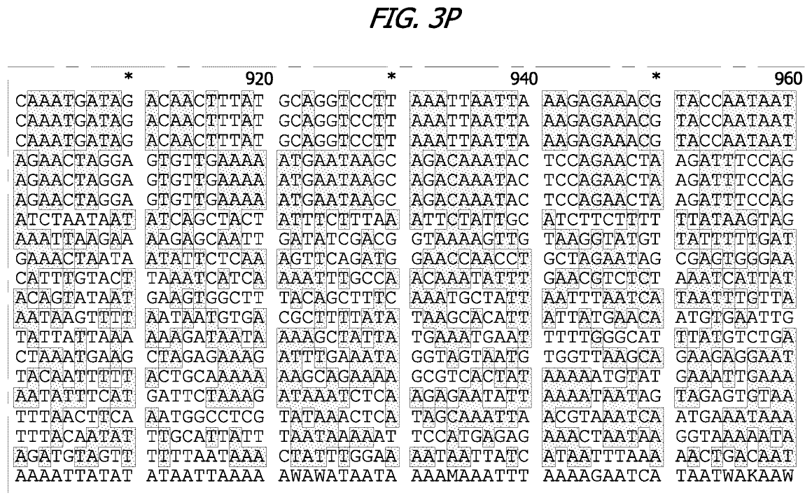

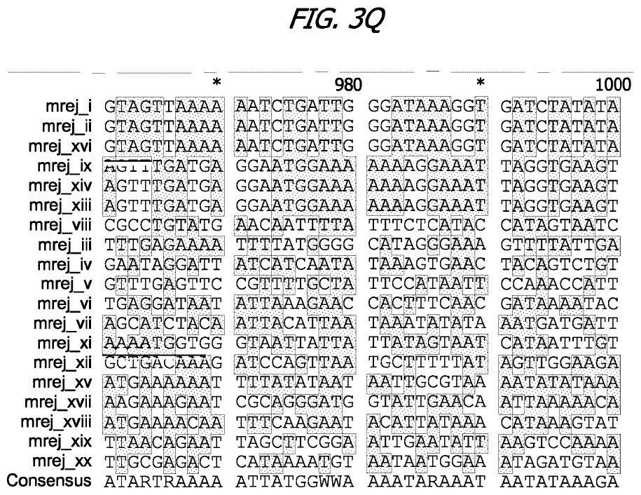

The sequences of the first 500-nucleotide portion of the SCCmec right extremity of all new MREJ types (xi to xx) were compared with each other and with those of the previously described MREJ types i to ix using GCG software programs Pileup and Gap (GCG, Wisconsin). Table 12 depicts the identities at the nucleotide level between the SCCmec right extremities of the 10 novel MREJ types (xi to xx) with those of the MREJ types previously described (i to ix) using the GCG program Gap. MREJ type x was excluded from this comparison since this MREJ sequence is deleted of the complete orfX and of the SCCmec integration site as well as ˜4 kb at the right extremity of SCCmec when compared to the right extremity of SCCmec type II. The SCCmec right extremity of MREJ types ix, xiii, and xiv differed by only one and two 102-bp insertions present in MREJ types ix and xiv, respectively. However, the rest of these three sequences showed nearly 100% identity (FIGS. 3A-3Q). Although the SCCmec portion of MREJ type xvi is nearly 100% identical with that of MREJ type ii, the deletion of nucleotides 165 to 434 of orfX in MREJ type xvi has never been described previously. The SCCmec right extremities of all other new MREJ types showed identities ranging from 38.2 to 59.5% with each other or with MREJ types i to ix. The substantial variation between the novel MREJ sequences and the previously described sequences, from which the prior detection assays were based, explains why the right extremities of the novel MREJ types xi to xx disclosed in the present invention could not have been predicted nor detected with MREJ primers previously described (U.S. Pat. No. 6,156,507; International Patent Application PCT/CA02/00824; Ito et al., 2001, Antimicrob. Agents Chemother. 45:1323-1336; Huletsky et al., 2004, J Clin. Microbiol. 42:1875-1884; Ma et al, 2002, Antimicrob. Agents Chemother. 46:1147-1152; Ito et al, Antimicrob Agents Chemother. 2004. 48:2637-2651; Oliveira et al, 2001, Microb. Drug Resist. 7:349-360).

### Example 5: Selection of Amplification Primers from SCCmec/orfX Sequences of MRSA with MREJ Types xi to xx

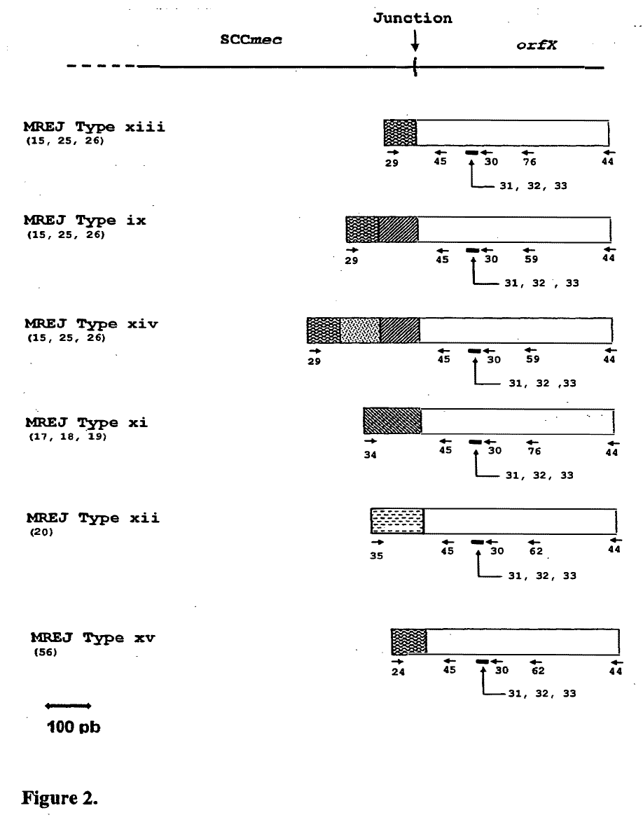

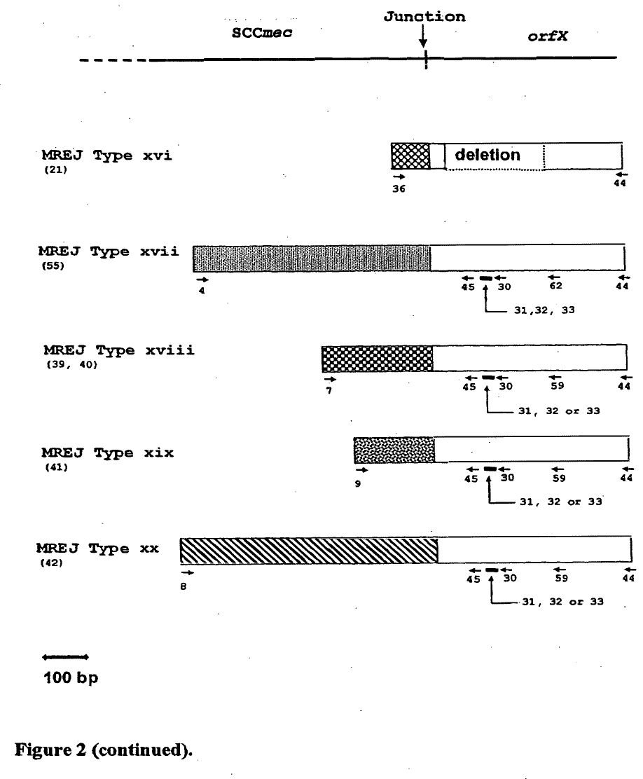

Upon analysis of the 10 new MREJ types xi to xx sequence data described above, primers specific to each new MREJ type sequence were designed (FIG. 2, Tables 9 and 11). Primers specific to MREJ type xi (SEQ ID NO.: 34), MREJ type xii (SEQ ID NO.: 35), MREJ types xiii and xiv (SEQ ID NO.: 29) (also detect MREJ type ix but each of MREJ types ix, xiii, and xiv has a different amplicon length), MREJ type xv (SEQ ID NO.: 24), MREJ type xvii (SEQ ID NO.: 4), MREJ type xviii (SEQ ID NO.: 7), MREJ type xix (SEQ ID NO.: 9), MREJ type xx (SEQ ID NO.: 8), were each used in combination with a primer specific to the S. aureus orfX (SEQ ID NO.: 30) and tested against their specific MREJ target. For the detection of MREJ type xvi, a primer targeting MREJ types i, ii, and xvi (Table 10) was used in combination with a primer targeting the S. aureus orfX (SEQ ID NO.: 44). MREJ types i, ii, and xvi can be distinguished from each other by their different amplicon length.

Oligonucleotides primers found to amplify specifically DNA from the target MRSA MREJ types were subsequently tested for their ubiquity by PCR amplification (i.e. ubiquitous primers amplified efficiently most or all isolates of MRSA of the target MREJ type). The specificity and ubiquity of the PCR assays were tested either directly with bacterial cultures or with purified bacterial genomic DNA. The specificity of the primers targeting MREJ types xi to xx was also verified by testing DNA from MRSA strains harboring all other MREJ types.

1 μl of a treated standardized bacterial suspension or of a genomic DNA preparation purified from bacteria were amplified in a 20 μl PCR reaction mixture. Each PCR reaction contained 50 mM KCl, 10 mM Tris-HCl (pH 9.0), 0.1% Triton X-100, 2.5 mM MgCl₂, 0.4 □M of each of MREJ type xi primer (SEQ ID NO.: 34), MREJ type xii primer (SEQ ID NO.: 35), MREJ types xiii and xiv primer (SEQ ID NO.: 29), MREJ type xv primer (SEQ ID NO.: 24), MREJ type xvi (SEQ ID NO.: 36), MREJ type xvii primer (SEQ ID NO.: 4), MREJ type xviii primer (SEQ ID NO.: 7), MREJ type xix primer (SEQ ID NO.: 9), or MREJ type xx primer (SEQ ID NO.: 8) which were each used in combination with 0.4 M of a S. aureus-specific primer (SEQ ID NO.: 30 or SEQ ID NO.: 44 for MREJ type xvi), 200 □M of each of the four dNTPs (Pharmacia Biotech, Piscataway, NJ), 3.3 □g/□l of BSA (SIGMA, St. Louis, MO), and 0.5 U Taq polymerase (Promega, Madison, WI) coupled with TaqStart™ Antibody (BD Biosciences, San Jose, CA).

PCR reactions were then subjected to thermal cycling: 3 min at 94° C. followed by 40 cycles of 60 seconds at 95° C. for the denaturation step, 60 seconds at 55° C. for the annealing step, and 60 seconds at 72° C. for the extension step, then followed by a terminal extension of 7 minutes at 72° C. using a standard thermocycler (PTC-200 from MJ Research Inc., Watertown, MA). Detection of the PCR products was made by electrophoresis in agarose gels (1.2%) containing 0.25 μg/ml of ethidium bromide.

Each of the MRSA strains harbouring a specific MREJ target was specifically detected with their specific MREJ primers and there was no cross-detection with non targeted MREJ types.

This invention has been described herein above, and it is readily apparent that modifications can be made thereto without departing from the spirit of this invention. These modifications are under the scope of this invention, as defined in the appended claims.

TABLE 1
Reference Staphylococcus aureus strains used in the present invention^{a}

| Strain number | Strain number | Strain number |
|---|---|---|
| Public collections (Type designation) | | |
| ATCC 6538^{b} | BM10827 (C) | 54511 (Turku I E6) |
| ATCC 13301^{b} | 3717 (EMRSA-GR1b) | 54518 (Turku II E7) |
| ATCC 23235^{b} | 97S97 (Belgian epidemic clone 1a) | 61974 (Helsinki I E1) |
| ATCC 25923^{b} | 359/96 (Berlin epidemic EMRSA IVc) | 62176 (Kotka E10) |
| ATCC 27660^{b} | 792/96 (Berlin epidemic EMRSA IVd) | 62305 (mecA- Tampere I E12) |
| ATCC 29737^{b} | 844/96 (Berlin epidemic EMRSA IVb) | 62396 (Helsinki II E2) |
| ATCC 29213^{b} | 1966/97 (Hannover area EMRSA IIIc) | 75541 (Tampere II E13) |
| ATCC 29247^{b} | 2594-2/97 (S. German EMRSA IIb) | 75916 (Helsinki V E5) |
| ATCC 33591 | 131/98 (S. German EMRSA II d2) | 76167 (Kemi E17) |
| ATCC 33592 | 406/98 (N. German EMRSA I c1) | 98442 (Helsinki VI E19) |
| ATCC 33593 | 408/98 (N. German EMRSA I c2) | 98514 (Helsinki VII E20) |
| ATCC 43300 | 872/98 (Hannover area EMRSA IIIb) | 98541 (Lohja E24) |
| ATCC BAA-38 (Archaic)^{c} | 1155-1/98 (S. German EMRSA II c) | M307 (EMRSA-3) |
| ATCC BAA-39 (Hungarian )^{c} | 1163/98 (S. German EMRSA II d1) | 90/10685 (EMRSA-15) |
| ATCC BAA-40 (Portuguese)^{c} | 1869/98 (N. German EMRSA I d) | 98/14719 (EMRSA-15/b4) |
| ATCC BAA-41 (New York )^{c} | HS 2 (I) | 96/32010 (EMRSA-16) |
| ATCC BAA-42 (Pediatric)^{c} | AO 17934/97 (II) | 99/579 (EMRSA-16/a3) |
| ATCC BAA-43 (Brazilian )^{c} | 98/10618 (EMRSA-15/b2) | 5 (E1) |
| ATCC BAA-44 (Iberian)^{c} | 98/26821 (EMRSA-15/b3) | 3680 (EMRSA-GR1) |
| CCUG 41787 (Sa 501 V)^{e} | 98/24344 (EMRSA-15/b7) | 3713 (EMRSA-GR1a) |
| CCUG 38266 (II)^{e} | 99/1139 (EMRSA-16/a2) | 98S46 (Belgian epidemic clone 3b) |
| NCTC 8325b | 99/159 (EMRSA-16/a14) | 97S96 (Belgian epidemic clone 1a) |
| NCTC 11939 (EMRSA-1)^{e} | 6 (D) | 97S98 (Belgian epidemic clone 1b) |
| | 13 (A') | 97S99 (Belgian epidemic clone 2a) |
| Canadian epidemic MRSA (Type designation)^{d} | 14 (A') | 97S100 (Belgian epidemic clone 2b) |
| CMRSA-1 | 18 (A) | 97S101 (Belgian epidemic clone 3a) |
| CMRSA-2 | 25 (F') | 134/93 (N. German EMRSA I) |
| CMRSA-3 | 30 (G) | 1000/93 (Hannover area EMRSA III) |
| CMRSA-4 | 33 (F) | 1450/94 (N. German EMRSA Ia) |
| CMRSA-5 | 54 (B) | 825/96 (Berlin epidemic EMRSA IV) |
| CMRSA-6 | 60 (A″) | 842/96 (Berlin epidemic EMRSA IVa) |
| | 80 (E) | 2594-1/97 (S. German EMRSA II a) |
| HARMONY collection of European epidemic | 98 (C) | 1155-2/98 (S. German EMRSA II ) |
| MRSA (Type designation)^{e} | | |
| 96158 (B) | 162 (A) | 1442/98 (Hannover area EMRSA IIIa) |
| 97117 (A) | 920 (B) | N8-890/99 (Sa 543 VI) |
| 97118 (A) | 95035 (A) | N8-3756/90 (Sa544 I) |
| 97120 (B) | 97121 (B) | 9805-01937 (V) |
| 97151 (B) | BM10828 (C) | AK 541 (IV) |
| 97392 (B) | BM10882 (C) | ON 408/99 (VII) |
| 97393 (A) | 37481 (Seinajoki E 14) | AO 9973/97 (III) |

^{a}All S. aureus strains are resistant to methicillin except where otherwise indicated.
^{b}These S. aureus strains are sensitive to oxacillin (MSSA).
^{c}Informations on these strains and type designation based on pulse-field gel electrophoresis are from (6).
dInformation on these strains and type designation based on pulse-field gel electrophoresis are from (47).
^{e}Information on these strains and type designation based on pulse-field gel electrophoresis are available at the world wide web site located at phls.co.uk/inter/harmony/menu.htm.

TABLE 2
Evaluation of the MRSA-specific primers targeting MREJ types i to x using DNA
from a variety of methicillin-sensitive and methicillin-resistant Staphylococcus
aureus strains.

| Staphylococcus aureus strainsª | PCR results | |
|---|---|---|
| (number) | Positive (%) | Negative (%) |
| MRSA (1657) | 1640 (99) | 17 (1) |
| MSSA (569) | 26 (4.6) | 543 (95.4) |

^{a}MRSA, methicillin-resistant Staphylococcus aureus; MSSA, methicillin-sensitive Staphylococcus aureus. Reference S. aureus strains used are listed in Table 1. The origin of the S. aureus clinical isolates is described in the text.

TABLE 3
Origin of 17 MRSA strains not amplifiable using primers targeting MREJ
types i to x.

| Staphylococcus aureus | | |
|---|---|---|
| strain designation: | | |
| Original | CCRIª | Origin |
| 6-9637 | CCRI-12157 | Tempe, USA |
| 15-3967 | CCRI-12198 | New York, USA |
| 15-3972 | CCRI-12199 | New York, USA |
| 91 2290 | CCRI-12719 | Australia |
| SS1757 | CCRI-11976 | Houston, USA |
| 255 D | CCRI-12382 | Brazil |
| 106 I | CCRI-12383 | Brazil |
| 232 D | CCRI-12376 | Brazil |
| 6881 | CCRI-12593 | Spain |
| 5109 | CCRI-11999 | Wilmington, USA |
| BK793 | CCRI-9887 | Cairo, Egypt |
| 21 1 8424 | CCRI-12845 | Japan |
| SE46-1 | CCRI-9772 | Toronto, Canada |
| 1059 | CCRI-12524 | Italy |
| 1016 | CCRI-12535 | Italy |
| 816867 | CCRI-12905 | Rennes, France |
| 20 1 6060 | CCRI-12810 | Taiwan, China |

^{a}CCRI stands for ″Collection of the Centre de Recherche en Infectiologie″.

TABLE 8
Novel Staphylococcus aureus MREJa nucleotide sequences

| | S. aureus strain designation | | |
|---|---|---|---|
| SEQ ID | Original | CCRI^{c} | Sequence^{a,b} |
| 15 | SS1757 | CCRI-11976 | MREJ type xiii |
| 16 | 5109 | CCRI-11999 | MREJ type xiv |
| 17 | 6-9637 | CCRI-12157 | MREJ type xi |
| 18 | 15-3967 | CCRI-12198 | MREJ type xi |
| 19 | 15-3962 | CCRI-12199 | MREJ type xi |
| 20 | 91 2290 | CCRI-12719 | MREJ type xii |
| 21 | 21 1 8424 | CCRI-12845 | MREJ type xvi |
| 25 | 255 D | CCRI-12382 | MREJ type xiii |
| 26 | 106 I | CCRI-12383 | MREJ type xiii |
| 39 | 1059 | CCRI-12524 | MREJ type xviii |
| 40 | 1016 | CCRI-12535 | MREJ type xviii |
| 41 | 20 1 6060 | CCRI-12810 | MREJ type xix |
| 42 | 816867 | CCRI-12095 | MREJ type xx |
| 55 | SE46-1 | CCRI-9772 | MREJ type xvii |
| 56 | BK793 | CCRI-9887 | MREJ type xv |

^{a}MREJ refers to mec right extremity junction and includes sequences from the SCCmec right extremity and chromosomal DNA to the right of the SCCmec integration site.
^{b}Sequence refers to the target gene
^{c}CCRI stands for ″Collection for the Centre de Recherche en Infectiologie″

TABLE 9
Novel PCR amplification primers developed to detect MREJ types xi - xx

| Originating target DNA | Originating target | | Oligo SEQ ID |
|---|---|---|---|
| MREJ type | DNA SEQ ID NO | Oligo Positionª | NO |
| MREJ type xvii | 55 | 954^{b} | 4 |
| MREJ type xviii | 40 | 1080 | 7 |
| MREJ type xx | 42 | 987^{b} | 8 |
| MREJ type xix | 41 | 581^{b} | 9 |
| MREJ type XV | 38 | 624 | 23 |
| MREJ type xV | 56 | 566^{b} | 24 |
| MREJ type ix, xiii, xiv | 15 | 756^{b} | 28 |
| MREJ type xi | 17 | 615 | 34 |
| MREJ type xii | 20 | 612^{b} | 35 |
| MREJ type xV | 56 | 457 | 48 |
| MREJ type xV | 56 | 564^{b} | 49 |
| MREJ type xi | 17 | 956^{b} | 51 |
| MREJ type xii | 20 | 1053^{b} | 52 |
| MREJ type xvii | 55 | 415 | 57 |
| MREJ type xvii | 55 | 558 | 58 |

^{a}Position refers to nucleotide position of 5' end of primer
^{b}Primer is reverse-complement of target sequence

TABLE 10
Other amplification and/or sequencing primers and probes found in the
sequence listing

| SEQ ID | | | Originating DNA | |
|---|---|---|---|---|
| NO | Source | Target | Position^{a} | SEQ ID NO |
| 27 | Oliveira and de Lencastre. 2002, | SCCmec | — | — |
| | Antimicrob. Agents Chemother. | | | |
| | 46: 2155-2161 | | | |
| 29 | International Patent Application | MREJ types ix, xiii, and | 652^{c} | 29 |
| | PCT/CA02/00824.^{b} | xiv | | |
| 30 | International Patent Application | orfX | 325 | 18 |
| | PCT/CA02/00824.^{b} | | | |
| 31 | International Patent Application | orfX | 346^{c} | 18 |
| | PCT/CA02/00824.^{b} | | | |
| 32 | International Patent Application | orfX | 346^{c} | 20 |
| | PCT/CA02/00824.^{b} | | | |
| 33 | International Patent Application | orfX | — | — |
| | PCT/CA02/00824.^{b} | | | |
| 36 | International Patent Application | MREJ types i, ii, and xvi | 574^{c} | 21 |
| | PCT/CA02/00824.^{b} | | | |
| 43 | International Patent Application | orfX | 367^{c} | 18 |
| | PCT/CA02/00824.^{b} | | | |
| 44 | International Patent Application | orfX | 98 | 38 |
| | PCT/CA02/00824.^{b} | | | |
| 45 | International Patent Application | orfX | 401 | 18 |
| | PCT/CA02/00824.^{b} | | | |
| 50 | International Patent Application | mecA | 6945^{c} | 22 |
| | PCT/CA02/00824.^{b} | | | |
| 53 | Oliveira and de Lencastre, 2002, | SCCmec | — | — |
| | Antimicrob. Agents Chemother. | | | |
| | 46: 2155-2161 | | | |
| 54 | International Patent Application | MREJ types i and ii | — | — |
| | PCT/CA02/00824.^{b} | | | |
| 60 | WO96/08582^{d} | putative | membrane | |
| | | protein | | |
| 61 | WO96/08582^{d} | putative | membrane | |
| | | protein | | |
| 62 | This patent | orfX | 193 | 20 |
| 63 | International Patent Application | mecA | 6798 | 22 |
| | PCT/CA02/00824.^{b} | | | |
| 65 | International Patent Application | MREJ type vi | 642^{c} | 191^{b} |
| | PCT/CA02/00824.^{b} | | | |
| 66 | International Patent Application | MREJ types ii, viii, ix, | 514 | 167^{b} |
| | PCT/CA02/00824.^{b} | xiii, xiv | | |
| 73 | This patent | MREJ type x | 1913^{c} | 69 |
| 74 | International Patent Application | MREJ type vii | 503 | 189^{b} |
| | PCT/CA02/00824.^{b} | | | |
| 75 | International Patent Application | MREJ type viii | 601 | 167^{b} |
| | PCT/CA02/00824.^{b} | | | |
| 76 | This patent | orfX | 193 | 17 |

^{a}Position refers to nucleotide position of the 5′ end of primer (on the target sequence).
^{b}SEQ ID NOs from International Patent Application PCT/CA02/00824.
^{c}Primer is reverse-complement of target sequence.
^{d}SEQ ID NOs from WO96/08582.

TABLE 11
Length of amplicons obtained with primer pairs for MREJ types xi - xx

| Oligo Pair | | |
|---|---|---|
| (SEQ ID NO) | Target DNA | Amplicon lengthª |
| 24/30 | MREJ type xv | 265 |
| 24/44 | MREJ type xv | 603 |
| 24/45 | MREJ type xv | 189 |
| 24/62 | MREJ type xv | 397 |
| 28/30 | MREJ type xiii, xiv | 464 (type xiii); 668 (type xiv) |
| 28/44 | MREJ type xiii, xiv | 802^{b} (type xiii); 1006^{b} (type xiv) |
| 28/45 | MREJ type xiii, xiv | 388 (type xiii); 592 (type xiv) |
| 28/76 | MREJ type xiii | 596 (type xiii) |
| 29/30 | MREJ type xiii, xiv | 267 (type xiii); 471 (type xiv) |
| 29/44 | MREJ type xiii, xiv | 605^{b} (type xiii); 809^{b} (type xiv) |
| 29/45 | MREJ type xiii, xiv | 191 (type xiii); 395 (type xiv) |
| 29/59 | MREJ type xiv | 605 |
| 29/76 | MREJ type xiii | 399 |
| 34/30 | MREJ type xi | 328 |
| 34/44 | MREJ type xi | 661^{b} |
| 34/45 | MREJ type xi | 247 |
| 34/76 | MREJ type xi | 455 |
| 35/30 | MREJ type xii | 311 |
| 35/44 | MREJ type xii | 649^{b} |
| 35/45 | MREJ type xii | 235 |
| 35/62 | MREJ type xii | 443 |
| 36/44 | MREJ type xvi | 348^{b} |
| 4/30 | MREJ type xvii | 690 |
| 4/44 | MREJ type xvii | 968^{b} |
| 4/45 | MREJ type xvii | 614 |
| 4/62 | MREJ type xvii | 822 |
| 7/30 | MREJ type xviii | 780^{b} |
| 7/44 | MREJ type xviii | 1119^{b} |
| 7/45 | MREJ type xviii | 704 |
| 7/59 | MREJ type xviii | 912b |
| 8/30 | MREJ type xx | 1076^{b} |
| 8/44 | MREJ type xx | 1415^{b} |
| 8/45 | MREJ type xx | 1000 |
| 8/59 | MREJ type xx | 1208^{b} |
| 9/30 | MREJ type xix | 657^{b} |
| 9/44 | MREJ type xix | 996^{b} |
| 9/45 | MREJ type xix | 581 |
| 9/59 | MREJ type xix | 789^{b} |

^{a}Amplicon length is given in base pairs for MREJ types amplified by the set of primers
^{b}Amplicon length is based on analysis by agarose gel electrophoresis

TABLE 12
Percentage of sequence identity for the first 500 nucleotides of SCCmec right extremities between 19 types of MREJ^{a,b}

| | i | ii^{d} | iii | iv | v | vi^{c} | vii | viii | ix^{f} | xi | xii | xiii | xiv^{e} | xv | xvi | xvii | xviii | xix | xx |
|---|---|---|---|---|---|---|---|---|---|---|---|---|---|---|---|---|---|---|---|
| i | — | 100 | 44.4 | 39.1 | 40.4 | 42.9 | 43.2 | 41.5 | 42.4 | 41.1 | 40.2 | 42.4 | 42.4 | 42.1 | 100 | 42.1 | 44.1 | 42.5 | 40.4 |
| ii^{d} | — | — | 44.4 | 39.1 | 40.4 | 42.9 | 43.2 | 41.5 | 42.4 | 41.1 | 40.2 | 42.4 | 42.4 | 42.1 | 100 | 42.1 | 44.1 | 42.5 | 40.4 |
| iii | — | — | — | 40.1 | 45.8 | 45.0 | 44.4 | 42.3 | 49.9 | 45.7 | 44.9 | 49.9 | 49.9 | 48.8 | 44.2 | 43.1 | 46.7 | 43.5 | 45.3 |
| iv | — | — | — | — | 45.2 | 40.3 | 41.6 | 42.1 | 42.7 | 42.7 | 38.2 | 42.7 | 42.7 | 39.0 | 42.1 | 39.6 | 40.3 | 37.2 | 41.4 |
| v | — | — | — | — | — | 45.0 | 41.3 | 46.5 | 43.8 | 41.2 | 43.6 | 43.8 | 43.8 | 41.7 | 43.3 | 42.3 | 49.8 | 43.8 | 39.8 |
| vi^{c} | — | — | — | — | — | — | 45.1 | 40.8 | 43.2 | 43.8 | 42.0 | 43.2 | 43.2 | 44.2 | 42.9 | 39.4 | 43.4 | 42.2 | 45.8 |
| vii | — | — | — | — | — | — | — | 42.8 | 44.8 | 42.7 | 42.0 | 44.8 | 44.8 | 46.7 | 42.5 | 43.2 | 44.7 | 41.1 | 41.9 |
| viii | — | — | — | — | — | — | — | — | 41.1 | 41.1 | 41.9 | 41.1 | 41.1 | 37.6 | 52.2 | 40.9 | 41.2 | 42.4 | 39.1 |
| ix^{f} | — | — | — | — | — | — | — | — | — | 46.0 | 42.5 | 100 | 100 | 43.4 | 44.1 | 40.9 | 45.0 | 40.6 | 42.9 |
| xi | — | — | — | — | — | — | — | — | — | — | 48.0 | 46.0 | 46.0 | 45.9 | 43.2 | 40.5 | 47.1 | 41.8 | 43.6 |
| xii | — | — | — | — | — | — | — | — | — | — | — | 42.5 | 42.5 | 47.6 | 39.2 | 43.3 | 43.7 | 44.7 | 45.6 |
| xiii | — | — | — | — | — | — | — | — | — | — | — | — | 100 | 43.4 | 44.1 | 40.9 | 45.0 | 40.6 | 42.9 |
| xiv^{e} | — | — | — | — | — | — | — | — | — | — | — | — | — | 43.4 | 44.1 | 40.9 | 45.0 | 40.6 | 42.9 |
| xv | — | — | — | — | — | — | — | — | — | — | — | — | — | — | 43.6 | 41.5 | 47.5 | 42.5 | 43.8 |
| xvi | — | — | — | — | — | — | — | — | — | — | — | — | — | — | — | 41.9 | 45.4 | 45.1 | 45.7 |
| xvii | — | — | — | — | — | — | — | — | — | — | — | — | — | — | — | — | 40.0 | 43.3 | 42.5 |
| xviii | — | — | — | — | — | — | — | — | — | — | — | — | — | — | — | — | — | 43.4 | 45.7 |
| xix | — | — | — | — | — | — | — | — | — | — | — | — | — | — | — | — | — | — | 45.5 |
| xx | — | — | — | — | — | — | — | — | — | — | — | — | — | — | — | — | — | — | — |

^{a}“First 500 nucleotides” refers to the 500 nucleotides within the SCCmec right extremity, starting from the integration site of SCCmec in the Staphylococcus aureus chromosome as shown on FIGS. 3A-3Q.
^{b}Sequences were extracted from International patent application PCT/CA02/00824 (SEQ ID NOs.: 1, 2, 232, 46, 50, 171, 166, 167 and 168 and correspond to SEQ ID NOs: 89-97 of the present application, respectively, for types i to ix). MREJ type x was excluded from the sequence comparison because it is deleted from the completed orfX, the integration site, and part of the SCCmec right extremity. Sequences for types xi to xx were extracted from SEQ ID NOs.: 18, 20, 25, 16, 56, 21, 55, 39, 41 and 42, respectively.
^{c}Sequence from the SCCmec right extremity of MREJ type vi is limited to 371 nucleotides.
^{d}The first 102 nucleotides from the SCCmec right extremity of MREJ type ii were excluded from the sequence comparison.
^{e}The first 206 nucleotides from the SCCmec right extremity of MREJ type xiv were excluded from the sequence comparison.
^{f}The first 102 nucleotides from the SCCmec right extremity of MREJ type ix were excluded from the sequence comparison.

1-53. (canceled)

54. A method of treating a methicillin resistant Staphylococcus aureus (MRSA) in a subject comprising:
contacting or having contacted under annealing conditions a plurality of primers comprising a first primer and a second primer with a sample from the subject to be analyzed for the presence of an MREJ type xiv sequence, said MREJ type xiv sequence from a MRSA strain including a SCCmec element containing a mecA gene inserted into chromosomal DNA, thereby generating a polymorphic MREJ type xiv sequence that comprises sequences from both the SCCmec element right extremity and chromosomal DNA adjoining said right extremity, wherein said first and second primers are at least 16 nucleotides in length, wherein said first primer anneals to said SCCmec element right extremity of a MREJ type xiv sequence consisting of SEQ ID NO: 16, and said second primer anneals to a chromosomal sequence of S. aureus, wherein said first primer and said second primer together generate a first amplicon of MREJ type xiv specific sequence that spans the mec right extremity junction of the MREJ type xiv sequence under amplification conditions only if said MREJ type xiv sequence is present in the sample from the subject;
generating and detecting, or having generated and detected, the presence of said first amplicon of MREJ type xiv specific sequence as indicative of the presence of MRSA in the sample from the subject; and
treating the MRSA in the subject by administering an antibiotic to the subject.

55. The method of claim 54, wherein said detecting said first amplicon comprises hybridization with at least one probe.

56. The method of claim 55, wherein said contacting of the plurality of primers and the hybridization with at least one probe occurs in the same physical enclosure.

57. The method of claim 55, wherein the plurality of primers and the at least one probe are each at least 16 nucleotides in length.

58. The method of claim 55, wherein the plurality of primers further comprises primers for the detection of additional MREJ type nucleic acid sequences that anneal to the SCCmec element right extremity of a MREJ sequence of each of SEQ ID NOs: 15, 17, 18, 19, 20, 21, 25, 26, 39, 40, 41, 55 and 56.

59. The method of claim 55, wherein the plurality of primers and the at least one probe comprise at least one primer or probe selected from the group consisting of SEQ ID NOs: 44, 45, 76, 51, 30, 31, 32, and 33 for the detection of MREJ type xi.

60. The method of claim 55, wherein the plurality of primers and the at least one probe comprise at least one primer or probe selected from the group consisting of SEQ ID NOs: 35, 44, 45, 62, 52, 30, 31, 32, and 33, for the detection of MREJ type xii.

61. The method of claim 55, wherein the plurality of primers and the at least one probe comprise at least one primer or probe selected from the group consisting of SEQ ID NOs: 28, 44,45,59, 29, 30, 31, 32, and 33 for the detection of MREJ type xiii.

62. The method of claim 55, wherein the plurality of primers and the at least one probe comprise at least one primer or probe selected from the group consisting of SEQ ID NOs: 44, 45, 59, 29, 30, 31, 32, and 33 for the detection of MREJ type xiv.

63. The method of claim 55, wherein the plurality of primers and the at least one probe comprise at least one primer or probe selected from the group consisting of SEQ ID NOs: 44, 45, 62, 24, 30, 31, 32, and 33 for the detection of MREJ type xv.

64. The method of claim 55, wherein the plurality of primers and the at least one probe comprise at least one primer or probe selected from the group consisting of SEQ ID NOs: 36 and 44 for the detection of MREJ type xvi.

65. The method of claim 55, wherein the plurality of primers and the at least one probe comprise at least one primer or probe selected from the group consisting of SEQ ID NOs: 44, 45, 57, 58, 62, 4, 30, 31, 32, and 33 for the detection of MREJ type xvii.

66. The method of claim 55, wherein the plurality of primers and the at least one probe comprise at least one primer or probe selected from the group consisting of SEQ ID NOs: 44, 45, 59, 7, 30, 31, 32, and 33 for the detection of MREJ type xviii.

67. The method of claim 55, wherein the plurality of primers and the at least one probe comprise at least one primer or probe selected from the group consisting of SEQ ID NOs: 44, 45, 59, 9, 30, 31, 32, and 33 for the detection of MREJ type xix.

68. The method of claim 55, wherein the plurality of primers and the at least one probe comprise at least one primer or probe selected from the group consisting of SEQ ID NOs: 44, 45, 59, 8, 30, 31, 32, and 33 for the detection of MREJ type xx.

69. The method of claim 58, wherein the plurality of primers and the at least one probe comprise a plurality of primers or probes selected from the group consisting of:
SEQ ID NOs: 51, 30, 31, 32, 33, 52, 29, 24, 36, 44, 4, 7, and 9.

70. The method of claim 69, wherein the plurality of primers or probes further comprise at least one primer and/or probe selected from the group consisting of: SEQ ID NOs: 34, 35, 44, 45, 52, 59, 62, and 76.

71. The method of claim 54, wherein the plurality of primers comprise a primer pair, wherein the primer pair is selected from the group consisting of:
SEQ ID NOs: 34 and 45; SEQ ID NOs: 34 and 30; SEQ ID NOs: 34 and 76; and
SEQ ID NOs: 34 and 44 for the detection of MREJ type xi.

72. The method of claim 54, wherein the plurality of primers comprise a primer pair, wherein a primer pair is selected from the group consisting of:
SEQ ID NOs: 35 and 45; SEQ ID NOs: 35 and 30; SEQ ID NOs: 35 and 62; and
SEQ ID NOs: 35 and 44, for the detection of MREJ type xii.

73. The method of claim 54, wherein the plurality of primers comprise a primer pair, wherein the primer pair is selected from the group consisting of:
SEQ ID NOs: 29 and 45; SEQ ID NOs: 29 and 30; SEQ ID NOs: 29 and 76; and
SEQ ID NOs: 29 and 44, for the detection of MREJ type xiii.

74. The method of claim 54, wherein the plurality of primers comprise a primer pair, wherein the primer pair is selected from the group consisting of:
SEQ ID NOs: 29 and 45; SEQ ID NOs: 29 and 30; SEQ ID NOs: 29 and 59; and
SEQ ID NOs: 29 and 44, for the detection of MREJ type xiv.

75. The method of claim 54, wherein the plurality of primers comprise a primer pair, wherein the primer pair is selected from the group consisting of:
SEQ ID NOs: 24 and 45; SEQ ID NOs: 24 and 30; SEQ ID NOs: 24 and 62; and
SEQ ID NOs: 24 and 44, for the detection of MREJ type xv.

76. The method of claim 54, wherein the plurality of primers comprise a primer pair, wherein the primer pair is SEQ ID NOs: 36 and 44, for detection of MREJ type xvi.

77. The method of claim 54, wherein the plurality of primers comprise a primer pair, wherein the primer pair is selected from the group consisting of:
SEQ ID NOs: 4 and 45; SEQ ID NOs: 4 and 30; SEQ ID NOs: 4 and 62; and SEQ ID NOs: 4 and 44, for the detection of MREJ type xvii.

78. The method of claim 54, wherein the plurality of primers comprise a primer pair, wherein the primer pair is selected from the group consisting of:
SEQ ID NOs: 7 and 45; SEQ ID NOs: 7 and 30; SEQ ID NOs: 7 and 59; and SEQ ID NOs: 7 and 44, for the detection of MREJ type xviii.

79. The method of claim 54, wherein the plurality of primers comprise a primer pair, wherein the primer pair is selected from the group consisting of:
SEQ ID NOs: 9 and 45; SEQ ID NOs: 9 and 30; SEQ ID NOs: 9 and 59; and SEQ ID NOs: 9 and 44, for the detection of MREJ type xix.

80. The method of claim 54, wherein the plurality of primers comprise a primer pair, wherein the primer pair is selected from the group consisting of:
SEQ ID NOs: 8 and 45, SEQ ID NOs: 8 and 30; SEQ ID NOs: 8 and 59; and SEQ ID NOs: 8 and 44, for the detection of xx.

81. The method of claim 54, wherein the plurality of primers further comprise a primer pair for the detection of an additional MREJ type nucleic acid sequences, wherein the primer pair is selected from the group consisting of:
SEQ ID NOs: 34 and 45; SEQ ID NOs: 34 and 30; SEQ ID NOs: 34 and 76; SEQ ID NOs: 34 and 44; SEQ ID NOs: 35 and 45; SEQ ID NOs: 35 and 30; SEQ ID NOs: 35 and 62; SEQ ID NOs: 35 and 44; SEQ ID NOs: 29 and 45; SEQ ID NOs: 29 and 30; SEQ ID NOs: 29 and 76; SEQ ID NOs: 29 and 44; SEQ ID NOs: 29 and 45; SEQ ID NOs: 29 and 30; SEQ ID NOs: 29 and 59; SEQ ID NOs: 29 and 44; SEQ ID NOs: 36 and 44; SEQ ID NOs: 4 and 45; SEQ ID NOs: 4 and 30; SEQ ID NOs: 4 and 62, SEQ ID NOs: 4 and 44; SEQ ID NOs: 7 and 45; SEQ ID NOs: 7 and 30; SEQ ID NOs: 7 and 59; SEQ ID NOs:
7 and 44; SEQ ID NOs: 9 and 45; SEQ ID NOs: 9 and 30; SEQ ID NOs: 9 and 59; SEQ ID NOs: 9 and 44; SEQ ID NOs: 8 and 45; SEQ ID NOs: 8 and 30; SEQ ID NOs: 8 and 59; and SEQ ID NOs: 8 and 44.

82. The method of claim 81, further comprising probes comprising a nucleic acid sequence selected from the group consisting of: SEQ ID NOs: 33, 31, and 32.

83. The method of claim 82, wherein said probes and primers are used together.

84. The method of claim 83, wherein said probes and/or primers are used together in the same physical enclosure.

85. The method of claim 54, wherein the detecting comprises detecting an annealed probe and/or primer as an indication of the presence of said MREJ type xiv sequence in the sample.

86. The method of claim 54, wherein the detecting the presence of said first amplicon comprises a method selected from the group consisting of: agarose gel electrophoresis, fluorescence resonance energy transfer, chemiluminescence, potentiometry, mass spectrometry, plasmon resonance, polarimetry, colorimetry, flow cytometry, scanometry, and DNA sequencing, or any combination thereof.

87. The method of claim 54, further comprising contacting the sample with at least one primer pair selected from the group consisting of: SEQ ID NOs: 30 and 36; SEQ ID NOS: 30 and 70; SEQ ID NOs: 30 and 71; SEQ ID NOs: 30 and 72; SEQ ID NOs: 30 and 65; SEQ ID NOs: 30 and 74; SEQ ID NOs: 30 and 29; and SEQ ID NOs: 73 and 77 under said amplifying conditions.

88. The method of claim 87, wherein said contacting further comprises providing at least one probe, wherein the probe comprises a nucleic acid sequence selected from the group consisting of: SEQ ID NO:31, SEQ ID NO:32, and SEQ ID NO:33.

89. The method of claim 54, wherein said detecting comprises sequencing said first amplicon.

90. The method of claim 54, wherein said detecting comprises determining the size of said first amplicon by gel electrophoresis.

91. The method of claim 54, further comprising detecting the presence of at least one further MREJ type nucleic acid sequence in said sample, said at least one further MREJ type nucleic acid sequence being an MREJ type xi, xii, xiii, xv, xvi, xvii, xviii, xix, or xx MRSA strain, comprising:
contacting at least a third primer with the sample, said MREJ type xi, xii, xv, xvi, xvii, xviii, xix, or xx type sequence from a MRSA strain including a Staphylococcal cassette chromosome mec (SCCmec) element containing a mecA gene inserted into chromosomal DNA, thereby generating a polymorphic MREJ type xi, xii, xiii, xv, xvi, xvii, xviii, xix, or xx sequence that comprises sequences from both the SCCmec right extremity and chromosomal DNA adjoining said right extremity, wherein said third primer and a primer that anneals to a chromosomal sequence of S. aureus together generate a second amplicon that spans the mec right extremity junction of the MREJ type xi, xii, xiii, xv, xvi, xvii, xviii, xix, or xx sequence under amplification conditions only if said MREJ type xi, xii, xiii, xv, xvi, xvii, xviii, xix, or xx sequence, respectively, is present in the sample and wherein said contacting takes place under annealing conditions; and
generating and detecting the presence of said second amplicon as indicative of the presence of said MREJ type xi, xii, xiii, xv, xvi, xvii, xviii, xix, or xx sequence in the sample.

92. The method of claim 54, wherein said first and second primers are at least 18 nucleotides in length.


SEQUENCE LISTING (97 sequences)

SEQ ID NO: 1 - DNA, 21 bp, synthetic construct
  misc_feature 1..21: note = Synthetically prepared nucleic acid sequence
  source 1..21: mol_type = other DNA
gcaggaacaaacagatgaaga

SEQ ID NO: 2 - DNA, 19 bp, synthetic construct
  misc_feature 1..19: note = Synthetically prepared nucleic acid sequence
  source 1..19: mol_type = other DNA
tcggctctaccctcaacaa

SEQ ID NO: 3 - DNA, 19 bp, synthetic construct
  misc_feature 1..19: note = Synthetically prepared nucleic acid sequence
  source 1..19: mol_type = other DNA
tcgcagggatggtattgaa

SEQ ID NO: 4 - DNA, 19 bp, synthetic construct
  misc_feature 1..19: note = Synthetically prepared nucleic acid sequence
  source 1..19: mol_type = other DNA
caccctgcaagatatgttt

SEQ ID NO: 5 - DNA, 25 bp, synthetic construct
  misc_feature 1..25: note = Synthetically prepared nucleic acid sequence
  source 1..25: mol_type = other DNA
ttcgttgaaagaagagaaaattaaa

SEQ ID NO: 6 - DNA, 27 bp, synthetic construct
  misc_feature 1..27: note = Synthetically prepared nucleic acid sequence
  source 1..27: mol_type = other DNA
gcttttttttctttattatcaagtatc

SEQ ID NO: 7 - DNA, 25 bp, synthetic construct
  misc_feature 1..25: note = Synthetically prepared nucleic acid sequence
  source 1..25: mol_type = other DNA
aatggaatttgttaatttcataaat

SEQ ID NO: 8 - DNA, 25 bp, synthetic construct
  misc_feature 1..25: note = Synthetically prepared nucleic acid sequence
  source 1..25: mol_type = other DNA
ttccgaagtcataatcaatcaaatt

SEQ ID NO: 9 - DNA, 23 bp, synthetic construct
  misc_feature 1..23: note = Synthetically prepared nucleic acid sequence
  source 1..23: mol_type = other DNA
ttccgaagctaattctgttaata

SEQ ID NO: 10 - DNA, 23 bp, synthetic construct
  misc_feature 1..23: note = Synthetically prepared nucleic acid sequence
  source 1..23: mol_type = other DNA
aggcgttaaaaatcctgatactg

SEQ ID NO: 11 - DNA, 22 bp, synthetic construct
  misc_feature 1..22: note = Synthetically prepared nucleic acid sequence
  source 1..22: mol_type = other DNA
aagccaattcaatttgtaatgc

SEQ ID NO: 12 - DNA, 22 bp, synthetic construct
  misc_feature 1..22: note = Synthetically prepared nucleic acid sequence
  source 1..22: mol_type = other DNA
aacccctcctctgtaattagtg

SEQ ID NO: 13 - DNA, 21 bp, synthetic construct
  misc_feature 1..21: note = Synthetically prepared nucleic acid sequence
  source 1..21: mol_type = other DNA
agcggtggagtgcaaatagat

SEQ ID NO: 14 - DNA, 23 bp, synthetic construct
  misc_feature 1..23: note = Synthetically prepared nucleic acid sequence
  source 1..23: mol_type = other DNA
ttgctggacatcaacagtatcat

SEQ ID NO: 15 - DNA, 4149 bp, Staphylococcus aureus
  source 1..4149: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgtagaaaactttaattttttatctcctaattacactgaacaacaaaaaataggtaatttcttcagcaaactcgaccgccagattgagttagaagaagagaaacttgaactcttagagcaacaaaagcgtggatatattcagaagattttttctcaagatttaagatttaaagatgaaaatggaaacagttatcctgattggtctattaaaaagattgaagatatttctaaagttaataaagggtttactccaaatacaaaaaatgataaatactgggatgaattaaatgaaaattggttatctatagcaggtatgacacagaaatatttgtataaaggaaataaaggaattactgaaaaaggtgcatcaaagcatgtaaaagtagataaagatactctaataatgagttttaaattgactttaggtaagttagctatagtaaaagagcctatctatacaaatgaagctatatgccatttcgtatggaaagaaagtaatgttaatactgagtatatgtactactatttaaattctataaatataagtacttttggtgcacaggcagttaaaggagtaacattaaataacgatgcaattaatagtattatagtaaagttaccagtgatacaagaacaaaataaaatagcatactttttcaataaattagataaattaattgaaaaacaatcttctaaagtagaattattaaaacaacgcaaacaaggatttttacagaaaatgtttgtttaattcttataaagttctattatgtaaaatattaaatagagataacattatgaaagcgagcccaagacataaagtttttgaataaataaaaaagataatttctatcaaattaatatagaaattgtcttttttataaattttttgattatttttagctgattgagctgttacttttcttataataagtgctattagcacaaatcctagttctcttttggctttgtttattcctcttacggacattcgagtgaaacccattttaattttattagaagtaatttaggtttgaacccacctaaataaatatatgagttatttttttatgctacaaaatatattcagatttcaataatgacataaaataggcatctttatatttacctttagtgtagaattgctctttgagtaatccttctgttttaaatccttgtgactcgtatatatgcacagcttttttgttatctgtatcaacatatagataaattttgtgcatgtttaatatatcgaatgcataatttatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctctctgattcatctaataaatgcttatcaaataaatattgaagttccgttaaggattcatatggttcttcaaaccaataagacataatagaatattcattatttaattcatgaacaaaaagtaaatcactatactctaatgctcttagtttcataattccactcccaaaattttctcatatatttgcattataaatataaataacgaataagtcatcattcactgtgaatactctattttaacaattcaccacatactaattctcattttcttgttattctcgatttattactcttactatgaaacctataaaattctcacatttgtttgtattaagaataaatacgtcgatagtaacaataaaaaaataaataataaagcatccctcaccgtaaaagtgaaggatgctctagttttattgaaatatacatttcattttgttaaataattattaataatattttgaaaatcattattacgtgaaatcttcatagattttatcaagtatttctttgccttcaattgctgtgaagtgatgtaccaatctatttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgtggaatgttaaaaatcctacaaatgtttattcatctgcaggatttttaaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctaaatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataagtaataattaataatatcttggaaatcattgttaagtattgttgtaatacaatcgtcattcataaaatcttcatagtttttatcaagaatttcttcatcttcgatagatgtgaaatgattagctaacccttttataatttaagtgtaatttgtgaatctaaac

SEQ ID NO: 16 - DNA, 4353 bp, Staphylococcus aureus
  source 1..4353: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcatatcataaatgatgcggttttttcagccgcttcataaagggattttgaatgtatcagaacatatgaggtttatgtgaattgctgttatgtttttaagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgtagaaaactttaattttttatctcctaattacactgaacaacaaaaaataggtaatttcttcagcaaactcgaccgccagattgagttagaagaagagaaacttgaactcttagagcaacaaaagcgtggatatattcagaagattttttctcaagatttaagatttaaagatgaaaatggaaacagttatcctgattggtctattaaaaagattgaagatatttctaaagttaataaagggtttactccaaatacaaaaaatgataaatactgggatgaattaaatgaaaattggttatctatagcaggtatgacacagaaatatttgtataaaggaaataaaggaattactgaaaaaggtgcatcaaagcatgtaaaagtagataaagatactctaataatgagttttaaattgactttaggtaagttagctatagtaaaagagcctatctatacaaatgaagctatatgccatttcgtatggaaagaaagtaatgttaatactgagtatatgtactactatttaaattctataaatataagtacttttggtgcacaggcagttaaaggagtaacattaaataacgatgcaattaatagtattatagtaaagttaccagtgatacaagaacaaaataaaatagcatactttttcaataaattagataaattaattgaaaaacaatcttctaaagtagaattattaaaacaacgcaaacaaggatttttacagaaaatgtttgtttaattcttataaagttctattatgtaaaatattaaatagagataacattatgaaagcgagcccaagacataaagtttttgaataaataaaaaagataatttctatcaaattaatatagaaattgtcttttttataaattttttgattatttttagctgattgagctgttacttttcttataataagtgctattagcacaaatcctagttctcttttggctttgtttattcctcttacggacattcgagtgaaacccattttaattttattagaagtaatttaggtttgaacccacctaaataaatatatgagttatttttttatgctacaaaatatattcagatttcaataatgacataaaataggcatctttatatttacctttagtgtagaattgctctttgagtaatccttctgttttaaatccttgtgactcgtatatatgcacagcttttttgttatctgtatcaacatatagataaattttgtgcatgtttaatatatcgaatgcataatttatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctctctgattcatctaataaatgcttatcaaataaatattgaagttccgttaaggattcatatggttcttcaaaccaataagacataatagaatattcattatttaattcatgaacaaaaagtaaatcactatactctaatgctcttagtttcataattccactcccaaaattttctcatatatttgcattataaatataaataacgaataagtcatcattcactgtgaatactctattttaacaattcaccacatactaattctcattttcttgttattctcgatttattactcttactatgaaacctataaaattctcacatttgtttgtattaagaataaatacgtcgatagtaacaataaaaaaataaataataaagcatccctcaccgtaaaagtgaaggatgctctagttttattgaaatatacatttcattttgttaaataattattaataatattttgaaaatcattattacgtgaaatcttcatagattttatcaagtatttctttgccttcaattgctgtgaagtgatgtaccaatctatttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgtggaatgttaaaaatcctacaaatgtttattcatctgcaggatttttaaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctaaatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataagtaataattaataatatcttggaaatcattgttaagtattgttgtaatacaatcgtcattcataaaatcttcatagattttatcaagaatttcttcatcttcgatagatgtgaaatgattagctaacccttttataatttaagtgtaatttgtgaatctaaac

SEQ ID NO: 17 - DNA, 1100 bp, Staphylococcus aureus
  source 1..1100: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcgtatcacaaataaaactaaaaaataagttgtatataacttattttgaaattggttaagtatataatatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttat

SEQ ID NO: 18 - DNA, 1109 bp, Staphylococcus aureus
  source 1..1109: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtatcacaaataaaactaaaaaataagttgtatataacttattttgaaattggttaagtatatagtatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttattatgttttg

SEQ ID NO: 19 - DNA, 1159 bp, Staphylococcus aureus
  source 1..1159: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtatcacaaataaaactaaaaaataagttgtatataacttattttgaaattggttaagtatatagtatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttattatgttttgtattgtctgttattgtgcaaaatatttcaatgctatggcatattgtgagc

SEQ ID NO: 20 - DNA, 1125 bp, Staphylococcus aureus
  source 1..1125: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaggcgtatcataaataaaactaaaaaacggattgtgtataatatattttaaatataaaaaggattgattttatgttaaataaattagaaaatgttagttataaatcattcgataattacactagtgaagatgatttgactaaagtaaatatattttttggaagaaatgggagtggaaaaagctcattaagtgaatggttaagaagactagataatgaaaaaagtgttatctttaatactggttacttaaaaaataatattgaagaagttgaagaaatagatggtgtgaatttggttattggagaagaatctataaatcatagtgaccaaattaagcatttaaatagcgctataaatagtttagaaaattttattactcggaaaaatagtgaacttaagcattcaaaagaaagaatttacaataaaatgaatatcagactaaatgaagctagagaaagatttgaaataggtagtaatgtggttaagcagaagaggaatgctgacaaagatccagttaatgctttttatagttggaagaaaaatgctaacgatataattcaagagatgactattgaatctttagatgaattagaagaaagaataacaagaaaagaagtcttattaaataatataaaaacaccaattttagcttttgattataatgattttagt

SEQ ID NO: 21 - DNA, 818 bp, Staphylococcus aureus
  source 1..818: mol_type = genomic DNA
aatattggaagcaagccatagcagaatatgaaaaacgtttaggcccatacaccaagatagacatcatagaagttccagacgaaaaagcaccagaaaatatgagcgacaaagaaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcttatcataagtaatgaggttcatgatttttgacatagttagcctccgcagtctttcatttcaagtaaataatagcgaaatattctttatactgaatacttatagtgaagcaaagttctagctttgagaaaattctttctgcaactaaatatagtaaattacggtaaaatataaataagtacatattgaagaaaatgagacataatatattttataataggagggaatttcaaatgatagacaactttatgcaggtccttaaattaattaaagagaaacgtaccaataatgtagttaaaaaatctgattgggataaaggtgatctatataaaactttagtccatgataagttacccaagcagttaaaagtgcatataaaagaagataaatattcagttgtagggaaggttgctactgggaactatagtaaagttccttggatttcaatatatgatgagaatataacaaaagaaacaaaggatggatattatttggtatatctttttcatccggaaggagaaggcatatacttatcttgaatcaaggatggtcaaagataa

SEQ ID NO: 22 - DNA, 27 bp, synthetic construct
  misc_feature 1..27: note = Synthetically prepared nucleic acid sequence
  source 1..27: mol_type = other DNA
aacaggtgaattattagcacttgtaag

SEQ ID NO: 23 - DNA, 24 bp, synthetic construct
  misc_feature 1..24: note = Synthetically prepared nucleic acid sequence
  source 1..24: mol_type = other DNA
gagcggatttatattaaaactttg

SEQ ID NO: 24 - DNA, 24 bp, synthetic construct
  misc_feature 1..24: note = Synthetically prepared nucleic acid sequence
  source 1..24: mol_type = other DNA
gttgccatagattcaatttctaag

SEQ ID NO: 25 - DNA, 1081 bp, Staphylococcus aureus
  source 1..1081: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatc

SEQ ID NO: 26 - DNA, 1054 bp, Staphylococcus aureus
  source 1..1054: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaa

SEQ ID NO: 27 - DNA, 22 bp, synthetic construct
  misc_feature 1..22: note = Synthetically prepared nucleic acid sequence
  source 1..22: mol_type = other DNA
ttcttaagtacacgctgaatcg

SEQ ID NO: 28 - DNA, 24 bp, synthetic construct
  misc_feature 1..24: note = Synthetically prepared nucleic acid sequence
  source 1..24: mol_type = other DNA
taatttcctttttttccattcctc

SEQ ID NO: 29 - DNA, 24 bp, synthetic construct
  misc_feature 1..24: note = Synthetically prepared nucleic acid sequence
  source 1..24: mol_type = other DNA
tgataagccattcattcaccctaa

SEQ ID NO: 30 - DNA, 19 bp, synthetic construct
  misc_feature 1..19: note = Synthetically prepared nucleic acid sequence
  source 1..19: mol_type = other DNA
ggatcaaacggcctgcaca

SEQ ID NO: 31 - DNA, 37 bp, synthetic construct
  misc_feature 1..37: note = Synthetically prepared nucleic acid sequence
  source 1..37: mol_type = other DNA
cccgcgcgtagttactgcgttgtaagacgtccgcggg

SEQ ID NO: 32 - DNA, 37 bp, synthetic construct
  misc_feature 1..37: note = Synthetically prepared nucleic acid sequence
  source 1..37: mol_type = other DNA
cccgcgcatagttactgcgttgtaagacgtccgcggg

SEQ ID NO: 33 - DNA, 37 bp, synthetic construct
  misc_feature 1..37: note = Synthetically prepared nucleic acid sequence
  source 1..37: mol_type = other DNA
cccgcgcgtagttactacgttgtaagacgtccgcggg

SEQ ID NO: 34 - DNA, 24 bp, synthetic construct
  misc_feature 1..24: note = Synthetically prepared nucleic acid sequence
  source 1..24: mol_type = other DNA
caaattgtagcattcttgaactat

SEQ ID NO: 35 - DNA, 24 bp, synthetic construct
  misc_feature 1..24: note = Synthetically prepared nucleic acid sequence
  source 1..24: mol_type = other DNA
ctcccatttcttccaaaaaatata

SEQ ID NO: 36 - DNA, 29 bp, synthetic construct
  misc_feature 1..29: note = Synthetically prepared nucleic acid sequence
  source 1..29: mol_type = other DNA
gtcaaaaatcatgaacctcattacttatg

SEQ ID NO: 37 - DNA, 1256 bp, Staphylococcus aureus
  source 1..1256: mol_type = genomic DNA
gctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgta

SEQ ID NO: 38 - DNA, 23 bp, synthetic construct
  misc_feature 1..23: note = Synthetically prepared nucleic acid sequence
  source 1..23: mol_type = other DNA
gaggaccaaacgacatgaaaatc

SEQ ID NO: 39 - DNA, 756 bp, Staphylococcus aureus
  source 1..756: mol_type = genomic DNA
tgacattcccacatcaaatgatgcgggttgtgttaattgagcaagtgtatagagcatttaagattatgcgtggagaagcgtatcacaaataaaactaaaaaataggttgcgcataatataattagaaaggaattagacataaattaggagtccttcacagaatagcgaaggactcccattaaatatattatggtgtaaagaaatcacaaatcaatatatatacttaataccatatattaacttgtactattataaagtacgacatcagtattaggtatcactttgaacacatgaatttcattatcacttttattattcacaaaaaattttccaattctcaattactgaattatgtgtatacatgttgttaaaaattaataaaggatatttatgtttgtttaaagcatatcacaagtgatgcggttttttataaagatttacttgttagtgattttgataaaaatgcttaatactatttcaataatatgtatttaaaaattagattaatagtatttaacttcaaatggcctcgtataaactcatagcaaattaacgtaaatcaatgaaataaaatgaaaacaatttcaagaatacattataaacataaagtatacaaaaaataaatgagcgtatttgtttaaacgtatacactcatttttattaaattaatttattatattttacgattgttatttatgaaattaacaaattccatttttgatagtgaaattaaaagctttatcacttattattgat

SEQ ID NO: 40 - DNA, 771 bp, Staphylococcus aureus
  source 1..771: mol_type = genomic DNA
tgacattcccacatcaaatgatgcgggttgtgttaattgagcaagtgtatagagcatttaagattatgcgtggagaagcgtatcacaaataaaactaaaaaataggttgcgcataatataattagaaaggaattagacataaattaggagtccttcacagaatagcgaaggactcccattaaatatattatggtgtaaagaaatcacaaatcaatatatatacttaataccatatattaacttgtactattataaagtacgacatcagtattaggtatcactttgaacacatgaatttcattatcacttttattattcacaaaaaattttccaattctcaattactgaattatgtgtatacatgttgttaaaaattaataaaggatatttatgtttgtttaaagcatatcacaagtgatgcggttttttataaagatttacttgttagtgattttgataaaaatgcttaatactatttcaataatatgtatttaaaaattagattaatagtatttaacttcaaatggcctcgtataaactcatagcaaattaacgtaaatcaatgaaataaaatgaaaacaatttcaagaatacattataaacataaagtatacaaaaaataaatgagcgtatttgtttaaacgtatacactcatttttattaaattaatttattatattttacgattgttatttatgaaattaacaaattccatttttgatagtgaaattaaaagctttatcacttattattgataattttgactgcatc

SEQ ID NO: 41 - DNA, 680 bp, Staphylococcus aureus
  source 1..680: mol_type = genomic DNA
ttcagcaaaatgacattcccacatcaaatgatgcgggttgtgttaattgaacaagtgtacagagcatttaagattatgcgtggagaagcgtatcataagtagcggaggagttttttaccttgtgacttatcataaagtacgatgtttatgtaagtgattatcattatttaagcaggtttttcaaattaaataataacaagaataaaatgcacttagcgacattgaaatttattaatctagtaaactaatagatttatagaaaattttatttgcaaggggataattttgaaaagtagtattttctatctttccataatacattgtaattacaacggaggggatattgtgatgaagtgtatagataaaacgtgggttagctattataaagaattagctgataagttaacagattatcaaaataaacgttatgaattaattgaaatagtgaaggaagtatataaaaaaacgggaataaaattccctactttagcaagtgataatgtattgatggacatagatccttttacaatatttgcattatttaataaaaattccatgagagaaactaataaggtaaaaatattaacagaattagcttcggaattgaatattaagtccaaaattccgtcagtttttgacagtattccaacagtcaataatctgaatgctacatattataa

SEQ ID NO: 42 - DNA, 1119 bp, Staphylococcus aureus
  source 1..1119: mol_type = genomic DNA
gacattcccacatcaaatgatgcgggttgtgttaattgagcaagtgtatagagcatttaagattatgcgtggagaggcgtatcataagtaaaactaaaaaattctgtatgaggagataataatttggagggtgttaaatggtggacattaaatccacgttcattcaatatataagatatatcacgataattgcgcatataacttaagtagtagctaacagttgaaattaggccctatcaaattggtttatatctaaaatgattaatatagaatgcttctttttgtccttattaaattataaaagtaactttgcaatagaaacagttatttcataatcaacagtcattgacgtagctaagtaatgataaataatcataaataaaattacagatattgacaaaaaatagtaaatataccaatgaagtttcaaaagaacaattccaagaaattgagaatgtaaataataaggtcaaagaattttattaagatttgaaagagtatcaatcaagaaagatgtagttttttaataaactatttggaaaataattatcataatttaaaaactgacaatttgcgagactcataaaatgtaataatggaaatagatgtaaaatataattaaggggtgtaatatgaagattaatatttataaatctatttataattttcaggaaacaaatacaaattttttagagaatctagaatctttaaatgatgacaattatgaactgcttaatgataaagaacttgttagtgattcaaatgaattaaaattaattagtaaagtttatatacgtaaaaaagacaaaaaactattagattggcaattattaataaagaatgtatacctagatactgaagaagatgacaatttattttcagaatccggtcatcattttgatgcaatattatttctcaaagaagatactacattacaaaataatgtatatattataccttttggacaagcatatcatgatataaataatttgattgattatgacttcggaattgattttgcagaaagagcaatcaaaaatgaagacatagttaataaaaatgttaatttttttcaacaaaacaggcttaaagagattgttaattatagaaggaatagtg

SEQ ID NO: 43 - DNA, 24 bp, synthetic construct
  misc_feature 1..24: note = Synthetically prepared nucleic acid sequence
  source 1..24: mol_type = other DNA
cattttgctgaatgatagtgcgta

SEQ ID NO: 44 - DNA, 23 bp, synthetic construct
  misc_feature 1..23: note = Synthetically prepared nucleic acid sequence
  source 1..23: mol_type = other DNA
gaggaccaaacgacatgaaaatc

SEQ ID NO: 45 - DNA, 21 bp, synthetic construct
  misc_feature 1..21: note = Synthetically prepared nucleic acid sequence
  source 1..21: mol_type = other DNA
atcaaatgatgcgggttgtgt

SEQ ID NO: 46 - DNA, 26 bp, synthetic construct
  misc_feature 1..26: note = Synthetically prepared nucleic acid sequence
  source 1..26: mol_type = other DNA
aaacgaaaatactggtgaagatatta

SEQ ID NO: 47 - DNA, 25 bp, synthetic construct
  misc_feature 1..25: note = Synthetically prepared nucleic acid sequence
  source 1..25: mol_type = other DNA
ttgcctttctcaagtctttacaact

SEQ ID NO: 48 - DNA, 20 bp, synthetic construct
  misc_feature 1..20: note = Synthetically prepared nucleic acid sequence
  source 1..20: mol_type = other DNA
cgaggagaagcgtatcacaa

SEQ ID NO: 49 - DNA, 27 bp, synthetic construct
  misc_feature 1..27: note = Synthetically prepared nucleic acid sequence
  source 1..27: mol_type = other DNA
agttgccatagattcaatttctaaggt

SEQ ID NO: 50 - DNA, 27 bp, synthetic construct
  misc_feature 1..27: note = Synthetically prepared nucleic acid sequence
  source 1..27: mol_type = other DNA
aacaggtgaattattagcacttgtaag

SEQ ID NO: 51 - DNA, 26 bp, synthetic construct
  misc_feature 1..26: note = Synthetically prepared nucleic acid sequence
  source 1..26: mol_type = other DNA
ctattgttccacaaattatgattact

SEQ ID NO: 52 - DNA, 26 bp, synthetic construct
  misc_feature 1..26: note = Synthetically prepared nucleic acid sequence
  source 1..26: mol_type = other DNA
cttcttttcttgttattctttcttct

SEQ ID NO: 53 - DNA, 20 bp, synthetic construct
  misc_feature 1..20: note = Synthetically prepared nucleic acid sequence
  source 1..20: mol_type = other DNA
ctaaatcatagccatgaccg

SEQ ID NO: 54 - DNA, 20 bp, synthetic construct
  misc_feature 1..20: note = Synthetically prepared nucleic acid sequence
  source 1..20: mol_type = other DNA
atgctctttgttttgcagca

SEQ ID NO: 55 - DNA, 1073 bp, Staphylococcus aureus
  source 1..1073: mol_type = genomic DNA
tattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaggcgtatcataagtgatgcttgttagaatgatttttaacaatatgaaatagctgtggaagcttaaacaatttgtttatctaagtacttatttaataattgattgaactgtgattggcaccaggctgtctggtaaattgagaagttgggttttggagcgtataaatgatagaattaatataaaattcaatttgaggagtaggagattatgtcgaatataaaaacaacactagagacgtccgtaggactagaaaaagacaacgataagctatttgattatataactgaattagagattcaaaacacgcctgaaaaccgggaagcaaaagttgttattgaagaaaggttacataaagaatataaatatgaattagatcaaatgacaccagagtatggaatacaaaaaggcagtgttagaataggtcatgcagatgttgtaatatttcatgattctaaagataaatctcaagagaatattaaaataatagtagagtgtaaaagaaagaatcgcagggatggtattgaacaattaaaaacatatcttgcagggtgtgagtctgcagaatacggcgtttggtttaatggagaagatatagtatatataaaacgattgaaaaaagcaccacattggaaaacagtatttaatataccga

SEQ ID NO: 56 - DNA, 2031 bp, Staphylococcus aureus
  source 1..2031: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagtgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgttttcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcgtatcacaaataaaactaaaaaatagattgtgtataatataaaaggagcggatttatattaaaactttgaattcaaaaattattgaaagggaagctaccttagaaattgaatctatggcaactaatacattgaaaataaacccggatattaattcaaacgatacaaaaatgtctttcgatggagaattggaagtgtatgattctgaaaatttgagtaaaaaaaatttcgttggaaaaatacaagttcaagttaaaggaaaggaagtagctaaaagaggaggtaaggttattcatcgaagtaatgtcaaaatgaatgatttaaaggcataccaacgagaaggtggtgtgtattactttgtcgtgtatttaatcgttgagaataaaaaagttgttgagaagcaggtttatggtaaacaattacatcaattagatttacaatttttactgcaaaaaaagcagaaaagcgtcactataaaaatgtatgaaattgaaaatgaaaaaattttatataataattgcgtaaaatatataaatgaaaagagattacaaaatcaagtaggtcaagttaaagtaaagaacatagaaaaagctttatcatatatagctacacctgaaaatattgtgatggatcatagaggtttaccattaaatgatttttatggctatataaaaattaattcatctgaattagatgtaactataccagatggagtattaagtatggaaaaagtaaaaagagtgaacaaaaagcagataataaaagaaggcaagttattatttgaaggtatggtaggtattgaaacttcaaaggagtccatctctataactatagatgatattttcaaaattcaaacatttgaaagtgataacaaaagtatatatacaatgttaccatttaaaaaattaaatatagccgaacaatcttttaatgttataaacgaattgtcgaaaggcggcgaattctttttggatcaaatccaattagtaatccaaccttttgaaattaatattattgaaataaaagaaacaataaataagttgaatattaaattatcagagtatagtaacttgctttcgtttgatgtgagtctaaaatctacagaatttgataagcagatgaatgagataaaaggtttattagaattattggaatataaaaattttaaagattttaaaatgcataataatggatactataaaatgaagttttgtggaaaatttatattattatttaaagacaatacatcattgtataatgtctactctaatgactttgtagatagatttgaggctgttacaaaagaaagagttgtacagatgccaattgtttacacattaacaagagatatgattgtagatgtactgaattttgatataaatgttattaaagaatgtattgaatcagataaaattgctattcaatctgacattaaatgggagaaattaaataactttgcattagaattaatcgcagcctatgatgaaactcaaagaaccgatttattagaattagctgaatatgtattaaataatttgctgaattttgataatgataaaatatttatgaacttaa

SEQ ID NO: 57 - DNA, 22 bp, synthetic construct
  misc_feature 1..22: note = Synthetically prepared nucleic acid sequence
  source 1..22: mol_type = other DNA
cgtggagaggcgtatcataagt

SEQ ID NO: 58 - DNA, 22 bp, synthetic construct
  misc_feature 1..22: note = Synthetically prepared nucleic acid sequence
  source 1..22: mol_type = other DNA
gctccaaaacccaacttctcaa

SEQ ID NO: 59 - DNA, 23 bp, synthetic construct
  misc_feature 1..23: note = Synthetically prepared nucleic acid sequence
  source 1..23: mol_type = other DNA
gccaaaattaaaccacaatccac

SEQ ID NO: 60 - DNA, 30 bp, synthetic construct
  misc_feature 1..30: note = Synthetically prepared nucleic acid sequence
  source 1..30: mol_type = other DNA
aatctttgtcggtacacgatattcttcacg

SEQ ID NO: 61 - DNA, 30 bp, synthetic construct
  misc_feature 1..30: note = Synthetically prepared nucleic acid sequence
  source 1..30: mol_type = other DNA
cgtaatgagatttcagtagataatacaaca

SEQ ID NO: 62 - DNA, 23 bp, synthetic construct
  misc_feature 1..23: note = Synthetically prepared nucleic acid sequence
  source 1..23: mol_type = other DNA
gccaaaatcaaaccacaatccac

SEQ ID NO: 63 - DNA, 27 bp, synthetic construct
  misc_feature 1..27: note = Synthetically prepared nucleic acid sequence
  source 1..27: mol_type = other DNA
attgctgttaatattttttgagttgaa

SEQ ID NO: 64 - DNA, 21 bp, synthetic construct
  misc_feature 1..21: note = Synthetically prepared nucleic acid sequence
  source 1..21: mol_type = other DNA
tattgcaggtttcgatgttga

SEQ ID NO: 65 - DNA, 22 bp, synthetic construct
  misc_feature 1..22: note = Synthetically prepared nucleic acid sequence
  source 1..22: mol_type = other DNA
tcaccgtctttcttttgacctt

SEQ ID NO: 66 - DNA, 23 bp, synthetic construct
  misc_feature 1..23: note = Synthetically prepared nucleic acid sequence
  source 1..23: mol_type = other DNA
cagcaattcwcataaacctcata

SEQ ID NO: 67 - DNA, 26 bp, synthetic construct
  misc_feature 1..26: note = Synthetically prepared nucleic acid sequence
  source 1..26: mol_type = other DNA
caggcatagctatatatgataaagca

SEQ ID NO: 68 - DNA, 26 bp, synthetic construct
  misc_feature 1..26: note = Synthetically prepared nucleic acid sequence
  source 1..26: mol_type = other DNA
atgcctttaaatcattcacattgaca

SEQ ID NO: 69 - DNA, 4340 bp, Staphylococcus aureus
  source 1..4340: mol_type = genomic DNA
cttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataacaaaacagtgaagcaatccgtaacgatggttgcttcactgttttattatgaattattaataagtgctgttacttctcccttaaatacaatttcttcattttcattgtatgttgaaagtgacactgtaacgagtccattttctttttttatggatttcttatttgtaatttcagcgataacgtacaatgtattacctgggtatacaggtttaataaatttaacgttattcatttgtgttcctgctacaacttcttctccgtatttaccttcttctacccataatttaaatgatattgaaagtgtatgcatgccagatgcaatgatacctttaaatctactttgttctgctttttctttatctatatgcatatattgaggatcaaaagttgttgcaaattggataatttcttcttctgtaatatgaaggctttttgttttgaatgtttctcctactataaaatcatcgtatttcatatatgtctctctttcttattcaaattaattttttagtatgtaacatgttaaaggtaagtctaccgtcactgaaacgtaagactcacctctaactttctattgagacaaatgcaccattttatctgcattgtctgtaaagataccatcaactccccaattagcaagttggtttgcacgtgctggtttgtttacagtccatacgttcaattcataacccgcttcttttaccatttttacttttgctttagtaagtttggcatcttcagtgtttactattttagcattacagtaatctaaaagtgttctccagtcttcacgaaacgaagttgtatggaatataactgctctgttatattgtggcatgatttcttctgcaagtttaacaagcacaacattaaagcttgaaatgagcacttcttgattctgatttaagtttgttaattgttcttccacttgcttaaccatacttttagaaagtgctagtccattcggtccagtaataccttttaattctacatttaaattcatattatattcatttgctatttttactacatcatcgaaagttggcaaatgttcatctttgaatttttcaccaaaccaagatcctgcagaagcatctttaatttcatcataattcaattcagttatttccccggacatatttgtagtccgttctaaataatcatcatgaatgataatcagttgttcatcttttgtaattgcaacatctaactccaaccagtttataccttctacttctgaagcagctttaaatgatgcaattgtattttccggagctttactaggtaatcctctatgtccatatacagttagcatattacctctccttgcatttttatttttttaattaacgtaactgtattatcacattaatcgcacttttatttccattaaaaagagatgaatatcataaataaagaagtcgatagattcgtattgattatggagttaatctacgtctcatctcatttttaaaaaatcatttatgtcccaagctccattttgtaatcaagtctagtttttcggttctgttgcaaagttgaatttatagtataattttaacaaaaaggagtcttctgtatgaactatttcagatataaacaatttaacaaggatgttatcactgtagccgttggctactatctaagatatacattgagttatcgtgatatatctgaaatattaagggaacgtggtgtaaacgttcatcattcaacggtctaccgttgggttcaagaatatgccccaattttgtatcaaatttggaagaaaaagcataaaaaagcttattacaaatggcgtattgatgagacgtacatcaaaataaaaggaaaatggagctatttatatcgtgccattgatgcagagggacatacattagatatttggttgcgtaagcaacgagataatcattcagcatatgcgtttatcaaacgtctcattaaacaatttggtaaacctcaaaaggtaattacagatcaggcaccttcaacgaaggtagcaatggctaaagtaattaaagcttttaaacttaaacctgactgtcattgtacatcgaaatatctgaataacctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccatattttatgtgttgttgtttgtggtgcattagcagcaaacattcgtggtgaaagtgatgctgttttagcatttgataatttgaatgttgctgattgagttggtttttgtgttgaggcttgttgtgcatctacgtcgcgcgtttcgtttactgttgttgaaactgctgtagatggtttgttagatggtgcgttggatgttactttgttgtttgtatcatcagctgttccctgtactgatgctgatacgttggttgccgcttcttcaccagattgatgagccgcttcctttgttggcgttacttgttgcttatctgttgttgctgcttgtgctttcattggtgtatgctgctcagccgcgttcgagacatgccataatcccatcacactaaaaagcaaaacgagccatacacttgttttaagtaataaagctttcatttcgttccctccattaactgtcatacaccaatatattatcataatccttatattaaaatgtttgattttatgtaaattatttataaattacatttaatatcgaattattttacacatttcaatcaatccgattcaactcaacaacagccatttcccaccaacaacataaaaaagcacccgataaagtcgtttcacagcagtgcggaactttatcggatgactctcatttatattgtatatattggtgttggaatagctttatccgtatcacatagcaatacttctttttccgtatcaatatcgtgttcgttcaatacttggccaacactcatacgcgccaaatctttcatattattatcttgtgataaatgcgataagtaaatacgtttcgtgttacctgtaatcacgtctgtcatcgcatgacccgcatcctcattagatacatgacccatatcgcctaaaatgcgttgtttcgtcttccatggataacgacacattctcaacatatcgacgtcatgattactctcaaaaataaatgcatcgctgccacgtatcatacctttcatacgatcagacacgtaacccgtatctgttaaaatcgtaaacttcttatagttattatggaaaatataaaattgcggatctatcgcatcatgtgacacgttaaacgattca

SEQ ID NO: 70 - DNA, 29 bp, synthetic construct
  misc_feature 1..29: note = Synthetically prepared nucleic acid sequence
  source 1..29: mol_type = other DNA
atttcatatatgtaattcctccacatctc

SEQ ID NO: 71 - DNA, 29 bp, synthetic construct
  misc_feature 1..29: note = Synthetically prepared nucleic acid sequence
  source 1..29: mol_type = other DNA
caaatattatctcgtaatttaccttgttc

SEQ ID NO: 72 - DNA, 29 bp, synthetic construct
  misc_feature 1..29: note = Synthetically prepared nucleic acid sequence
  source 1..29: mol_type = other DNA
ctctgctttatattataaaattacggctg

SEQ ID NO: 73 - DNA, 22 bp, synthetic construct
  misc_feature 1..22: note = Synthetically prepared nucleic acid sequence
  source 1..22: mol_type = other DNA
caagctccattttgtaatcaag

SEQ ID NO: 74 - DNA, 27 bp, synthetic construct
  misc_feature 1..27: note = Synthetically prepared nucleic acid sequence
  source 1..27: mol_type = other DNA
cactttttattcttcaaagatttgagc

SEQ ID NO: 75 - DNA, 27 bp, synthetic construct
  misc_feature 1..27: note = Synthetically prepared nucleic acid sequence
  source 1..27: mol_type = other DNA
acaaactttgaggggatttttagtaaa

SEQ ID NO: 76 - DNA, 23 bp, synthetic construct
  misc_feature 1..23: note = Synthetically prepared nucleic acid sequence
  source 1..23: mol_type = other DNA
gccaaaatcaaaccacaatcaac

SEQ ID NO: 77 - DNA, 23 bp, synthetic construct
  misc_feature 1..23: note = Synthetically prepared nucleic acid sequence
  source 1..23: mol_type = other DNA
accacgaatgtttgctgctaatg

SEQ ID NO: 78 - DNA, 25 bp, synthetic construct
  misc_feature 1..25: note = Synthetically prepared nucleic acid sequence
  source 1..25: mol_type = other DNA
acgcaattgttaaaaatatgtgtcc

SEQ ID NO: 79 - DNA, 20 bp, synthetic construct
  misc_feature 1..20: note = Synthetically prepared nucleic acid sequence
  source 1..20: mol_type = other DNA
tggcaatgcaattcgtgaac

SEQ ID NO: 80 - DNA, 22 bp, synthetic construct
  misc_feature 1..22: note = Synthetically prepared nucleic acid sequence
  source 1..22: mol_type = other DNA
ttccactcccaaaattttctca

SEQ ID NO: 81 - DNA, 25 bp, synthetic construct
  misc_feature 1..25: note = Synthetically prepared nucleic acid sequence
  source 1..25: mol_type = other DNA
aatcgagaataacaagaaaatgaga

SEQ ID NO: 82 - DNA, 20 bp, synthetic construct
  misc_feature 1..20: note = Synthetically prepared nucleic acid sequence
  source 1..20: mol_type = other DNA
ttggcgaacagttgctagtt

SEQ ID NO: 83 - DNA, 26 bp, synthetic construct
  misc_feature 1..26: note = Synthetically prepared nucleic acid sequence
  source 1..26: mol_type = other DNA
tctctttataatttgagcttggttga

SEQ ID NO: 84 - DNA, 24 bp, synthetic construct
  misc_feature 1..24: note = Synthetically prepared nucleic acid sequence
  source 1..24: mol_type = other DNA
tggaaaaagtaaaaagagtgaaca

SEQ ID NO: 85 - DNA, 24 bp, synthetic construct
  misc_feature 1..24: note = Synthetically prepared nucleic acid sequence
  source 1..24: mol_type = other DNA
tggttatagaaatggactcctttg

SEQ ID NO: 86 - DNA, 22 bp, synthetic construct
  misc_feature 1..22: note = Synthetically prepared nucleic acid sequence
  source 1..22: mol_type = other DNA
acccggacattaattcaaacga

SEQ ID NO: 87 - DNA, 46392 bp, Staphylococcus aureus
  source 1..46392: mol_type = genomic DNA
aaaatatcaaaaactgcaaaaaatattggtataataagagggaacagtgtgaacaagttaataacttgtggataactggaaagttgataacaatttggaggaccaaacgacatgaaaatcaccattttagctgtagggaaactaaaagagaaatattggaagcaagccatagcagaatatgaaaaacgtttaggcccatacaccaagatagacagcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgccatcttccgaaggattggcccaagagttgaaccaacgcatgacccaagggcaaagcgactttgtattcgtcattggcggatcaaacggcctgcacaaggacgtcttacaacgcagtaactacgcactatcattcagcaaaatgacatttccacaccaaatgatgagggttgtgttaattgagcaagtgtacagagcgtttaagattatgcgtggagaagcgtatcataaataaaactaaaaaataggttgtgtataatataaaaggagcggatttatattaaaactttgaattcaaaaattattgaaagggaagctaccttagaaattgaatctatggctacaaatacattgaaaataaacccggacattaattcaaacgatacaaaaatgtcttttgatggagaattggaagtgtataattctgaaaatttgagtaaaaaaatttcgttggaaaaatacaagttcaagttaaaggaaaagaagtacctaaaagaggaggtaaggttattcatcgaagtaatgtcaatgtgaatgatttaaaggcataccaacgagaaggtggtgtgtattactttgtcgtgtatttaattgttgagaaccaagtttatggtaaacaattacatcaattagatctacaacttttactgcaaaaaaaatagaaaactgtcactataaaaatgtgtgaaatttaaaatgaaaaaaattatataataactgtttaaaatatataaatgaaaagcgattacaaaaccaagtagatcaagttaaagtaaagaaaaaagaaaatgctttatcatatatagctatgcctgaaaatattgtgatggatcacagaggtttaccattaaatgatttttatggctatataaaaattgattcatctgaattagatgtaactataccaaacgaagtattaagtatggaaaaagtaaaaagagtgaacaaaaagcagataacaaaagaaggtaaattattatttgaaagtatggtaggtattgaaacttcaaaggagtccatttctataaccatagatgatattttcaaaattcaaacatttgaaagtgataataaaagtacatatacaatgttaccatttaataaattaaatatagccgaacaatcttttaatgttataaacggattgtcgaaagttggctaattcttttttgatgaaattcaattagtaatagaacttttgaaattaatattaatgaaataaaagaaacaataaataaattaaaaattaagttatcagagtatagtaacttgctttcgtttaatgtgagcctaaaacctacagaatttgataagcaaatgaatgaaataaaaggtttattagaattattggaatataaaaaatttaaagataatacatcattatataatgtatattctaatgaatttatggatagatttgaagctattacaaaagaagaatctgtacaaatgccaattgtgtacacattaacaagagatatgattgtaaatgtactaaattttgatataaatgttattaaagaatgtgttgaatcagataaaatcgctattcaatctggcattaaatgggagaaactaaataattttgcattagaattaatcgcagcctatgatgaaactcaaagaatcgatttattagaattagctgaatatatattaaataatttgctgaattatgataatgataaaatgtttatgaacttgatcaaccaagctcaaattataaagagaagaaaaagtatggtagattaaaaattgttaagcgacttatttgaattgaaagataaattagttgatatttataaggttgatatgaagaaatttcaaaaaaattctggggaaagtgaaagggcagcttatagtttcaatggtattgcttttgatatattatgtgtcttattaaataatatgacagatgataatataccaaaagcaattacggatgaagatgtaaggctgagaaaagatgcgataaaaaatattgatattacccaatatgcgaattttattaaaaatgttaagaatgatatagataaaattgaatttaaaccattaaagttacatatacatgcacaaaaagcataccaagatgtaaaaaggtgtttgattcaggcagtaaaataaacgataagttacaattagcttctgaagtaatgagtcaattacctatagataaacaagtggaattaagtgacaaaattgcacttgctataaatgaagttgtttactcaacattttcagaaagtaaatataatgaacgaattgatgaacacaatcaaacgagtgaaaagtcaagtcaatatgattttcgatataattattttgtaaatcaattaatagataatgtaataacagaagatatagatgaagagaaagcatttatatatgatagaaatgaacaattagattggttattagttaagatactaaataaagttcaaagagctgcggatggatggggaatgagaaatgctaaacttcatcacagaacacaaacatttagagatatggaacattatttcctcagagatatatcgaaaatgatagataaagagcttaacaaaaaggaaaacttaaataagcaatctaaaaaatatgacttcgataataagataaaggtcataaatgacaatattgagttttttaatgaagatgaacaaattagaaaacatttactagagttacgcaagcgattagactttcattctgcagcagtattagataagaaaaatcattcacacatagggaatgaaatgttccaatattttatggatgatatgcagcaagttgtaacattgatagaccgatacattaataagactgaacattataatgatatgaaaaatggctatattaatgaatataaatcattttataatgcttataataatgaaattgaagtgactaagacgcacgaaaataacatcccaatggcaatgttaatgaagcaatttaagaatcaactgttaaaaagaaggaataaccgaaaaagctctaattgaagttaagtagaaatttaataaataattggtaagctcaatccctcatataaagggattgagcttatttgcttggtgggaatttttttgaatttttttgccacttcatcaacgacctctatttagtattaagattgtcttgtcaaaacgttttgaacaaattttaagttgaaagtgagcgcgccactttcaaccataaaggagaagatttacatggagacaagagacaaactgatgtcgttgactcagagtaacaaaatacagcaatggttaatggacaaatcatctaaccaagatgaggttcaacaattgcagcaaccaattcagtcagccagctagatcaacaatataatgcacttttagctgatgaaaaagctaagttagaccaatacgtggaagtacatcaaggattggaatcattaaaggaagagattgaatcagaacctattacgattaatatcgataaattacccgatatcaaagcaacaatgcttgaaagagccaagaatgatgaacattctgataagatcgaaaagctatttgaaaggttagaacaggcattaagtggtacgaatcgattatatacgcaattatcgttgattggtacacgaacacatagaattacaactaaaaattttaatcttcaaggcttacctaaagcagttcaacatacgattttaccttcaaaatttaagaaggtgtatacaatcgatttcaactcatttgaaccttcggttgttgcgtacatgactcaagattcaaaactgattgactttttaaatcagaaagacggactgtatgacgcattgctaagtgaattaggcttatcagatgagctacgagtatttgttaaacgtgcatttattggttcgtttctatttggcggcaacttccatagccctaaattcaagttgaaacattatgtaagtgaagtgcaatggttggatgcggtcagccaatttacaaaagtcattgaacttaagaaacaaatcgaagagcataaaaccatgcctatgccttacggcattgaacatgatatgagcgcattttaaggtagcagtattatggcattttatgtacaaactgtagcgagttatattttcaaacacattctattggaagtttataaagcgcagtgtacacaaaagagctttcaaatcattttgccaattcatgatgccattatgattgagtgcgacgatgaagaaacagcccaaagtgtagcccaactaatgaaaagtaaagctaataaattgtttaatggtgagtttgcgcacgtgacagtggaagcattaggaggtgtaagcaatgaataatgatagaggtcaaagtttacaaattccaaaatgcacctcactaacagaaggctatatatacgttgcttcattatattctgtaatccaaacagatttcaatggtgatgttaaacaccagtttacgtatgaaataaagatagatcagaaagttatttatgtgaatcgtagtatttcaaaagatgctaccgataagcagttatcaattactgattggcttaaacgtcatagtaattatagttccactcatgagaattataacccttacattgatagaaagcatttaatctatgtgggtgtcaatataacggaaactattatgttcaagatgtagcaccattagatggatttggaggggtattgtaatgaatcatattttagaaatgttaataaaattattaaaagtgggtatggaggcaatcgaccgtaaaggtctgattgccatcctaacaagtagtattggaaatgatgaaatggatgattctgaacaagctgtaatggtgtataacgagcttatcgataaactacagcttaacattcctaaagatgtcgactatagacctaacatatacagttattttggtattcaaaaaaagccaaatgacacaatattagtggaaatgatgataagtatttttaatatcaagcgttttgattcagagttgtttgttttcaaagataaaggttggcaaaaggtaaatgaagatgaattgcaaggactgatatctaaaatgatacaagtgttgctagttgattataagccctcgttaagtattctaaagaatgtagtggttggcttacaaaattcaatagatgtagaagaactagttgagaataaacagtccattgcatgcggtcataacatgtttaatctagacgcatttgaagtggttgaaaattctataaagattttctccgcaacacggttgaatttagagttggttaaaagtgatgtcataactgaaaaaacacctccacattttaatcgatacatgttagagtttgctaattttgattctgatttacagtactttctttttcaacatatagcggtgcttcttacagctaacacaaagtatcgcagagccttactattatatggtggatctaagaatgggaaatccgtagttattaatttagttaggtcatttttctacagcgaagacattgtgtctaaagcacttaacgagctttaaggccgttttgacaaagagagcttggtgggtaaaaaacttatggcaagtgacgaaattggggaatcaaggattcaagaaaagattgtaaatgacttaaaaaagttagtatctgttgaaccagtacacgttgatcgtaaaggtaaaacacaagttgaaactactttggatttaaaacttgcttttggtacaaatgcaagacttaattttccatctgctcatgcgaaggcactagagcgtcgaattgctgttattccatgtgaatattatgttgaaaaagctgaccctgacttaattgaaaagttacaggatgaaaagaaagaaatctttctttacttgatgtatgtgtataagcaaattgtaaaaaatgatatcgagtacctccaaaatgatcgtgttactgaaatttctcatgattggttaaattttggatatgaatttgtttctagtaaatcagcaagtattgaaaatcagaaagcgtgtattaatttactcagaaaacttataaaaatcaaaccaggatcacgaatcaaagtatccgagttaaatacaagttattaatgaagaaataaaggtgagttctcaggttattaaacagttaattcaagcaaactttgatactcaaaccaaactatacaatggctacgattattggattgatttaggttggaaagaagccgataaaaaagagattcatgatatttcggaaaaagataatattatttcattagataaaaatgaaaatataacagacgatgaggcattagatgaagagaatttagactttgattgggaggactttgacgatgaataatgaacaaattgaagcatttgtagaagtgtttgtgcctatcatagaagaacgtatcaataaaggtaagtactcatatgattaagtggtaaaagtgataaaaagaaatgaaattataaatatatattatccatatgatgttacaagaccgatggtctgtagcaataatctaataaaaggagcggtacattatgagtaagaaaattgcactgtactcacgtgtaagtacatctgagcaatctgaacgtggttattcaatccatgaacaagagcaagtgctcatcaaagaagttgtgaaaaatttcccgggttatgactatgagacatatactgactcaggcatttcaggtaaaaatattgaaggtcgtccggcaatgaaacgtctattacaagatgttaaggataataaaattgaaatggtattaagttggaaattgaatcgtatttctcgctcaatgagagacgtgtttaatattattcatgaattcaaagagcttgacgtaagttatcaatcgatttctgagaatattgatacatccaatgcttctggagaagtactcgttacaatgtttgggttgataggatcaatagaacgttcaacactgatcagtaatgtcaaaatgtcgatgaatgctaaagcacgtagtggagaggctattacagatagagtgctcggttacaagttgaaactcaatccacttacacagaaaaatgatttggttatcgatgaaaatgaagctaatatcgtacgtgaaattttcgatttatacttggatcataataaaggactcaaagccattacatcagtactgaatcaaaaggggtatcgtactattaattaaaagccattttcagtttatggtgttaaatatattttgaataatccagtctataaaggttatgtcagattcaataaccatcaaaattgggctgtacagcgaagaagtggcaaaagtgataaaaatgatgtgatattggtcaaaggtaaacatgaagccattataagtgaagaggtatttgaccaagttcatgaaaaattagcttctaaaagttttaaaccgggcagacctattggtggagatttctaccttcgtgggctcaataaatgtccagagtgtggcaataatatggtatgtcgtcggacttattataaaacgaaaaagtcgaaagaacggacaatcaaacgctattacatttgttctttattcaatcgttcagggagttctgcctgtcatagtaatacgatcaatgctgaagtcgtcgaacgtgtaatcaatgttcatctaaatcgtattttgtctcaaccggatgtgattaaaaagattgcgtcaaatgtgatagaaaaactgaaacaaaagcatagtaaacaaacagaaataaaatatgatattgatagtctagaaaaacaaaaagcaaaagttaaaacacaacaagaacgattattggaattgttcttagatgatgaaatggacagtgaaattttaaaagctaaacaaagtcaaatgaatcaacagttagaagtattagaccagcaaattaaagaagcaaaacaagcatatcaatcacaagctgaaatacccaattttgataagttaaaaggacgactcattttaatgataacacgattcagtgtgtattttagaaaagccacacccgaagctaaaaatcaacttatgaaaatgttaattgattcgattgaaattacgacagataaacaggtgaaactagtacgatataaaattgatgaaagtcttatccctcaatctttgaaaaaagattgggggtcttttttatgcccaaattccaattcgaaatatatggtcaaaatgattatttcatcaaccaaattaccacttttaccacttagttattagtgaaaaaagtgagagaaatgaaataaaaatcaaatatatatcatctaaatgatatatcacatgtatacatcaaccaaatacattaggaggttataacaatgacattagaactacaactcaaacactatataaccaatttattcaacctaccaaaggatgaaaagtgggaatgtgaatctatcgaggaaatcgctgatgatatcctacctgaccaatatgtaaggcttggggcactcagtaataaaatacttcaaacctatacctactactctgatacacttcacgaaagtaatatctaccctttcattctctactatcagaaacagctcatagccatcggctatatcgatgaaaatcacgatatggatttcttatacctacacaacaccatcatgccacttttggatcaacgatacttactaacaggaggacaataatatgcataaatatatcaaaattacacaattagtaatcacaatactaagtgaaatcatcatttggatgaaagagtcagaacgaaaggaagtctcttatgaatagatatatcacccgtggtatcgccaacaacttacctactgcattacaacatcaattatggcaatttgtagcacaacgtgaaaacgaacagtccaaggaactagaagcaatagattattttcatgtattccagttcaacatgcacaataatcaattatatgtaaaacacaaacaagaacgtcctgagtatgtcaaaattcataaagcgaattattcgaaagatctcaatattaataaggtctacattatccgagaatatgatgtagactttttcttactatgtcatgttactacccgaagaatattaataggaggcatcaatcatgcagaatatacaatgtacattagaaactgaagcaatatttagtgacgataatcaacatcgttacttactcaaaaaacatgggacaatgataaaaagataatttcagttataacttgttatccaaattttgaaggaacgaaaaaattagatttaacgactcaactaattatgaacaaagttgtagaaatggatgaatttggttccatcaattttgtgaatctatactctaatattacaacaccgattaatttcaaacatttagaaaccctctatgatgcctatgataagcatacagatattcaaattatgaaggcagccaaggagtcagatgaagtaatagaaatgttgaaaccacataagaagaaagtgaaacaactcattaaccccgaaagctcgtcgcaagtggatatataaacaaaacactatttaaaataattatacaaaagcgaaggtatttaatgtattttaataagaaaaagcaagaaaaaattgactaatataaaaattacattcgagaaattatattaaacggaaaacaagtgatttgttgcataaatttataaaagtagtgaaattatgagaacgtatagctgtattgattgctacatttattttgatgacaatattatagatatcgttgttgtatggagctatggcgataagtatatttggtggaaatgatgaaatgttaaataaagaacttaattcactgcaagaagcgatatgaaagtatgttaagggagtaaaatatgacattaccaaatttattaaattattattaatgcaatttttatgtgtatttcttgtagtacaagtaggacttgtatctggaggtagtgttctacttgcagtagtatatcttgaaattttagggtcttattttaaaaggatttttacaattaaatggccactcggtaaagatgtagcaaaaattatagaaaattactttggtgaacctgtataaatcagagaataattattaaaaatagaacatttcctacctttcatagtaggaaatgttctatttaatttatattttttctaacgaaactagaatacacctactttgataacttaattaacataagtagaggtatagtcgtattttgtcactttccaatatttacaaactatcttcaaagtcaatgctttatctgaaaaaaattttcaaagattttcttaaaatttacgaaggaatgacttattgtgaaaaacttgttttttaacatataacttaaaggagataatatattggattgtgtataccgattgtacgaaagcagatcatggataagtacacccaaagtaattaagtatgattataaatcgaaagttataatattgaacaagataaaagttaaaatatgtattttatgtatgatattttggctgatatttattatattgctccaaaattaaaccgttacttttagcaatatcttaaattttaatcataaaacatcatgcaaataaacgaagtaagttgttaatgaattttgatcgctttgaaattaatgaaaatcgatcaacaataacaatcgtatcaatcgtcacacgagcattaaatgaaaaatctaaatacaatcaaaaacttttaaatctatattgatttaatttcaatgtttgattttcaattaatttaacaattaatttgggagatctatatcaaagacattaataaaatgaaattaaagatacccaataaatagaaccacagtataaattatatcagtatgcttatataatttttgaaatctttaaacaaatgaagtaataattgagaaaagtgtagttaaattatttttcttgaaattatttgttacatagcatttcgatgtaaaattcactttttataagtaaatttaaaaagagtttgcaaaatatacaggggattatatataatggaaaacaagaaaggaaaataggaggtttatatggaaagtacaattgatttatctgaattattaggaagagtaagaaaaaatatgaaattattaataatattaccacttttaggtctgctaattagtgcaataatttcttttttctttttagatgtaaagtaccaagcttctactcaaatattagtgaatcaaaaaggaaatgactcacaaattatggcacaagaagttcaaagtaatattcaactagtaaacacatattcagagatagttaaaagcccacgtattctagataaggtttctaaagaattagatgataaatattcacgtagtgagatatcaagtatgttaaccgttaccaaccaagcagagtctcaagtattaaatattgatgttgaaagtaaaagtggtagtaattcagaaaaaattgctaataaaatagcagaagtatttagtgatgaagtgcctgatattatgaatgttgataatgtttctgttttatctacagcggataatacaggaaaacaagttgcaccaaaacctatggttaatttagttgttggtttagtaataggattggttatcgcattattaataatttttataaaagaagtgtttgataaacgtattaaaacagaagaagaagttgaaaatgaattggtgatacctgtactgggttcaatccaaaaatttgattaaggagggttttatctatggcaaaaaagaaaagcactatatcaccactatatgttcatgataaacctaaatcgacgataagtgaaaaatttagaggtattcgatcaaatatcatgttttctaatgctgaaaatgaaataaaaagcttgttgataacttctgaaaaatccgcatctggtaagagcatactttcggcaaacattgcagtaacatatgcacaggcaggttacaaaacattaattatagatggagatatgagaaagcctacacaacattatatctttgatttgcctaataatagtgggctatctaatttaattattaataaaactacatatagcgattcaattaaagaaactagagtagagaatttaaacgttttaacagcaggaccaacaccgcctaatccttcagaattaattgcttctagcaaatttgcaactatatttaatgaacttttgaatcattatgacttcattgtgatagacacacctccgattaatacagtaacggatgctcaagtatatgcgcgaatagtaaaaaattgtgtgcttgtcatagatgctgaaaaaaataataagagtgaagttaagaaagcgaaaggtttacttactaaagccggtgggaaagtattaggcgcagttttaaataaaatgccaattgataaaaattctagctactattactactatggagaagattaagaatggttgatatacataatcatattttggttgatgtagatgatggaccaaaaagcattaatgaagcaattgagttgcttaaacaagcccaaagtgaaaatgtaacagatatagttgcaacacctcaccatcttcataagagatatagtaatgatattgaaaaagtgaaaataaaattaaatgaattaaaaaataactcagaaataaaaaaattaggacttaatttatatgttggacaagagattcgtataacggatcaaattatagaaggtataaaaaataaagagattgaaggaattaatgaatctaggtatttgttaatagaatttcctagcaatgagattccttattatacgaatcaattattctatgagttacaaacaatgggatatattccaatcattgcgcatccagaacggaataaagcgatagttcaaaatttagatttactatttgaattaataaatggtggtgcattaagtcagattacagcttcttcattattgggagattttgggaataacataagaaaattatcattaaaaatgatagatagtaatttagcacattttattgcatcagatgcacatagtataacaaatcgaccttttatgctaaaacagttgtttaatgatagaaagttaaaagcttattatgaggaattagaaagttatttaaaaaacgggaagttagttttaacaaatgaaagaatttccaaacagataccaactcaagattataagcagaaaaaatggtttggtcttttatagaaaaagtgagtgaggggattaaaagtgacaagtatttctgcgaaattgcgatttttaatattaattattatagattcgtttattgttacattttcagtatttttaggatatgcaatattagaaccgtattttaaaggatattcaatagatttattagtattatcatctgttatattgttggtatcacatcatatattcgcatatgtatttaatttatatcatcgagcgtgggagtatgcgagtgtaagtgaattgatgtcagttttaaaagcggttacaagttcaatagtggtaacacttttattagtttcattacttataagtgaaagtccatttctaaggttatattttataacgtggatgatgcatttgctattgattggtggctctagattgttttggcgggtttatagaaggtattttattgataacgctgtcgaaaaaaaagccacattagtagtaggggctggacaaggcggatctgttttaattcgtgaaatgttaagaagccaagatatgcgtatgcaaccagttttagctgttgatgacgataaaaacaaacaaaaaatgactattactgaacgtgttaaagttcaaggttatgttgaagatataccggaactagtaaaaaaatttagaataaaaaagattattattgctataccgacattaagccaaaaaaggttaaatgaaattaataaaatatgtaatattgagggcgttgaattatttaaaatgcctaatatagaagatgtgttatctggagaattagaagtcaataatttgaaaaaagtagaagtagaagatttacttggaagagatccagttgaactagacatggcattaatttcaagagaattaacgaataaaacaatattggtaactggagctggaggatcaataggttcagaaatttgcagacaagtaagtaaatttgatcctcaaaaaattattttgttaggacatggagagaacagtatttattcaattcatcaagaattaagtaaaacttatggaaatagaattgaatttgttcctgttatagctgatgtacaaaataaaacacgtattttagaagtcatgaatgaatttaagccatatgctgtttatcatgctgcagcacataagcatgtaccattaatggaatacaaccctcatgaagctattagaaataatattttaggtactaaaaatgtagctgagtctgcaaaagaaggagaagtaagtaaatttgtaatgatttcaacagataaagctgtaaatccatctaatgtaatgggagcaacaaaacgcattgctgaaatggtaatacaaagtttaaatgaagataattctaagacaagttttgtagcagtaagatttggaaatgtgcttggatcaagaggatcggtcatacctctatttaaaaatcaaattgaatcaggtggtccggttacagtaactcaccctgaaatgacacgatatttcatgactataccagaagcatcaagacttgtattacaggccggggcattggcacaaggtggcgaagtatttgtgctagacatgggtaaaccagttaaaatagttgatttagctaagaatttaatacggttgagcggtaaaaaagaagaagatattggtattgaattttcagggattagaccaggagaaaaattatatgaagaattattaaataaaaatgaaattcatccgcagcaggtatatgaaaaaatttatagaggcaaagttgaccactatattaaaacggaagttgatttgattgtggaagatttaataaataatttctcaaaagagaagctcttaaagatagcaaatagataaatataaaatgtatattggagttttaaaatgaaactaaagtacaaagtaattttaattataaattttgttacagtattatttagtatatttacattcatcggttatttaaataacttgataggttttagagttgttacaatatcattatgtataactatagctatgaccgtgtacttactttacaaaaaaagaaaaggttttttattagtttatttaatatatttatttttaactaattttggtgtatttgtaacaaatatatttttagccaatcctttagtggagtatcacggtgatttatcttggtattatataaatacatctaatttgtttagcattgctacttttgcgatattaacttttacaattttaagtaattttattagtgtgtttagcaaaataaatccaagtagaaagtttgatattaaaagtaaaggaaataatttattctactacacaggaatattatttatcattggatttactatacaatttcttttttatattataactggtcgattagcaattaatacttatggagattatgtaagtagcatacaagaattaccaatgtatacgtacggtatattctttttttcaattgggattgcatttgcattttctaatgtaaaaaaaacacatattaaatatctagtaattatattaactccccaagtgttattttttttaataaccggaaataggggagaagtattttatccaattctatctgcacttggagtgttaatagtaaggaattataaaattaaatggtggatgataattacaattgtttttacattattttttgtaataccatttattaaagtctttagaaatatggatagtagctcaattgaaaaggttgatataaattggttttcttcacttgttgaaataggctacacactgcgccctttaggttatgtaactaggtggattgacggtggggaaagcatagtttatggcaaaagttacttagcacctattcaaaatatattttcatatattataccagggttacaacctgtaaactatgaaatggttggttacggttttaggtatagactacctggtatgggttttaatgtaatagcagaagcgtattacaatggtgcaattgttggagtattgattgtaatggtgttattagtgcttttactttggaaatttacaaattttaaatcttttgaaatgctgtcaatgggtactgcaattgtaagtgtattaatcaataatataagaaacgcattttcattcgtacctgcatacatattaattatcattgtaatagtgattatattgctgtttatagatagttatttaaaaaaagaaacgaaggccgattaaagtggttaaaaattttaattatatgtttgttgctaatatactgtctgcattgtgcaaatttttaattttactagtaatagttagattaggaacacctgaggatgtaggacgttataattatgctttagttataactgctccaatttttttatttatatccctgaaaataaggtctgtaattgtcacgaatgataaatatagtccaaatgaatatatatcagcaattttatcattaaatattattactttaatatttgttgcaatttttgtttatgtattaggaaatggtgatttaactactatattaatagtatcgttaataaaattatttgaaaatataaaagaagtaccttatggaatatatcaaaaaaatgagagtctcaaattactcggaatctcaatgggtatttataatatactaagtttaattttgttttatattatatattctttttcacacaacttgaatatggcacttttatttttagtaatatcctgtatattctcatttgctattatcgatagatggtatctaagtaagtattataatataaaactacactataataacaacattgcaaagtttaaggagatttttattcttacaatacctcttgctttttcaagtgcgttaggatcattaaatacaggaattcctagaatagtattagaaaatctatttggaaaatatacattaggtatattttctacaattgcgtatgtactggtaatcggtgggttattcgcaaattcaatcagtcaagtttttttacccaaattaagaaaattatataaagatgaaaaaaaaattgaatttgaaaagttaactagaaaaatggtgtttattggaatttttattggtatgtgctcggtaatattgagtttgtttttaggtgaggccttgttatcattgttatttggtaaagaatatggtgaaaataatataatattaatcattctttcttttggtttgctttttatactcagtggtatttttttggggacaactataatagccactggaaagtataatgtgaattacaaaatttctctaatactattgttttgtattttgatatttagtttcttattaataccaaaatattctttattaggtgctgctttaactattactatttcacaattcgttgctttaataagctattactatttttacaaaaggatattttgaggtggaaaatatgaaaaaattatttttaaagttaatgaaaaggaatctatctgaagataagataaggaaattgggtgtacaagttggaaatgattgtaggtttttaagtgttgatagatcaacatttggatctgagccttacctgattcaaataggaaatcatgtaacaataactagtggtgtaaaatttgctacccatgatggtggggtatggatttttagaaaaaaatatcctgagatagataattttcatagaatatttatcggtaataatgttttcatagggattaattcaataattttgccaggagtaacaataggaaataatgttgtagtaggtgctgggagtgtggtaacgaaagatgtacctgataatgtaattattggtggtaatcctgccaaaaaaattaaaagtatagaggcctatgaaactaaaatattagaaaatgcggattacacaaaaaaacttaattataatgaaaagaagatatatttattaaataaattcaaagagaataggtataattaatgattaaagttatgcatatatttagcagaatgaatcgtggcggtgcagaattaagaactatggatacaatgaaactattaaatagggagtttgagtttcatgtatgtgctacatcaggaaaaaggggcgaattagatgatgaattagaatctatgggtattacgatacattatttagatattaaaaaatttagttttcctttcaagttcataaagttattgaaaaagaaaaatatagatgttgtacatagtcacatactttttatgagtggattgattcaattactttctttttcagcaaatgtaagaaatagaataacacactttagaacttccaaagatagtaaagaacaatataataaaataagaaaagctagaaataaagtactgaaagccattatagaaatctttagtacgaaaatcttatatgtaagtaatatagcaaataggaatttaatctcaatgaaactttttccgaaaaaacataaaactatttacaatggatttgaaataagtaatataaacaaaaattttaaaaaagaggaaaatagttttatttatgtcggtaggttcattcatactaagaaccaattatttttgttagatgtaatagaaattttaaaaaaagaatttaatactaacattgaaattacttttgttggcaatattcaaactgattatggaaaaaaattcttgagtatagcaaatgaaagaggtttaaataaaaatattaaggttattggagaagtgaataacccattagattatttaaaaacgagtgaatattttttgtttcctagtgaattagagggattaccgggagcattgatagaagcgcatcatcacaattgtattgttatatcttcgaatataaaagaaaattctgaagtaaatcaatattttaaagactcttcatttgaattagaattaattccaaaaacttgggcctcaactatcaaaaagctaatttcaaggaaaaagcatataagttttaatgattctaatgtttttgatatcaatatgactacacaagaattaaaagaaatttatatgagtaaaactttatagatgaagggattgattttcatgaaaatattaatcacaggtacagcaggttttattgggtcacatcttgcgaaaaaactaattaaacaaggtcattacgttataggtgtagatagcataaatgattattattcagtttctttgaaagaggatagattaaaatcaatagggaaagagaatttcacttttaataaagtgaaattagaaaattatgacgatttatctaaagtatttgtagatgaacaaccagaagtagtagttaatttagctgctcaagcaggtgttagatacagtattgagaatccaaggacgtatattgattctaatattgtcggttttatgaacattttagaatgcagtaggcattttaatattcaaaatttaatttatgcttcatctagttctgtttatggtgcaaatacttctaaaccatttagcacttcggataatattgatcatccgttaagtttatatgcagcaactaaaaaatcaaatgagctaatggcacatacgtatagtcatttatacaatttacctacaacaggattgagatttttcacagtatatggtccatgggggagaccagatatggcgctatttaaatttacaaaagcaatcgtaaatgatcaagctatagacgtctataatcacggaaatatgatgagagactttacttacgtggatgatattgttgaagcaattagtaggcttgttaagaaaccagcgtcccctaataaagaatggtcaggggccgaccctgaccctggttcctcatatgcaccatataaagtctacaatattgggaacaacagtccagttagattgatggaatttgtagaggctatagagaataaattaggcaaagaagctaggaaaaactacatggatttacaaccaggagatgtacctgaaacttatgcaaatgtagatgacttgtttagagatattgatttcaaaccagaaactacaattcaagatggtgtaaataaatttgttgattggtacttagaatattataaaaaatgaagaaaaagggtgttttaatatgcaattactctttgtacatgatttcccagttgaaaaatataaagataattattactcaataggcttctctcacaaaatttggaatcgatatttaaccatttttgataaaatgctcataaattcaagagtgaaaaatgtagataatggggaaattattaataaatcgaatggagaaaaagtgaatttcaaaacaatagatagctataaatcacctaaatctctaatttttaaacataaaaaaatatttgaagcccttacaatttctatcaagaaaagtgatggtgttttaattagggtaccaagtgttttaggatttattgcagctctaatatgtaaaaaaatcaataaaccttatatggtggaagtagttggtgcagcatttgacgcatattggtttcatggatcaatatttggaaaaattttatcgttacctatggaatatttacaaaaaaatgcagtgaaaaatgctagcatagcgatttatgtaactaagaaatatttgagtaacaaatatccttgtaacgggaaagagtttaaaggcatttcaaacgtacaatctgtagaaaaatttaataagaatttagatattgggaataaaattaaaattggacttattggatctacgtttgttgattataaaggacataacgtagctataaaatcaataagtaatttggtgaatgaaggttataacattgaattagaatttgtgggtgatgggcccagtaagaaatttatggaaatggctaaaaaatacaatgttgaaaataatgttatatttaaaggtaaaatttatgacaagacagcgttaaataattggtttagaaatttagatttgtatattcaaccaagtttaacagagggacattgtcgagcaattgtggaagcaataggaaatggagtgcctacattagcgtctaatgctggtggtaattcggatagtgttaacaaagaatatttgtttaaaccaaaagatgtagttaaattaacaaaactaattaatagatctattttgtctaagcaatatcgagaagaaaatgttttagaaaacaaaaaaaatatttcaggttacaacctggaaaatattcaaatagaaagagaaaaagcactacttaattataaaaaaataataaatgatttttatttagcgaaaggaataaataaaaatgcttaattatatatataatcattcaccaataattttccaaaatttaatggtttcaataaagggtaaaatattcatgaaacaaagatatactaaacattattatgaagaaataaagagacttagagagtgtaatgatttatttgaacttcaaaatcaaagatttgaagaattttataattatatcaaaaaaaatagtgaattttattctgaaataattaaaaaaaataatctaagcggtaaaaaaatcactgttgcaaatataaaccaattgcctgaaattacgaaagatgatattagaaaaaatgttgataaaattattactaaaaagaaaaataaacttataaaaatgggcactggcggttcgactggtaaaagtatggttttttatactaatgcatatgatatgtctagaaaaatagcttaccttgattattttaaagaacaacatggggtatataaaggtatgaaaagagttagtgtaggtggtagaaaaattgtaccaattaaacaaaaaaagaaagtattttggcgatataataaaccactaaatcaattaatgatatctgcttatcatgctgatggtgaaaatcttaaatactacattaaaaaattaaataaatttcagcctgaaactttggatggctatactacagttattcataggattgcgagatatattttagataacaatatagaattaagttttacaccaattgctatttttcctaatgcagaaactttaactgatttaatgagggatgatattgaaaaagcttttaattgtccagtgcgtaatcaatatgcttcttctgaaggagcaccttttattacagaaaacaaagaaggcgaactagaaattaatgtagctactggagtgtttgagtgtaaacaaattcatggtaatatctatgaattaatagtgacaggtttttatactactactacaccattattgaggtataaaatcggagattcagtagaattagaaaatgagcttcccgtaaattatcaacaaaaagatataaaaattaaaagaattatcggtcgtaacaatgattttttacagtctagagaaaaaggaattgtaactaatgtgaatttgtcaacagcaataagatttgttgagaatgatgttattgaatcgcaatttgtacagaatgatattgataacattattgtgtatttagtaattagtaatgatgcggataaaaataatattataaagaaattaaaatatgagttgaaattcaggtttggtacaaatactaatttccattttgaatttgttaataagataccatcaacacctggtggaaaaaaaagatttgcaattaataacattaaataaggggataagaaaatgaatagaaatattgcagtagtaggtttaggttatgtaggtttaccagtagctgtaacttttgggaataaacataaagtaatcggatttgatattaatgaatcaagaattaaagaattaaaaaataattatgatagaacaaatgaagtaacagaaaataaattaaaaaacacgaatatagaatatacttcaaatgcagaagatttgaaaaaggctgattttattattatagctgtgccaacaccaatcgataagcataataaacctgatttattaccattattaaaagccagtgaaactgttgggaaggtaattactccagacacaatcgttgtatatgaatctacagtttatcctggcgcaacagaagaagaatgtgtacctgtattggaaaaatattctggactcgtttgtggtaaagatttttttgttggttactcacctgaaagaattaatcctggggataaggtacatacttttgaaaccattactaaggttgtatctggccaaacgcttgaagtattagaaatagttgcagacgtatatagttcagtagtcacagcaggagttcataaagcatcttctattaaagtagcagaagcagcaaaagtcattgaaaacacacaacgtgatgtaaatattgccctaatgaatgaattggcaattatttttgataaattagatatagatactaacgaggtattaaaagcttcaggaacaaaatggaacttcttgaattttaaaccaggattagtagggggacattgcattggtgtggacccatattatttaacacataaagctcaggaagttgggcaccatcctgaagtgattttagcaggtagaagaataaatgataatatggctaaatatattgcttctaacgttattaaagagttattgaagcaaggattagaagtacaaggagcaacagttaatgtgctaggtcttacatttaaagagaattgtccggatttaagaaatacaaaggttattcatattattgaagaactgaaagagtatggattaaacgtaacagtgaatgatgttgaagcggataaaaatgaagctaaaaagttctttggtttagatttgatagatacaaaagaattaaaaatggtggatgtagtgttatttgcagtgccacataaagactatatggaaaataaaaaggattatatcaatttagttaaagattgtggcatagtgtttgacattaaaggcataatcaatagtgatgaacttaatgtaagtcaacgattatggagattataagtgtaaaattacattgtgaggttttgtatgaaaaatcaaaaaatatttcatttagttactgtttctaagagtattccacttatgagaggacaaatagaatttttaagaaaaaaaaatatggatgttcacattgtttcgagtgatggtaaggaattaaagcagtatgataatgaaatagctcatgttatacctatgaaaagagatatagcattattcagtgatttaaagtcattattaaaaatgatattactatttcacaaagaaaaaccatttattgttaattctggtactccaaaagcaggattaataggaacaatagctgcgtttattacccaaagacctattagaatatatactgtgagaggtttaaggcttgaaacagttaaaggattcaaatattttgtattgtatttgatggaaaagatagcaatgttttgtgcaactgatataatagcaatttctgaaagtttaaagcataaaattattacatctaatttggctaaggaaaataaaattactgttttgggatttggtagttctaatggtatacaatttgaaaaattccaattagataacaataaattagaagaaaaataccataaattattaaatgataattttgttattggctatgtaggaagaattgtaaaagataaaggtatacatgaattaattcagtcatttaaaattattgtaagtaaaggatataatgtcaaattgcttgttattggtagtttagagacagaaaattctattgatgaatctgactatttatttttaactcaaaatcctaatgtagtactaatcaagcatgtttcagatccaatttcattttataataatatgaatgtatttgtttttccaactcatagagaagggtttggaaatgttagtatagaggctcaagcacttgaagtgccagtaattactactaatgttacaggcgctattgatactgtagtaaatggagaaactggatttattgttgaaaaaggtgactttaaagcaatcgctgaaaaaattgaaaaattaattaatgacgagagtttaagagaaactattggtcataatggaagaaagagagtggaaaataaattttcaagccaaattatatgggaagaattggaaagtatgtacaatacttttctaaaagaaagtgagggaaagtaatgaaaagaatatttgatatatttagttcattatatgcaattattatatttttgccgctgctctttctagtgtctattgcaattaaaatagagtctaaaggtccgattgtatttaaacaggaccgaccaggcgtaaagaacaaattatttaaaatttataaatttagatctatgagacaagataccccaaatgtagcaactgataggttagaccctgcagattatattacaaaaactgggaagttcattagaaaaacatcattagatgaattaccacagttatttaatattttaaaaggtgatatggcagtagttgggcctagacctgctttatataatcagtatgaacttattgaaaaaagaactaatgtaaatgttcatttggttaagccaggattaactggcttagcacaagttatgggcagagataacaatacggacgaccaaaaagttcaatatgataagttttatgttgaaaaccaatctttcaaattagatatgtatattatttataaaaccataaaaaacactatatcttctgaaggggttagtcactaatgaaaaatatattaatcacagggaaaaatggatttgtaggtaatcaatttcaattattattaaataatgataattataaagtcgatagagtcagtttgaaaaataatgattggaagttttgttcatttgaaaattatgacgtaatcattcatttagcagcattggttcataataatcaacccaatgctaaaataattgattatatgaatacgaattaccatttaactagagaactagctaaaaaagctaaagatgacggtgtaagtcaatttatattttttagtactatgggtatttttggcatgaatggtttaattaataaaaaatgtgaaatcactcaattaactccatataaacctaaaagtgcatacgactattctaaattgttagcagaaagagatattcaaaaattaattgatgacaaatttgtggtaaatatcgttagaccgccaatgatatatggtaaaaagggaccgggtaatttcgctaaacttataaaagttgcaaatttatcaagaatatttcccaaatatcataatgaaagaagtgttatacatatagataacttatataaacatattttacacttattgaagaatgaaaaatctgatatcacacatccacagaatatggaatatatgaacactaatactgctttatcattgattagaaatcatttaggaaaatcgagtgaacttatagaaattccagtgccaaatttcattaataaaattattgaaaaatcaaatattgcgaacaaaatatatggaaatttaacatactcaaaaaatatagacgatagagaaataaataatatgcattttgacaattttaatcaaactattagtaaaactttaaaataaaaaattatatgataaaagatttgttatgataaataaatcgttaacgaaaaaataggcttaactgttataattataccaatattattattatttatagtaggttttgtttttataaattaatagttttaaatagcttaattaatgatctactagaaaaggcaggttcccatttctattgactattttcgattaatgtgaaaggtcaaaaaatagatgtgatcagtgctttacctgagtacacacactaaaattgtcggtagaggtacataagaaaagattaatcatgtgtatgtgtgttgtgaatcgtacatggcagtaaaatctattgaaaaattaattaatgtgccaattggcattcattatgcaacgattaatttagatggtgatgaacattactttatggagtgaatggtgtcgatgttgtgagtaacagtgatttttatccccaattatcccttctctaaaggtcaaaaaataccacatggatggtaaagaatcttcagcgtttatacgtaatagaaaagaagcgggtgctggtagtggaggaaatagacaagcgagacaacagcacgttattgaagcagttactaaaaaactatgaatccaacctctatcatgagaaccaattcaattaataaaggggcaaaacacgttaggtgttggtcgacttcattacttctatcctggtgataatgaagatattattcaagcatatcgagacaatttgaatttaaataaaattttagtaaacatcaattgctcatttagcggttggtgttttttatagattaaaatatatggaaacacagatgtaaataacctttttattaaagttatacttacagcatttgagttttggaggagttggtattaaatgaaactacaggcagttttgatttatgatatctaattgaattggcacatataatttattattataattcgagtataaaagttaggaatgaagaaaagctattttagccatttatacatagctatgttgtaaatatttagcaaaataactaatatggggaggcgtatcataagtaggggggatgttgataagcttccccttgttagttatttggctaaaaaagactagctgttcattatatttacatggtatgtcaacattttttagacagttttcatgtgtgtatttttaaatgccactgacgtcaaatactttaatgaagaatgtggtctagaattgttgtaccaatttacataatcaaataattctgtttctaactgttctaagttttcaaattgcatttgttttacaaattctgttttcattgctttcatcgttgcttccgcaactgcgttatcataaggacaatctttggtacttcatgaacgtttcattttaaatgtttctagaacttcatctatcaaatgattattaaattctttgcctctatcggtgtgaaatagtttgatttattctgctgattgcttttgatactatatttgcgtccttatttttaactgcactgtaaccaacaatttttctattaaatagatctataaataaacatatgtagtgccatgttcctgcgacttttacgtatgttaaatcacttactaatgcttccattggttgctctctattaaaagcttcattcaaatgatttttaatttgtcttgtaccaagacctttctattagaattaaaaatatttatgattgcacgttctacgtttgaatcatctttagtgattttattatcttttctttttatagaatcataataggtacttcttggtatttttaggactttacatattgctggtactgaatattgatgtgcattcttttgaatgacttctattttcgtctcataatcagctctgcttgctttaaaatatcgttctccatttttatatgttgaacttctttgcgtaatttaatcagctctttttcttcatctgataaattatcttgatgattgaacaaacctgtgtattgatgttgctttatccattttcctaacacctaggaatgaaacctaaccatttgtaacttaaactctgaactaaacgattttctttctcttgtcataataaaatcacctactttcttaaattaacaatatctactctcatagaatttgtccaattaagtgtagacgattcaaaatatcaaatttatatgtatagcacttcttgctttttatgattaacttagcaaaaaatacaaatcaacaataaataaaatttaattacttaatatatatttattaccattagagtctgtttacagcatcctttttgatttgttcatcagatttttctttccatgcatgaggtgaaaagttaaatgtaatactatggtcatcaaaaaactcataaagggcaggtcttaacctttttgctttttcatttcttctttctgatctttttcgaccctctgataaacgtgtaacacctgtacaataaattttatccatttctttgatatcatctttatatagttcctttgataaagaaatattttttctttacttgaattaacatttacttcgttaggaattgcttcattaagactctcataaaaaggaaatagccctttaccaaaactagatgattcgatgccaaaaaaattatatagcatactgtaatcatctactattaatccattattatttgcttcaactaccaaggaatttttcatagttgactctttataaaatgttaattgaactttcgcgaatttatttattcttttaataaattttcctttaaatctgcggataaagactaaataattttcatttttatatttaaatttaaatgaatcaataatataatccttatcacgcatatcgtcaaatattaattgaattttagacaagtgtaaattgttcatagttatctactccctatatataagtggatttctacataattattctcctaggaaccctaaaatcggcattatatctttcaaaaatgtacctcataaaaagaataggttgatatcgactttaatttactctctttctttattactaaatacactttaacacatttgtaaaatcataatcattcactctaacatagtaaaattcataattttttcttaagtatattaattgtatattttcatattaaagtccaatgaatatactttattcatttattagtcgaatttttcatatttcattaaccaactacctacttaaattatttaatcttattcttactcatcatttcaaagtgttggatatgtctaagtttttcaaagactaacttaaagactcttttataatttctcatacttgctccatcaaaggtaataacaaaatcatcacttataaaatgtgatccgtcatttattcctgaaataagtgatttacataatattttgtctataccgtcaaatttattaatacatctttcataatccatgcccctataatattaaaataatattatagtattctacgcattgtattaaatgatgttattggtgacatttcttcagaattaagttctttccacattaattgatatgaagattttatagcattattctcataagtattaaaataacttatattatccttttttctaataattccatacattacttcattaagcgaaaacctttctctacttcctaaaaaagtgatttctttatgaaagtaaatattgtgagtaagattgaatacttgttgtattctattcttattatttctacagtcatttatcaaatttttaactaaagtactaacaataaataatacattactatctaaactagaaattggatcatctataatagcgattttatttgaagttacacttgtactctcatgactaccataaataaaatttatttttttaggttttatttctacataatttctataggtcgcaatgttttgaagcgaaatttttttaatcattcataataccccaatcccttattttaattagtaaagacactatatttatcaactgctaactgtgggctttagatttatttctgataatcaaaattatgtctgagatgttactaatgtatttaatattatttttagtatttaatctcccccatacttatgatacgcctctgcttatcagttaataatggctctatgtcaaattggactgataagttcaatattggaagttaggcaactaaacattgcttaacttcctttttactttttggagcgtaaagttttgaacataataatattcgattgcgtaaatgattgtaacttccataaccaaaagatatacgtttaattaattttattttgttatttataccttctaaaggaccatttgataaattgtaataatcaaatgtattttttataaagtatgcatgtttgtttagtgtcttaattgctcgttggacaaatgaatgcgcttgagataatggcatatgttttaattcgtgaataaagccttcataatcattatgtttaagtttgaatcgtaattgattgatatattggtaggcgtcattcagtgagtcatcttgatctaatatatacttgacgatacctttttgtgtcttccattccttaaataaagcgactttacgatattcaaatgcctctaaatcttcttcaggttttaataataatttcgcatatcttttaaacttattatataaaggttgattcgtcgttcttaattcattcataacagcgatgtcaatagcggtttaaaatactgttttatggcggtttgaaaatatcgttttttagcgggttaagaatatcgtgcttttaatgcgcttttacttttatttattctcatgtttacgttcttcatagcaatttttagtttagataaatctgaaagagtagcagcttcaaaatttgcatttatatctaaaaataattttaaaatagacatgtttaaacaatcaaaagtgtgcattgctaaatcatcattttcaaaagtttatggatagaacaattatatgtaaacatatttatttaagttttaaaaaagtaggacttaattatcgcaatatttggtctatataaatgaaatatgatttaagtgaagaaataatactttgatatctacagaattaataggagtgatcagtcgtataacagtaattgcaatttgtgaaagttaattttgtaattgtatcaacaatttttaaataagattattaggtaagtcataaatagccctgttaagggataaatgatataatagtgctatataggaggcgatatcgtgattactattgactcaaagattaacaaacaaatcgctaaaacctttcacttgaaggtatgtatgttacaaaagaacaacaaatacaaattcttcatgcaattaataataagaaattacaaatgagcttattcgtaaaatcgcttttaaataatgaaatatgatggaatacagcaagacgaatatttattaaattctaatatgcttggtgtcaaaacttctgatgaattaaagcgacttggaaaagttgcttttttaattgctgaagcttccgttgaagaggaaggatataaattctatttcccttatcgtttaaagtgaatttcaagataattatcaaaaaagcatatatactatattaaaagtggacgtagggacagtcattaaaatataaaaggattattaaagtgcaatttgttgtaatataataattaaattttaaaaattatagttacattaagtgaatatatgttataaaataacaacggttgataagggtcaactaaattaattatatattcaagaggagtaatatgagaaaagtatttatactaatcactttgttatttggttatagtagctattctttgttggaagctaaagcagagacacaaaatgatccaaatataagtgaattaaacaaatctagtcaatatacgggttcgtggcataatatatggtatttatataatagtgatccagttaatgctaaaaaaataaagctaagtgacaaatttttgagtcatgaattcattgttccaataaataaccctagtcattacgactatgttaaaactgagttaaaagacagtacaatggctagttcatttgatgggaaagaagtcgatatttttggcgtcaattattttgatcaatgctattttttaaatgaaaatatacagtgtgatagtaatcaaggagctggaagcaaaaaaacttgtatgtatggtggtataacattaaatgagaacaataccaataatagaattcagcctattgttgtaaaagtttacgagaatgacagtgttactctttcttttgatatcaatattgataaagagactgtaactattcaagagttagattataaagtgagaaataaattaatttctaaaatcaatttataccatttaggtggcacttcatatgaaacgggatacattaaatttatagaaaatggtaatcgatattattggtatgatatgatgccagaccctggctttactcagtctaaatatttgatgatatatcggggtaatgaaacagttgaatcagccaaaacggaaatagaagtgcacttaactaaaaagtagacatagagcaacttatcaataataatttatggctagattaaagtaaataaaaatattttgatgaattaaagcgacttggaaaagttgctttttttaattacttctttaaaaagtaggtgcattagtctttaatacaaacaaaaaaggaggaacattacattcctcctcaacctttattactcatactataattcaattttaacgtcttcgtccatttgggcttcaaattcatctagaagtgctcttgcttctgcaattgattgtgtgttcatcaattgatggcgaagttcgctagcacctcttatgccacgcacatagattttaaagaatctacgcaaactcttgaattgtcgtatttcatctttttcatatttgttaaacaatgataatgcaatctcaacagatctaatagttccttgcttgtgtgttcacgtggttctttttcaaaagcgaatggattgtggaaaatgcctctaccaatcatgacgccatcaatgccatatttttctgcaagttcaagtcctgtttttctatcgggaatatcaccgttaattgttaacaatgtatttggtgcaatttcgtcacgtaaatttttaatagcttcgattaattcccaatgtgcatctactttactcatttctttacgtgtacgaagatgaatagataaatttgcaatgtcttgttcaaagacatgctttaaccaatctttccattcatcgatttcatagtagccgaggcgtgttttaacacttaccggaagcccacctgctttagttgcttgaataattttggcagcgacatcaggtcttaagattaagccggaacccttaccctttttagcaacatttgctacaggacatcccatatttaagtcaatacctttaaagcccattttagctaattgaatactcgtttcacggaactgttctggcttatctccccatatgtgagcgaccatcggctgttcatcttcactaaaagttaagcgtccgcgcacactatgtatgccttcagggtggcaacagctttcagtatttgtaaattcagtgaaaaacacatccggtctagctgcttcacttacaacgtgtcgaaagacgatatctgtaacgtcttccattggcgccaaaataaaaaatggacgtggtaattcactccaaaaattttccttcataatatatttataccctctttataattagtatctcgattttttatgcatgatgatattaccacaaaagacgaacttatacaaaaggaattttagctgatacaaccatttgaaagggaagtctacgagtagtctaaaatgaatattgtggttagttgatcagtatacaaatcaaggattatcgtattagattgttcattattaatgatacactacttattaatatgattcagaattttctttagctacatatacaataccgtgatttttacgttatcttataacaaagacaaatttataaaggtgatattatggaagatttaaaacactctttaaaaggtttaggttggtatgacttattttttacagtacctatgtttctactattcgtgtatctgccgaattataatttcataactatatttattaacattgttattattattttcttttccataggtttgattttaactactcatataattataggtaacattaagaacaactctaaatgatttattaatacaaatatggttaaacataaaactgaaggagcgattacaatggcgactgagaaagatgtaaatgatttatttttaaatcatgtgaattcaaatgccgttaaaactagaaagatgatgggagaatatattatttattatgatggtgtggttataggtggtttgtatgataatagattattggtcaaggcgactaaaagtgcacgtcatcaatttcaagataatacattagtatcgacgtatcctggtgccaaggaaatggttttaattccacactttgtcaaagtaacaactctaaatgatttatttgagctcataaaaaatgatttgcaaaagtgaagtgcgaatagagagagaaataaaagcggataatatgtccgtcttcgccacccttaaaatatattattgtaccttttatgaggtaagtagctaaaggttgttcgaactaagcactagtacttcattttctatttcaattctttcttaatttgattaggagatttcctctcctttaaaggtcgttgcttttagtaaatttctttttgctttgtatgttcgaggtatatcattattaaaaaatgcctacaattaatgaggactatagcaaatgtagtaccgataatgattagaatgtttttttgatatgatatcaactccgatattggtaaggacataggagcaattacaatttccaaacaaggtacacgagtgccattgaatattttattcagatcaaaagtacacgcactaaaataagcatttatttctatgtaacttacatctttgatactacagaagttcatgcatatgttcaatcgtctcataatttttaaatacccctgattcccatagttaaatacattattcaaatcgtaattaaaacatagaagtatttgaatcaccatattcaacttttaagtttgtactactatgaagtaatctaactaccactttagtcaatacaaatataacggaaagataatcaagtgagtgctcattcaggggtgctcttttttcatgtatttattgaacaaaaataaagatgcttaaggcccatttaaaaagctacggaagtcatttgagaaataatatagttacagtaaaaagttatacataatatcttcatcagtgaaagccctttaaaatatagaactttgatttaagtaagaaaaatttaattcatgtgatatttatgaatgtttagattaaagatgatatatactattaataacagtattacttgaaaattaactaccttgtatatttgaatataataatatgatgtgactgtttttatacaatctaatttaaaactaaactgccaatattgaatattatggggtatagacaagttatatgtagccactttttgagagtactttattataatagtcttgtagtcaatttgcagtgacaagagctaaaaaagaattttatgtaattggggacatgcaaagaatacagacaaaaccattttatgaaaccatttttgaagaaagaaatgtagaataacatacaaaaaaagtatatagagggagttattttcatgcaaaatagaaatgattttatagaaaaactattagacagagctcaaattgaagaaggtatgcgtgtactggatataggttgtgcgactggtgaggtcactcaattaattgcaaaacgtgtgggggcgaatggtgaagtcgtcggtgttgatgtgaatgaatcattacttaaaattgcaaatgaaaacaatcaatataacaatgtatcatatcaatattctgatatatatcacttgccagacactatggggcattttgatgcgattgttggtagaagagtattgatgtacttaccagatgctgaaaagtgtttacaaattttgaatcaattttaaaatctgagggtatattatgttttcaagaaagtgatgctattaatacaggcgttggtgcagatgcactacctttacatcaatcggcaattcaatggatttgggaaacagtgaagcaagaaggtggcaatattcatattggtcaaaatttatataatttgtttaataataatggaatgtatgttgtagattactttgcagaggcagtgattcaaacgtcaatggataatgatttagcatggcttgtagatgtaatgctaccaagaatgaaagcgcatggtgtcattaatgatgatttttcactagatgagtttaaatcaaaccttgaacatgaagcaataaacaatcaatgtgcatttatacgtgaaatggcgtttggtattatcggaaaagcgtagtttaaaaaacgctgaaagtagcatattattatcaacaatgttttaatttgtgaagttattaatggaatttgaaatgacagcatttctataggctaatcaagtgatacttaattaccgagcaatttatacattttaaaaggagtaaaattgaataatgaagaaatttaacaactggatattaaatgcaataagtggatctcaaacagacaagaatggaacaactgaagaattaaaaggggcaaaatttatcattttatatgcatattcaatgctcgttttgcttgcgttagtaatttctaacatattcattcacattttggagcctaaattatcaatcaccactcaaatcatcatcgttttgattttaattgaatcactaattggactacgtttcttgcaaagggtacgatgttaggcgtggcaaagataaagaaaataagaaaaatagtaaggatttcgttaaactaaaatcaattttagtagcaattttatttacatcattggtgctgacagcaggcactgtagctgatatatacggtttcactgacttaggaaatactagaagtgattaatcgtttggagcataggtggtattatatttggcctcgtaagttacacaatggaagataaaaaataacgataaggagttggcgattataaagctagctccttttttaacttatttatgtaaagaactatcccgcgggtgtttaatcatatgtcaataattcctataatacattgttaaaacatcaattaaataagttttaaaattttacccatatttttatttgaaaaagatgtataattaatgtattgaatatagaaagaatttgatattatgaaaaagtgtattaagactttgtttttaagtatcattttagtagtgatgagtggtttatatcattcagcacatgcgtcagattcgttgagtaaaagtccggaaaattggatgagtaaacttgatgaaagcaaacatttaactgagattaatatgccaggctcacatgatagtggctcattcactttaaaggatccagtaaaatcggtttgggcaaagactcaagataaagattaccttacgcaaatgaagtcaggagtcaggttttttgatattagaggtagagcaagtgctgataatataatttcggttcatcacggcatggtttatttgcatcatgaattaggaaaatttctcgatgatgctaaatattatttgagtgcctatccaaatgaaacaattgtgatgtctatgaaaaaggactatgatagtgaccctaaagttatgaagacatttgaagaggtttttagagaatattattataataacccgcaatatcagaatcttttttacacaggaagtaatgcgaaccctactttaaaagagacgaaaggtaaaattgttctattcaatagaatggggggcacatacataaagagtggttatggtgctgacacgtcaggtattcaatgggcagacaatgcgacatttgaaacgaaaattaataatggtaacttacatttacaggtacaagatgagtataaagattattacgataagaaagtagaagctgttaaaaatttattggctaaagcaaaaacggatagtaacaaagacaatgtatatgtgaatttcttgagtgtggcgtctggaggcagcgcatttaatagtacttataactatgcatcacatataaatcctgaaattgcaaaaacgattaaagcaaatgggaaagctagaacgggttggctgattgttgactatgcaggatatccgtggcctggatatgatggtatcgtaagcaaaattatagatagtaataaataaggattaaataatgacattaagacgagtatgaaaatagttagattctaattattttcactactcgtttttattttgaaaataagtaataattcaacaatattataaattgaacagattttttgtgaaatttttgataatattaaagtgaaaaagtgttataaattgataaatatatgtaattcgaaaaagtgaatcattttaaaaagaagagagtgatgaaatgataaaacgtgtaaataaattagtgcttggtattagtcttctgtttttagtcattagtatcactgctggttgtgatatgggtaaagaagaggaaataaagaaaagttttgaaaaaacattaagtatgtatccgattaaaaacctagaggatttataagataaagaaggttatcgtgatgatcaatttgataaaaatgataaaggtacatggattattggttctgaaatggcaactcaaaataagggtgaagctctgaaagttaaaggtatggtcttatatatgaatagaaatacaagaagtgcaaaaggattttactatgttaatgcagtaaagaaagatgaagatggtagacctcaggataataaaatagaatatccggttaaaatggtcgataataaaatcatccctactaaagatattaaagatggaaacataaaaaaagaaatcgaaaactttaagttcttcgcgcaatatggtacctttaaagatttgtcgaagtacaaagatggagatatttcatataatccagaggtaccgagttattcagctaaatatcaattaactaatgatgattataatgtaaaacaattacgaaaaagatatgatataccaacaaataaagcgccaaagctgttgttgaaaggttcgggagacttaaaaggttcatcagttggatataaagacattgaatttacttttgtggagaaaaaaggggaaaatactttttttactgatagtctacatcttgaaccaagtgaggataaataatcatgaccaataaagagtatgaaatcgatcccggaaaaagagagtgatgaaatgataaaacgtgtaaataaattagtgcttggtattagtcttctgtttttagtcatcagtatcgctgctggttgtggcattggtaaagaagcggaaattaagaaaagttttgagaagacattgagtatgtatccgattaaaaatctagaagatttatacgataaagaaggttatcgtgatgacgaatttgataaaaatgataaaggaacgtggactattagttctgaaatggcaattcagaaaaagggagaagcattaaatataaaaggtatggttttgaagttaaatagaaatacaaggagtgcaaaaggattttattatgttaatgcgataaagaaggatgaagatggaagacctcaggataatcaaatagaatatcctgttaaaatggtcgataataaaatcattccaacaaaagaaattaaagatgacaacataaaaaaagaaatcgaaaactttaagtttttagttcaatacggtaactttaaagatttgtcgaagtacaaagatggcgatatatcatacaatccagaggtgccgagttattcagctaaatatcaattaaccaatgatgattataatgtgaaacaattacgaaaaagatataatataccaacgaataaagcaccaaagttattgttgaaaggtacaggtaatttaaaaggctcatcagttggatataaagatattgaatttactttcgtggagaaaaaaggtgaaaatatatactttagtgatagtttacatcttaaaccgagtgaggataaataacctcattgtctcccacacgattcaacacgaatttcaaatatgttgctgtaaaataatgtaaaataaacgtattcatattactgaattgagggatagtatgcaacgtgattatttaattcgagtagaaactgagagtatgtcagatttcaaaaggctcaatggtttaatgattggttttgttattaaaggtgaggcacatatttatgatgaaaataatatgacgcaatgcaacagtggcgacattttcatcattaaccaccgcgacttgtatcgatttcaacttcaacaagatggcatcatatgttatatccaattccaaatgaaatatttagcagacaagtttgatgatgcgcattgtctatattttcacttaacagatgcgaccacaacaaagaataaacatcaactgagaaatataatggcaagactggtttcaacacatatccgacataatgagttgtctaaattgactgagcaacaacttgtgattcagttgcttatgcatatgattcattatgtcccgcgtacatatcattcgaaccaaagtatcttaaatgatgataaagtgaatcaagtatgcgactatattgagttacattttcatgaagatttaagcctttcggaattaagcgaatatgttgggtggtcagagagccatctgtctaaaaagtttgcagaatcactaggtgtaggattccagcatttcttaaatacgacgcgaattgagcatgcgaaactcgatttgacatatacagatgaaatgattactgatattgcattgcaaaatggcttttcaagtgcagcgagctttgcgagaacatttaaacacattacgcatcaaacacctaaacagtatcgaggtgatcgtccagcagtcactgaaaaccagcaatcggcacaacatgattatcacgaccgtgaattgatattacttttaaatgactacattgaagaaatgaatcatttcattgaagatattgaaatgatgaactataaagagattacctttcaaccaaccaatcaacaactaaatcaatttaatcatatcattcaagtgggctatttgaggaatttgctcaatacacagtatcaatcacagttgcttacatgtcatcatgattttcaagtcaatgaagtattagcatatgatgtgatgccatatatcatgaaaaagctcaatgcgccattcacgtatgatgcagaaatttcgaatatattttatgatattgatttgtgtttagactttttattagatcataattttagtctgaccatgcatttggattagtatgactcacgagattatatcgatgcattcaaagtatttatccatcacgttgccctgcatgtcagtcatagaaaagatttgaagttcaacttgtatgtgacgacattgcacacgtctttgattgaaatgattgattattttaaagcattattccctaatggtggcttgtacattcacttagatcaagctacggaaaggcagctaccattgttgaaacgacttgagccacacatcgaccattttgtatttgatgccaattcaaataatgctgttgattttaataaaatgaatgatgatgaatttaaaactgcaagtcaaatgattattaataaaacgaattaccttatcgatttaatgcatcgtcatcatctaaagcgtccactcattttactcaattggaatacattgacgggtgatacatttattacaaacggcgaatgttttagaggtggtatcatcattgagcaattattaaaattaagttctaaagtagagggtatcgggtattggttgaattatgacttgcacgtcagtcattgtagaaatgaacgggattatatgaattctattgaactgtttcatcaatataatggaaaacgtccggtctatttcacggcattgctatttaataaattaacaagcaatattttatattctgatgatacatgtattgtcacgggaactgattcaaattttcaaatattgttatatgatgcaaagcattttaatccgtacttagcgttggacaatcaaatgaatatgcgtgcaacggaaatgatccatttgaacattaatgccctggaagaaggtatgtataagattaaacattttaccttagataaagaaaatggtgcattatttaatctttggcgcaaacatcatacgatacatggtatggacaaggactctatagattacgttaatcgaatgagttttccaaaattagaagtgtacgatatagatatgacggatacgctggcattaaatattaaaatgattacaaatggaattcacttaattgaagtaaaacgttacccaagttcataaaatgatcacaaatcacaaattttgatatacagaatttgtgattttttatattcaaagttaaaattgcaaaaaattaatggttaacatctctgttgttggcaatataaataatgattaatcatttatgatgtaactaaggagatgaatgatatgaatcaacaattaattgaagctttaaaatcaaaagaagacaaaatgattgagatcagacgttatttacatcagcatccagaattatcttttcatgaaggtgaaacggcgaaatacatcgttgaattttacaaagataaagatgtagaagtagaaacgaatgccggaccacgtggaattaaagtaacgattgattcagggagacctggtaaaacattagcaatccgtgcagactttgatgcattacccattactgaagatacaggattgtcttttgcatcacaaaataaaggtgttatgcacgcatgtggtcacgatgcacatacagcatatatgcttgtattagcagagacgcttgctgaaatgaaagatagttttacaggaaaggtcgttgtgatacatcaaccagctgaagaagtaccaccaggtggtgctaaagcaatgattgaaaatggtgtattagacggtgttgatcatgtattaggcgtacacgtcatgagcacaatgaaaacaggtaatgtgtattacagacctggttatgttcaaacaggacgcgcattcttcaaattgaaagttcaaggtaaaggtggtcatggttcatcaccacatatggccaatgatgccattgttgcaggtagctacttcgtcacagcgttacaaacagttgtatctagacgactaagtccatttgaaaccggcgttgtcacaatcggttcatttgacggtaaaggtcaattcaatgtcattaaagatgttgttgaaattgaaggtgatgtacgtggattaacagatgctacaaaagcaacaattgaaaaagaaattaaacgtttatcaaaaggattagaagcattgtatggtgtaacttgcacattagaatataacgatgattatcctgcattatataatgatccagagtttactgagtacgtggctaagacgctaaaagaagcagacctggaatttggtgtcgaaatatgtgaaccacaaccaccttcagaagactttgcatattatgctaaggaacaaccaagtgcctttatttatacaggtgcagctgtggaagatggtgaaatttacccacatcatcatcctaaatttaccatttcagaaaaatcattacttatttcggaagaagctgtagggacagttgttttagattaccttaaaggagataactaacatgaatgaaacgtatcgcgggggcaacaagttaatcttaggtattgtattaggtgttattacattttggttgtttgcacaatcacttgtaaatgttgtaccaaatttacaacgaagttttggtgcagatattggaacaattagtattgcggtaagtctaaccgcactattttcaggcatgtttgttgttggaacaggcggcctagcagataaaattggacgcgtgaaaatgacgaatatcggcttattattaagtattattggttcagcattaattattattacaaatttaccagcattgctaattttaggtcgtattatacaaggcgtatcagcagcgtgtattatgccttccacattggccattatgaaaacttattatgagggtgctgaacgtcagcgtgccttaagctactggtctatcgtttcttggggtggaagtggtatctgttcactcttcggtggggcagttgcgacaactatgggttggagatggattttcatcttctcaattatcgttgccgtactttcaatgttactcatcaaagggacgcctgaaacgaaatcagaagttaccaatacacataaatttgacgttgcaggtctaattgttctagtagttatgttgctaagtttaaacgttgtcattactaaaggtgcagcacttggttatacatcattatggttctttggtttgattgcaatcgtaattgtagcattctttattttcttaaatgttgagaaaaaataggtacatcatcgttaagaccaacaagtgcttcagaaacatttcgtgaatgataaccgatacgttcaagaacaccaatcatatcgatatatagtaatccgccttttgttgtacattcaccacgattaaggcgtttaatatgacctttgcgtagtttatgttcaatattaaatgattctctactacgctctacaatttcatcttttttcgttttgtcataaacatctaacatgtcgatggctttatcaaatgactcagcaacatggttgaataatttatccataccgcgttgtgcatcttctgtaatgcgaatatcttcatcatgttggcgttttaattgagcgacatactcttctgttagctctgctacttttaaaatagagcgattgacatcaaacataactgctaaacgctcaacgtctgccttcgtaatggcttttgtagaaattctaactaaataatttcgaatactatcattgattgtttcaacagcttggtgcttttgttcaagctttttgatcaattttttatcgtcttttgtaatttcgcgaatgtcttcaaacattgataagacaatctgacccacattttgtaattctttttgagtttcttgtaatgcaacaccaggtgcgtgataaacaagatctttgtttaagtgctgaggtttatagtcatcagcaatatctttacctgggacaagctttgtaactatccatgctaaacctgctacaaatggtaattgaatcaaagtatttgttatgttgaagataccatgtgatactgcaatcgtcatcgctggttttaagtgccatacatcttgtaacaaactaatcaaatgaatcacaactggcaagaaaattgtgaagataattaccccgattaagttaaagatgacgtgtacaagcgccgcacgttttgcagcgattgagccggctaaactagctaagatagctgtaatcgtggtacc

SEQ ID NO: 88 - DNA, 57474 bp, Staphylococcus epidermidis
  source 1..57474: mol_type = genomic DNA
cgaagcatatcataaatgatgcggtttttttagccgcttcatgaagaaaattagagttagcacaatacttttgttaaaatataaagacaaatatttggaaatataaaaataattaataattaataaattctcttatttttccatttgaatctctatataaatttttttgcaaaaggacatcttcttcagattcaatatctacactatctaccaattttttatttctaacatcagtaagtcgtgtataaaaaaaatatataaaaggaattaataaaaagaatcttaattttttcatagaaattaccttccttattcaatagctataaacttaaaataacatttattttaatattagtaaatatatttttgaatattaaataacaatcgttaagaatgtataacacttataaatatattattaaaagtagatgtatataatgaaaaattatcaaaaagtagtaaaattaaaatttacctttcacagcacatttgttaaaattattcatgatatgtaaatttgatgtaaagattaaattataatatatgcatatgagaaaactatgctctttagtattaggtgaacattttagggaggaaaatagtttgcgatattcaatagttatgccttataaagaaacagatacaaagatgtttaatgattgtttagatagtcttaataatcaaacttttaaagattttgaggtattatttatacatgatggagattcaaatttgcatgacgaaataaaaaatattaattttaatcataaagttttagaatctaatgtttcatcaaatcctcaatattatagaaatataggtattaaggaagctacgggtgaatatatcttgtttatggatagtgatgattacttacaccctaattcgttaatttatgccaatgaaatcataaatgaagaacacgattttgtggttaagctaggagtaaaaaaaactcactataccaaaagtttaactttcaaggaaaacactcgaagtttttataaacctgaaatttccgaaaaaataagaaagctacttaccgatataaataaagaaaatgtatctaatccagagtttattaataaactatatgagcttgaattagtaaagcatagttataataatattaaacaaaataaatttttagacaatattaattatcgtttaaaatctaatgggttaataattaatagacaattcataatagatcatgaactaggatttgatacaactaacagtttatatggagatattccgtttgttatcaaattatataatttagtagatagaataaaacagacaagagtaaaattatattttaaattaatacataacgatagtattagttatccgtcacatacacaaaaatttaaagaaactaaaagctattataaattattagcgtatgataaagcattgaattattgtcctaatttaaacttagcgagaaaattaaaaggttttgctacaagagaatatttatattatgtatgtaaaggtgaagattttcgaaactcatttaacaatgtaaaaccaatatattcaattttacgagatttactaaataaaccttcaaaaagactcaaaatcaatttgcgtcataaattggaaattaatcctattaagaacggggaatacagaaaagcctataataggtcaagaagaagagttaagttgtataatgcttatgaatttttgaaacctaaaaatgaaagatatagaaaaaaagttatacaaaaaaatttttttgcaaaattgccaatacgtaaagatttgatcgtttatgaaagttttttagggaaaaattactctgatagccctaaatcaatttttgaatatcttaaatcatctgagagcgataaatttaaacatgtttggattttgaataataaagatattttagaagattatccaatattaaatgataaaaatgtgaaaattatcgatagatttagctgggaatatttttattatgtaactgtcgcaaagtattttatactaaatatgagacagcccaaatggttagttaaaaagaaagatcaaactattttatctacatggcatggcactcctttaaagagattagtatttgatatggataatgtaacttcagcaagtaaaagttataaacaagatttttatcagcaatcaagaaattgggattacctaattgctgctaataaatatagtgagcaaattttcgagagagcattcaaatatcctacttcaaatattttaacttatggttatcctcgaaatgatatcttaagtaattataatcaagaatataaaattaaggttaaacaaaaattaaatatacctacagataaaaaagtcgttctttatgctcctacttggagagatgatgaatatcatggagtaggtcaatacaaattttcattaaatttagacttagaacaaatgagacaagaattgggggaagaatatatcgtactacttagaatgcattactttatatcagatagattagatatttcagaatatgaaggatttgcgtttgatttttctaaatataatgatgtaaatgacctttatattgtaagcgacatattgatcacagattattcttcagttttctttgattatgctaatctgaagagacctattttattctatacttatgacttagataaatataaagatgaactgcgtggtttctatatagatatggaaaaagatttaccaggaccgcttttatttgattcatcggaagttattaataatataaaaaatattgaggttataaataataattataaagagagatacgaattattttataataaattctgtttattagatgatggaaaagcaacaaaaagagtcgttgaaaaggttattaactaatatttttaaatgaaaaaataaattaatagagcatcccttaccgcaaagtgaaggatgctctatttttgttgaaattaatataagcttcattctgatagatagtcattaataattgtttgcaaatcgttattaagtaccgttgttacagcgtcatcatccataaaatcctcgtagattttatcaagaatttattcctcttacagacattcgcgagaagtccgttttaattttattagaagtaattcaggtttgaacctacctaaatgaatatatgagttatcttttatactacaaaatatattcagatttcaataatgacataaaataggcatctttatatttacctttagtgtagaattgctctttgagtaatccttctgttttaaatccttgtgactcgtatatatgaatagcttttttgttatctgcatcaacatatagatagatcttgtgcatatttaaaatattgaatgcataaattatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctttccgattcatctaataaatgcttatcaaataaatgttgaagttccgttaaggattcataaggctcttcaaaccaataagacataatagaatattcattatttaattcatgaacaaaaagtaaatcactatattctaatgctcttagtttcataattccactcccaaaatcttcatatatatttgcattataaatataaataacgaataagtcatcatcactgtgaacactttttttaacaattcatcacatattaattctcattttcttattattctcgatttattactcttactatgaaatctataaaattctcatatttgtttatattaagaataaatacgtcaatagtaacaataaaaaataaataatagagcatccctcaccgctaagtgaaggatgctctagttttattgaaatatacatttcattttgttaaataattattaataatattttgaaaatcattattacgtgaaatcttcatagattttatcaagtatttctttgtcttcaattgctgtgaagtgatgtaccaatccttttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgttgaatgttaaaaatcctataaatgtttattcatctgcaggattttttaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctacatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataaataataattaataatatcttggaaatcattgttaagtattgttgtaatacaatcgtcattcataaaatcttcatagattttatcaagtatttcttcattttcaatcgctgcgaaatgatgtactaatccttttacaattgatacataactttgcgatgctaaacaactagcaactgtatgccaatctgattgtttagtttcatggttcatagataatcatcctttatttcaatgtccatttttgacgtgctttaggattaagcggatgcataatatcatttgttgctggatttatcaactttttcaccttctttttatgaggttttaacatttccattacctgtgatacacgttcaacgacagttggccgcttcgcataagctccataggctagaatcactgtgtcactttcacttatcgctttcatcaagtgaatatcagtgtgctcatcatatggatttttgatgtgtttgagattttctggacttttaatattagagaatagatttacaagatatacagcgccatattgttcagaattagctaattggttgaggataagaacagttgtgagatcgagtgataatacaccgtctaaatgaggatacatcgttatcactgtgcatgcagctttcttttcatcccatgttttcttgagtaaatagcggtgctgttcatcatcgctaaatatggcttctgtgtgtatcgtacttttgattgtattcatcatcgtcacttcctttagtattcttctggtaaaagcatcacataataaaaagcgtccacgtcatcttcacgaatgacgtagactttcttaggtaatgcattttgatttttttcatagtttgtatagtgatattccaatttgtatgtgggttgttcttgttcatgcgtaactgacaacatattatcatcttcttgtagtttgaaaatgtgtaggtaatcggtatgagagtggttatctctttcttttaccatattccaaagtatgatttgaaggtctagagatagttgttcactaatgcctcttgtgatgtatcgattgattttcatactatttgcctccattttgcttttctttcatgatgtcaatcacttcgttaatgactgtaacagatatttgtgccactttgatccaattattcatggtcctgacctccttgttttagtaaatgacgttcatcaataatgatattttgagtatctgtaaggtacagaaagtccatatcaaaatgatccagataaccaatactgatgagttggttattggcatacattagaaatggatagatacttagctcatgtagctcatcattatagtaggtataagtctcgagtgtgagttgtaccagtggagaatcattaataaaacgttccggtagaatattttctgctgcttcctccagcgcttcacattcccaagcttcgttattagatagttggaataagtgggtcatatattgtttgagttcttgagtgatggttttcatattattgcctcctagatagtgtaatagtgatgtagttcatatacatcattgagataatatatatttgatttgtcatttattacgaatcccggtgggaataagagaaaattccatatgaaaaaccgcttcaaaccttggtatgacaaggaaatcccgaaattccgcctgttttgacgaacaatcaactcattatttataagtattgatgatagtggcgggtctttacttccttatatatattatttatttataaagaataacgggattttgggattgtgcttgcacaatccttctgcttcttcgaatctgcaaatcccattcctttcccgataaaaaatcatcgtgggatgttctttagcaatttcaatataagcttcgtgtagtagtagatagatactgtcaattcgctctctcattagataagtgttattgaaattgataaagagaattctcaaaaatttttagaaaaagaaagaggaagtatgtagatataaaatagatttattaaatagtaagatataatgtttgcggtaaaaataaagacgaagtgctggaatacacttcgtcgataataaggtaattgaagtctcgattattttaaattgatcattttaagcctacaaattcctctaatgtttcaccagatttgtacacttttgtcgccttcttttttcctaaataacgtaaatgccaaggttcatattgatatcctgtgatgctttctttgtttttaggatatcttataataaatccataattatgggcattcttagctatccagcgtccttcctcagttttaccaaaactagcatatagatttttatctgaatcgacgccaccaacatcaaatgtaagacctgtttgatgttcagaatagcctggacgcgcactatatttatctgctgcttttttaccatcacgcttcacataattattatatagcttaacttgtgtaggatagcttctatatccacttattttatataagtgaatatcatctttttgtgcaccatcaaataattgttttaaggctttttgtgcttttggattttcacccggattataagaggagggtaaatcaatatttttgtttacaattaaaattccatctgcatacgtccgaccattcttaacaactttttgatgcttcttttggttgttatcgttattttgggcttgatttgtatgtgcagttttactttcatttttgtcattgtggctttcagattgatgtgaacaagctgaaagtaataaagcgctagtcatcaatattacagataattttttcatattaatactccttttgctaatacaaaaatattaaatgctaaatataacataacatatactttgagctctatatataatatttaatattttataatgattcaattaatgcgtatataatttatattatgttaccattaaatatacatgatacgagttgatttaaagtaagtcgttcatttttaatatcaaagtcacaaatataaaatagaaattgaggaatatgaattgaaaaaaatatttttgattatgttgatatctttattttgttgtagttttttcacaaatactacatatgctttgactcctgttgaaatcactaataattcaaaagcacaacttgatgaaggattgaaccctagaggagcagtcgtgacaacaacgaatggacaaatattatataagtatcataaggataagaaagttgatcctgcatcaacaacaaagttgatgacaatgctagttatatatgacgacattaatcatagtaaagtgagcttaaaagacaaggttaaaatatctgagagatatcaaaaaatgtcacaattacctaatttaactacctttccattaaaaaaagggcaaacatatacgattgaacagttactaaaacaagctgccttaaattctagtaatgcagcaacgctagtattagcagaacatatagatggagatatttcaaaattcactgatagaatgaaccgtgaagcgcaattattaggtatgaatcaaacgcactttactaatcccagtggtgccaacaataaaattataaaaccctatgaacccaaaaaatataaggatgaaactagctcatatacgacagcaaatgatatggctattttaacaaatcacttattacgcaagtatcctaatattttaaaaatgactcaattggaaacagatactcaatacaatcaacaattacataacacgaatttatcactgcctcatcaatcactcggcatgaaaaatgtggatggattgaaaacggggacaagcaaagaaggatataatttagcattaaccgctaaaaaagatcaattaagagtgaatacaaatttatttaatatccagccatatcctagtgaaaaagctaaatttgcacgccacaaggttgctaacgcactcacccaaaacgcatttaaaaattatacgtatcgcaaagtgatatcgaaaggtgcacatcaaattgatggtaaaacttacaacgtgaaagaagacttatatgatgtagtacctaaggataacagtaaatatgaattaaaaatatcggaaaaaaaccaactatcagttaaatacaatcgacaatttactaaaggggagcatattcctagcgttaaagtagaaccaaagtttaacttcttgtctgtattatttcaaatcactttagcaattgtcggtataatattggtttctgtcattgttattatagcgatcaaagtgtacataaaaaaatatagtaaaaattaaattaatttctttttaagaaatagaacatgtgtaacaaatgtataaattaaagtagatgcaactttttgtgttaatattctctatttttatataatttatatatttaaaataattatattattcatatagtactaaagattaaagtattatattttgaataaaggcgaagtgctacaaagcacttcgccataatatccagcatttttattaattaatacttgtgtatcaatgtagatgtgcgctagtttcttcaacttctcctctagctgttttttcgtacatttctagttgcttctcataatatgatttatatcatattaaggtgtgagacctcagccagtctgtgacatgacatttttttcatattgggcttctttttgaggggagtcaaattttccaacgtaatcgatttcgtttttggttttgtgtataacttctccactttcattcgttcttatgtcatttattattacgttattatttgcattaggttgagcaattgtattattttttgaatttagattatcaccgctgtcatttcattcaacaggctcttcttttttatctttatcattttgcttgttctttatttttggtagttttatgagcttgagagtcttgtttttttattgttcatatctataacagtcttgtaactaaatactaacgatacaataattacacttattaaagcgactgtagataatataggggaaaaatatttcttataaacctctctcaatataatttaatcctatattgcattaatttaaaaaacataccactgaaattatattgttaaataaaaaggttggggtaaaaataaagacgaagtgctaaggagcacttcgtcgagaggaatatcattgaaaacctgtttaattacttcattatcaagtttgagtgttacataaaactgttttttgtgattgctgtcttttctaatatctatgcgatcaatcactgctaagtacagtgctttaagttgagattttttcattttctcaatattatgaaagacgtatcgcaatattttaccaatttttttggcattatatgagggtttatcttcattttgttgttttttgagttgattgatttgactcataatgtcattaagttgcttatcatatttttgaatcgacggcctgattactgatgttaagtccggattatcctcgatggttttaatcaagttatttagtttgattcttacttcatcatattgttgttgcttataagcgatatcgtgatgaagtgacgcacaatcaacttgattttcttgattaacacgtgctacaacgcgttgaatgactttatcacttttgactatttcgagtatttgcttcatcacataatcttcaatcacatcagctcttacactgtttgccgaacatactttggaccccttgttgcgaaaattactacatgaatagtaacgtatacgtttcttggttccatctttaagtgtattcgttgtattgcttgctgccataggtgcgccacattggggacagtgaataatgcctgtaagcagatttgttcctttgccatggacttggggtttttgactgacttgttttttacgtatttgtactttattccataaatcttgattaataatgggggaatgcttcccttcagctatcactggtttatcattaagccctttacgacgtttttcactccaatctttgtatttcgcaaattgaattttgccgatatagaatgggttagctaatatatatgtgatagaactaatactaaaaggtttaccctttttagtgacatagcctttgtgattcaatgcattggctattttacgatagccatgtcctttggcataggactcgaatatatattttacaatattagcttcatgttggttaatcattagctcgtgtttactatctggtattttgtcataacctagtggtaaattgccttgataatagccttcttgggcacgtctcgtttgccccataaatacattctcgacaatgttattacgttcgaattctgagaaactcgcaagtatttgtaacatgagtttaccagaagaagtattgacttccatacgctctgataaactgaaaaattcgacattttgtttatgcaaatcttcgacgattttcagaagatccgatgtattacgagctaatcggtttgttttgtagatcataacacaatctatattgccttcttttgcatctttcaacatacgttggagctcaggtctgttcataaatttacctgaaataccacgatcagcgtatatatctttgacctcaaaatgatggaagtcacagtattctttgatttgattgatttgtccatcgatactataaccttctgtgctttgcatttctgttgaaacacgtacatagataccgacacgttttgttttaagttgttgcattatgttacatcctttcttcatttatgcaatcgatgattgcatggcttgattgacgatattgagtggttcatttttgaaatagattcctataagatttttatctttcgtaatgtgaatggtttcaatataggggtacaatatgtttaacgtgaaacgtttttgaataatattttgaatggtgtgttgtatttgatgcccatttatagatgtagtgcgttgcggttgttgacgtaatgattgcgtttgttctctgaacgtttctgcatcaatgatgccttgtgccaacttttctatcagttgttcttgagtcaatatgtgatgtttttctatgtttctttgtcttttgatgcgtttgtcaatcgcacctttaatttttgtgtagatgcgttgattttgataaaagtctcggcacacttctaatactttatcttcaagtgtttgtgcattgatgcctttaaaatcacagacaaagcgtgaagcattcatgtttttaggacagacgtagtaacgtaatgtatgattcttttttctaatggtcatatttgtaagtgttgcattacaacatgggcatttgattttttgtttgagttgattatccgaagatgtctgtttggtttgtttttgcgatcgaagtctctgcgcttgctcatatatacttgtggaaacaatagaaggaaacatattgtcgaattgaccatattgattgttgacacgaccacaataattaggattgatgataatgttacgaacttgatagggttgtcgattgatatacgtgttatcttcttctaataactgtgcaattttcttataaccatgacctttaatgtaataattgaatacagcctttaccgttggtgactcattttgattgatgatgaatgttccgttgtgataatcgtaaccaaaaggtgcatgtgttgtaatcaatcgaccttgctttgctttttcttgaagcccatttctgacttgttctccaatgttatccgattcaagttcggctaaactgatgaagatattaagcttgagtcggtcgaaagcttgatccatatcaaagtaaccatcatgtacgcttaagatatgaacatggtacgtttgacataatttgatgagttttaatgcatttttcagattacgatgcaacctattaagacgataacagcataatatgtcacattgtccttgttgaattaattgtgttacttgtcgatacccactacgattatctttgcgtcctgattgtttgtcgctataaaagttgatatgttgaatatgatgtttttcggctattgcttcgatagcttgtttctgtgctgcaagagattgttgtttcatcgtactttgacgtaaatagcctataacttgtttcatatcggctcctcctttcacagtgataatatatatttatagatgaattgatatataagcccaacatcaatgagatgttgggcgtccatattagtcattcgtttgattgatttcttcaattaccaaatcggctaatatctcgataagttcatccatgtttttcactccgttatttgttctatcttcaatacgtcgattattcagtttgatgcttcacggttgtatgataaagacaatcagaaatcttcgtgaactcctgaagggcctatcccctcattagcggatttaaaaagttctttcgcagctttgttatcatttgccggtgtccaattttgaattaacgacttatctttagttaatcccaggatagatgcaaactctacatctaattttagatggtaaaatacaagtgattgttttttaccgctattatctttgacacttcttttagttgtttggcgtccacggtcagctaatatgaaacctttatctcttaaggcgttgacaacattattaacatcttgaaattgatgattgtttagcatctgtttaaaaacgtttgcaatcatttttacttcgatatggtcatcttttaatgagattaatccatagttctcaaacatatttttcaaagcaccttcatctgaaaacttacctctgttttgtgctacaaattgaatgatgacatcgatagctttatcagctaatgagcgttcagagattgtatgaccatgataatcaataaagtagtctcttattttagcgatatcaatatctgtagctaaaacacgacctaatatttttgcagatgttgtaatgactgcataacgcttaaacatacgattgcctgtgttgcttttatcatctttcaatttagcttcaaaccaatctacttccttgtaaaaccattgaataacttcatcttcacgatttataagatatttagctactaacggtaaaacatgaccatagtttagtgctacagcttttttaatattgtcagcattggtcgcatttgtagtgaattgttcattaatctcgatggttcttacacgtaatccatcgttttgagctgaatcattaaaaatactgtgttctgacgttgaaatgacagaagtaccccaattcttaggcgttttaacttctccatgaacgtttgaacgttgacgaccttgaccttcagtgatggagtacaataaccccgttgtatctctaaaagttgctgatgagagttcatcaaatacaataggtataccaaaattgttactcaagtaaccttcaagtgcattacgtgtggcattccaatttctaaagagagtttcattacctttggtagggttaccagcgactgatacagctaaagaagctgcagttgacttaccggttgaggattgacctgtaaaactaaaaatgattccggcaaattcggtttcatgtttgtgcttcaggaaactcgtcactaaggcagaaataccaaatacgactgctaattctaaaagaagagaacctttaacctcttttagatacatgttaaaccaattatcaaatgtacctttaggagttaagtcataagcattatcacaaatggcgtcagatggagatttattatcaaattctttagtagtatagatttcacttaacgatacaataggaccaaacggtgtttccagtatacctaccccttcatataagtaggaaatgggtaattgattgcgcatttgttgcaacgcataacctaaatcttttgtatatttttcattaatactaaatccatacttcattaaagatggcagtttttgtgtcgttaaaatatcactagattcaacaatttctttttgatcttcgtctgtgataattagtttttcagtattagttttagggtcaataaacttattttcgataacgataggacctgcgatttcaacttcagtaggcattcctcctttttcttttgggggcgtgtctttataccaacctttttttgatttgtatcgtggtgaaggattaaatgaaaggttagtttgagccattagcaatcacctcctttcgaagggttgctgttatggtgtggattaggacctgttttaagataaactaagtgaccgtgagtatccttaccgataataataaatggaacacgtggcgcatgttttacaaaatatgcgaaccaacgtccaacattttgttgtacagcttttgaacattgtacatttgcacgactgttcaagtcatgaaatgtaaattcttctccttcaggtaaattgaatgaaatacctaatattttcttttttaactgctcgaatttatctgattgattgaaatacatttaacttcctcctactaatgaactaagataggaaaattaaatatgcgcacaattaactttcttaagttcattttagccaagaagttatcatttgaaagcttgtaaaaatatgttagaaattccgtactaaattaagatggatttttgttgacaaaataaaaaacgctgatttaacagcgctttaaataaaaattaatctgaagttatataaaagtagtcagaaggagtggtgttattatcattataataatctagtgctttattgatttcttttattaatttatcatgatgttcttttgttattttctttggatatttgagttctttttcttctaagtcttttggagacactaggtcactatttaaaattaatgataacagcatatttttaggtgccgaagctctggtgtctcgatgagtaatatattttgctaaattactgtttatacttactttttcattttctaaaaagtttgtactatttttcacatgagcatcagcaattttggtacttatttcagtgtttaaatcgtctatttcatctaaataatattcaagaatctgaagtttatctttagaagtgatacctttttcagtgaaaatcttaataatgatagatttgattccaatttctatattactgtctactagaaaatagagataataattagtcagagcatgttcataatctctttgatcagtaatttcatgattgatgaaataatataagttctctttgatacgattagaagaatctatatctttgtcattgataccgtttttaaaaaagtgtagataaactgagttgtttgagagtatatcatataatacaaataaactttttggtttaaatacatatgattttcctgatttaaagtttgctatgtttattcctagagcgttgcagaatttattaaaattagttgtaaaattatttttttcttgaataaacatgtcgttgtcatttaattctgaactatgatttttaatcatattgatattttcttcaatcttgtcactatttaaattttccatgcaaagtatactatatcttactatatcacttatatttatggaaaaatctttaggagaacgatttaaagtatcctttttattatcttgatctacagccctacatgaatcatcaaaaacttctttgacatttattttatcaatgttcaaaattatgtctactaatgcattttgaaatttttcattatatatgtctctcttattcataatatcactacctttatcaattataataatatattatatcttaatatttgtagaagtacttgatgagaggttagataaaacaccacatttctctggttagtaaaaacataacttctaagactttttttcatatactaataaattatattataaatatttttgaaaataaaagtagtgtataaaaatgtatgcaaagaaggtgttttagtatagattttaaggcattcatttttaatataaaatatttataatattttatattaaaaatgattatcctttcaaaatttttgcaacgagtaattttactttagttatattataaagtagaattaatagtaattaaaggaacgtttgttcctgtttttatatgtaaatacgagaacgattgaatagataaagaaggataaaattgacttcgaaatagaatatttaatttcaagaaggagagtattggatgttaaagcataaaataataaatttaatacaagaaaagagagaagggagttactgggattttaaagcagaatatcataaagataaagcagaattattacatgatataatttgtttatcaaataacttgttgaatcaagaagcttatttaattttaggagtcgcagataacggtcatattctgggggttgcaggtgattcaaatcgaaaaaaccaagaagaattaatatcatttattactggaaaaaaatttgctgcgggtaggcaccccaagatttcattaatgacatttgaatatgaagaaaaggaaatcgatgtcattataataaatcctaaaggatatgtaccatactatctggagagagcagaaaccgatcaaaaaagtaaaaaaaacaaaacagtcaatgcaggtagcatttatacaagagttgaagataaaaataccccaatagattctactgcaagtccattagatacagaaatattatggaaaatgcattttggtttatatcctactcctataaagaggttacagaattatttactaactcctgagaaatggatgcagaactcaacaggttattttcatagtgaatcgccagagtatatagtatacaaaaatgaggacattgaagaaaaagaaaattattttaatttagtaagtccattttatgcgtataatcaaatcaatagtaacactttatactcatattacgaatttaaatatcatagtacagttttatatggttgccggtgtatctctttagattcaggtatctacacaacaccagtacctgaactaggtgagataaattttaatatgcatagagatgatactatttattatcgttatttcattgaagagacgatgctttataatattcatctttttatgtataagggtgattcgatggaagagaaatttgcaatggataaatttttagaatgtgttttggtttataaaagtgatgtagaaaaggaactttttgaaaattatatattagataattgggataaagttaatcagtcaattaatgaaaataataaacgtgtatttggaactgaacatttgtcacaacttgaaaaagaagatatcactaaaaaagtaaaaacagttaaagttttaaaagatgaacttgagaactttagaacttgagaaaaattattggttaataggagaaaatcatgaaaaaatcaacactcattgataaatttttagacttactttcttcaagaagctcactcagagaaattcagaataattttattgatgcaaacattatgagagatagttctataaatcaaaaatataacggacaacgtaaatcgttagcgtgggaatatattagtactttaaatttagaagatgaagctgaattttctaaattacttaatgttatagaaacgtatttgtttcaatggaatctgtatatccatgaagttgatgaagacgaagaaattaatagattaataaaaataattaatgtattaggttatgaatataataaagatacaggaaaaataactaaaaatgaaagagaagttaatttaagtactattaaatctttggcaataaaatttgatattgaatatgtattaaaagaatgtaatagaattgaaaaagaggcactaacggatccagaggacgcaattacatctgcaaaatcgatggttgagagtactttgaagcatattttagattctgaaggagaaaaatttaataataatgaaaccttgagaggtttatataaaaaagtaagtaatattatgaatctttctcctggtggacataatgaaaatacttttaaaacaattttgagtggaatgataaatgtaattaacggtctggacgaagtaaggaatgaatatggtgacgcacatggtaaatcaaagaaaaattataggcctgaaactagacatgcttttctagcaatcaacgcagcacgtacgataactgaatttcttttagcttcatataaaaagtaaaagctatttgatgaatagcaatcaaatatattggttataaaaaaccgcatcatcaactgataagcagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgtagaaaactttaattttttatctcctaattacactgaacaacaaaaaataggtaatttcttcagcaaactcgaccgccagattgagttagaagaagagaaacttgaactcttagagcaacaaaagcgtggatatattcagaagattttttctcaagatttaagatttaaagatgaaaatggaaacagttatcctgattggtctattaaaaagattgaagatatttctaaagttaataaagggtttactccaaatacaaaaaatgataaatactgggatgaattaaatgaaaattggttatctatagcaggtatgacacagaaatatttgtataaaggaaataaaggaattactgaaaaaggtgcatcaaagcatgtaaaagtagataaagatactctaataatgagttttaaattgactttaggtaagttagctatagtaaaagagcctatctatacaaatgaagctatatgccatttcgtatggaaagaaagtaatgttaatactgagtatatgtactactatttaaattctataaatataagtacttttggtgcacaggcagttaaaggagtaacattaaataacgatgcaattaatagtattatagtaaagttaccagtgatacaagaacaaaataaaatagcatactttttcaataaattagataaattaattgaaaaacaatcttctaaagtagaattattaaaacaacgcaaacaaggatttttacagaaaatgtttgtttaattcttataaagttctattatgtaaaatattaaatagagataacattatgaaagcgagcccaagacataaagtttttgaataaataaaaaagataatttctatcaaattaatatagaaattgtcttttttataaattttttgattatttttagctgattgagctgttacttttcttataataagtgctattagcacaaatcctagttctcttttggctttgtttattcctcttacggacattcgagtgaaacccattttaattttattagaagtaatttaggtttgaacccacctaaataaatatatgagttatttttttatgctacaaaatatattcagatttcaataatgacataaaataggcatctttatatttacctttagtgtagaattgctctttgagtaatccttctgttttaaatccttgtgactcgtatatatgcacagcttttttgttatctgtatcaacatatagataaattttgtgcatgtttaatatatcgaatgcataatttatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctctctgattcatctaataaatgcttatcaaataaatattgaagttccgttaaggattcatatggttcttcaaaccaataagacataatagaatattcattatttaattcatgaacaaaaagtaaatcactatactctaatgctcttagtttcataattccactcccaaaattttctcatatatttgcattataaatataaataacgaataagtcatcattcactgtgaatactctattttaacaattcaccacatactaattctcattttcttgttattctcgatttattactcttactatgaaacctataaaattctcacatttgtttgtattaagaataaatacgtcgatagtaacaataaaaaaataaataataaagcatccctcaccgtaaaagtgaaggatgctctagttttattgaaatatacatttcattttgttaaataattattaataatattttgaaaatcattattacgtgaaatcttcatagattttatcaagtatttctttgccttcaattgctgtgaagtgatgtaccaatctatttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgtggaatgttaaaaatcctacaaatgtttattcatctgcaggatttttaaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctaaatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataagtaataattaataatatcttggaaatcattgttaagtattgttgtaatacaatcgtcattcataaaatcttcatagattttatcaagaatttcttcatcttcgatagatgtgaaatgattagctaacccttttataatttaagtgtaatttgtgaatctaaacaactagcaactgttcgccaatctgattgtgtagtgtcatggttcatagataatcctccctttattttaatgtccatttttgacgtgctttagggttaagtggatgcataacttcgttcgttactggattaatgagttttttgactttctttttatgaggttttaacatttccatcacttgctcaacacgttcgacgacaacaggccgcttcgcataagctccataggctagaatcactgtgtcactttcactaatcgctttcattaagtgaatgtctgtgtgtttgtcataaggctctttcctacgattagtcaagtacttataattaaaataagcattacttaactaaaccgttcattttaatttttttgaataaggtgttaaactatatttaattgtacgtttggctaatgtggtgacatttgataatcgtacaatttatgattcattacaagataatgaattgtttttaataatcgatttatacaagcaatgatggcagtcttatgaggtttctcattaggctgctttcttagtttgtagtaataatcgacgacatgattgtcataatgatgctgccctcttattatattcataatcacccaaaataaaagttttctcgcttttttattaccacgcttgttgatggtatctctacagtgtgtatgacctgattgatatcgtttgatatcaatgccaacaaaagcattgagttgtttatttgatttaaatcgcttaatatcaccaatctccccaataatcatagctgtgcttagcttaccaataccaggtatcgaatgaatattttcaaaataatcgagttgttgtgctaattgaatcatggcatcatctaattgtttgagatgatgaatagattgttttaattgttgaataagtaagcgtaatttttcgactagaaaggaatgtctatcgacattaggatagctttcttgagcaatcactcttaattgaagtgcatattttgtagctttatccattgacattcccttatctgtagaattgaatatatgtgtaatcagtacatccttgtcgatatcaagaaccatgtctggatgagtaaagatttctgcgatgttgagtgcaatgattgaatatcgactactaaataatctttctaaaccagggaatgtttgatggagtaattcaaggatctgaaatttaagtcgattttgttcattctcgatttctagatgaaaacggacgcgttctcttaattcaaagaatattaactcatgtataggtaagctgtctgtttgtttaagcgtcggtcctaaacaagcaagcttatgagcatctgcctgatcagttttccatgatcttagagcgctcgttttaaatttggcttctaacggattcatttgaatatagttaatttgatttacacaacaaaatcgttccatacctcttgaatagatacctgtagattcaaaaatgagttgtgggtggtctaagtcattcaaatacttgagtaaataattgtaaccatttttattattctggatgaaaaactctttttggaattttccatttttataatgtgcaactacactacttcttttactaatatcaacacctaagtaatcgataaaaaaacctcctttgaataattgagaagctaaaaactttacttaacctttctcatttcattttcctatacacggtttcaagaacccaacatactacaaacgaatttcaaaaggcgagagtaaagctgacttgttttttatacggatttaaaatccaagagtctggacagtctacttctctctataactataaaaaatagctatgaaaaaatctatcgtcatagatttcttcatagctaatcttagtatgtttaatatgtttaaggttttcaggtgttttaatattagagaatagatttactagatatacagcaccgtattgttcagaattagctaattggttgagaatgagaacagtggtaagatcgagtgataatacgccatctaaatgtggatacatcgttatcactgtacaagagggtttcttttcatcccatattttcttgagtaagtagcggtgttgttcatcgtcgctaaatatagcttctgtgtgtatcgtacttttgattgtattcatatatcgttaccccttttaatattcttctggtaaaagcatcacataataaaaagcgtctacgccatcttcacgaatgacgtagactttcttaggtaatgcattttgattttttacatagtttgtatagtgatattccaatttgtatgcaggttgttcttgttcatgtgtgattgagagtatattctcatcttcttgcagtttaaaaatgtgtaggtaatctgtatgaggttgattgtcccgttcttttaccatgttccaaagtaagatttgaaggtctagagataggtgttcactaatgcctcttgtgatatatcgattgatgttcatactattttcctccattttgcttttctttcataatgtcaatcacttcgttaatgactgtaacagatatttgtgccactttgatccaattattcatggtgattccctccttgttttagtaaatgacgttcatcgataagcgtgtttttagtgtctgtgaggtagagaaagtccatatcaaaatgatccaaataaccaatgctgatgagttggtcattggcgtacataagaaatggatagatacttagctcatgtagctcatcattgtagtaggtataagtgttgagtgtgagatgcgcaagtagagaattattaataaaacgttccggtagaatattttctgctgcttcctcaagtgcttcacattcccatgtttcgttgttagatagttggaagagacgagttatatattgtttgagttcttgagtggttgttttcatatcattgcctcctagatagtgtgatagtgatgtagttgatatacatcattgggataatatatatttgatttattatttattacgaatcccggtgggaataagagaaaattccatataaaaacccgtgataaatgttggtgtaacaaggaaatcccggaatcccactcattttgacgaacaatcaactcattatttataagtattgatgatagggttgtgtctctgcttccttatatatattatttatttataaaaaataacgggattttgggattgtgcttgcacaatccttctgcttcttcgaatctgcaaatcccattcctttcccggtaaaaaatcattgtgggatgttctttagcaatttcaatataagcattgtgtagtcatgaaaaaaatgacggcaatgactgtttcattagataagtgttattgaaattgataaagagaattctaaaaatggttagataaaataagtgaaagaatagcagtgtagttattgtttattcaatagttatatataaagtttgtggcaaaaataaagacgaagtgctagggagcacttcgtcgagtggatggttattaaatagttgtttgattatatcattatttaacttgagcgtaacataaaattgcttcttatgatgctcatcttttcttatgtcaatgcgatcgatgattgttagatatagtgatttcaactgagatttctctaatttgtctatatctttgaatattgcttgtagtacattcgcaatcatatcagcatcataatgtggagcttctgcttgtttatcttgttctagttgatagatttgattatttatttgattcaattcatcttggtagctaagtattgttggttttaatacgctatctaagtcaggtgagtcttcaatagttttcgttaatgtatgcatttttgcttttatttcttcacattgtgactgtttataggcaatatcatggttcaaagaagagacgtctatttgacttttttcatttaccttttcaaccaattgcttcaatacctttttgcttttgataatttccaatatctgatccataacatatttttctagtacatctgctctaacgctattggcagaacaaacttttgaacctttatttctaaaattgctacatgagtaatatctgattcttttcttagtgccatcttttaatgtattagttgtattacttgcctccattgctgcaccacatttcggacattttacaatcccagtcagtaggtttgttcctttgccgtgaacttgtggtttcttgcgactctcttgacgtttaaactgtactttatcccataggtttctatcaataataggcgcatgtttaccatcagcgataatcggttcctcattgagtccttttcgtcttttatcgctccagtgtctatacttcgcaaactgtatctttccaatgtaaaaagggtttgagatgatgtaggtaatggacgaaatactaaaaggtttccctttcttagtcacataacctttatgattcaatgcgttcgcaatcttacgataaccatgacctttagcgtatgaatcaaaaatatatttaacaatattcgcttcatgttgattgatcatgagctcttttttactgtcaggtactttatcgtagcctagaggtaaattaccttgataataaccttcaatagcacgttgtctttggccattgtagacattctctacaatcgtattacgttcaaattctgcgaagctggctaaaatttggagcatcaatttacctgttgaactggcaatttctattttttcagttagactaaaaaattcgacattaatcttatacaattcctcgacaatatttaacaaatctgaggtatttctagctaaacgatttgttttgtagaccataatacaatctaatttaccttcgttggcatcttttaacatacgttgtaattctggacgttgcattgttttacctgatataccacgatcggtgtattcattgacgacttcatagccttgaaattgacaatactctgtaagttgattcaattgaccttgaatactgtaaccatccgtttgcatttcagtcgatacacgtgcatataatccaatacgtttctttttgagttgtttcatattacttcattcctttcaggagttattaaatgtgattgttcaacgatattgagtggactgtttttaaagtagattccttgtaattgtttagtttgagttattttgatgcaatcgataaaaggtgctatatcctgtaaagttattttattttttatgacatattttaatttgtcatcgatttggtaggttgaataagtagagtaattctcttcacttttacaactagcagataaacgtttgaacgtttttacatcaatacgattttgagctagcttttcgataagttgttcttgtgttagatgggtttctttatgcttcatttgttgctgttttaggactttgagtattgtgttattcaatcgtttatgaaacgattgttcttcaaaatactttttacaagtgtcgagcacttcactttcaagttctggtgcattgatacttttaaatgggcatgtacgataagcgtcattcatatttttaggacaaacatagtaacgtagagaatagttctctttttttatcgttaagtttgttaatgttgattgacagtaaggacatttgattcgtcgctttagcttatttctagaattggatcgattgagttgtttatgaatacgacgctcttgtgcttcttcaaatgtatcaatatcaataataggtggaacgatatcattaaacgtgccatatttattgatgacacgaccgcaatagttagggttcaagagaatatttctaacttgatagggcttacgaggaataaggttaggattactatccaaatgttgggaaatctttttgtagcctagaccttgtaagtaccagcgataaaccgatttaactgtatacgcttcttcttcttgtacaacaaaacaaccttttctataacgatagccaaacggagcatgagttgtgattagcttaccttgtttggctttttctctaatcccattttttgtttgttcgctgatattgtttgattccatttccgctagactcataagtatgttcaaacgaaagcaatcaaactctttagacaaatcaaaatatccatcgttaacactgatgattgtgatgtgatgctttttacaaatttcaaagaattgtatggcatttttcaaattacgatgtagtcggttcaagcgatagcaacacaatactttacattttccagacgtaattatttctaccattttttgataacctgaacgttttgtatgtcgacctgttttcttatcatcataaaacgccacattagaccatccatattgcttagcggtatccataatgagcgatttttgagtagctaagctttgttgtttgagtgtactttgacgtacataagcaatcgcttcttccatgttatacacctccaaaaagataatatatatttgtgagtaaattagaatgaaaggtccaacgtgcttttaacacgttggaccgtcgtgattagttatcgttctgtagctcttcaacgactaaatcagctagtaattcaatcaattcatccattttatctactcctgtacgatttcatctttaagttattaaaaatcaattaaatcatcgttctgttttttccatgattcaagtatctttttgttatccaagttaatttcagctttaataggttcagcatctttagttagaccaaaaatagacgcatattttgaatctagctttaaatggtaaaagacaagtgactgtttcttgccatagtcatctttaactgctcttttcgtagttattcgatcacggtcagattcgataaatcctttatcccttagtgcattcacaacattgttgacatcttggaaatgatgctctatcaacatattcttaaatacagatgcaatgattttaacttcgatataatcatcttttaaggcaattagtccatagttctcaatcatattctttaatgctgtatcatcagaaaacttaccacggttttgtgctacaaattgcgtaatgacttcaatcgctttatcagccagtgatcgttcagagactgtatgagcatgataatcaataaagtagtctcttattttagcgatatcaatatctgtagataaaacatgactaagaatcttggccgaagtagtgatagccgcatagcgcttaaacatacgaataccagtattatttgtttcatccttcaatttatctttaaaccaatgatgtaattcaaaaacggatcaaaaaacaaaaggccctagaaactaggagcacacttacacaagaacaattgatagataaattagctaaaggagccatcgatgcagaaacgttcagaaaacaatctcaatcgttacttcaacaatcaaaaccaacactatcaataaatgagcaacaaattcaaaggtcttttgaaaatgtaattcaacaacacttcacgttaagcatgttataccgatatattgatgaaattcatatttctaaaaacaaaagccttgtgggaatctatttcaaaaatgaaccgctaaacattgtaaaccaagctacgcaatcatcgattgcttaattaatgaaaggatgaaaaatatgaaagaaatgaaaagaaaatctgtaggatgttatgttagagtttcaacaatttctcaagacattgataaatttagtattaatggtcaaattacacaaataaaggaatattgccaacagggaaattatgaattgctatactgataattaaaagtacccccgatcgaacgaaaatatctttttgatgactattagtgataattgtaattttatccattaatagatgaatggattggaataatactaagcctattgctattccaccgagtaaaattacccacccgcctaattcaatactttccggaatcatttcaaaaaataataagccaattataaggcctgcacataatgaataaataaagccatgaccactttgaaaaccttccattatccaagcgagtccaccgccaacaccaattccaagtgcggaagctaacgcccgatcatccatactgcactcatcccactattcttaaaaaggctttgtttattttactctaaaaaatcaaaacaaaatatatgaattcatattaaatattatttgactctttattcagatggttatatcatatatatgaacatatattaatatatgtagataaatagtgattattatatataaggttttgaattgaaacctaaagtgagggaaggataatggaagaaaaaaaggaattagaggaagtaaataataaggacttagatgatgaaacattatttgtcgtatcgcaaacatttaaagcgttaggtgatcctacgagaatccggattctccatttgctcttttataaggagtattcggtaaacggtattgctgaaacgctacatcttagacaatcaacagtttcccatcaattgcggttcttgaaaaatttacggttagtaaaattccgaagggaaggcacaacattgttttattcccatgatgatgaacatactatgaatatgctaaaacaggcgatcgatcacgcctgtcatcactagtattccgtaaaccagtaatgagtgttaataataattcacgtcattaatattgttatatgatcatataaaaatattataggcatatgaaaagggggttttgcaatggatatggaaaacaaaaaaacagaatggaaagcgttgtatgatatctcgaaggaaagtgagatgggtgtagcagagagggttagtgaatacggttgataaattcataggatgtattattggatacaaaaagtttggatgtggtcaattaacgtcctggtttagtaaagcttactgcgaatatattggaacccgatatatcatatttatttagaaaggtgatagaaatgaaaaatattcaagagcaacaagcacacgaaagtcatagccacgatcatagtcatgatcatgatcacggaaaaatgccaattatttcatattttattggcttagtgttggctataattgggctttttttaagtgatgcaaatttattaatacaaaacatcttattttcaattgccacaatcacagccggctaccatgtaattattctcgaaggaattggagagacagttgaaaatactaaattaaagggaaaattcactcctaattctcatattctaatgggattagctgcaatcggggcttctctgatagggagtttttgggaaggaacccttttgatacttattttttccggcgctcattttcttgaagattacgctgaaggaaaaagtaaaagagaaattactaagctactcgaaatgaacccaacgacagctaaattaatcttacctgatggaaacacaaaaattgttgatgtcagtgaattaaaagttggagatcaactccaagtgctgaacggtgatcaagttccaattgatgggataattttatccggtactacctcaattgatgaatcttctattaatggagaaagtataccgaaagagaagtctaagggtgacgaagtttttggaagtacgattaatggaacaggtacttttactatggaagtcactaaggaaaacaaggatactgtattctctaaaattttacaattagttagtcaaaaccaagataatcaaacaaaagctgccagtatcattcaaaaattcgagcctaaatatgttaatatagttttaatcgcaataccattagtaatgttacttgctccttttctatttgattggacatggtcgcaaagtgtttacaggggattagtgcttttagtcgcagcttcaccgtgtgctttggcagcagctactgtatctgtaacattgtctacaacatctaacctagctaaaaaaggcgtgctttcaaaaggaagtacttacctatcacaattagcggatatagatgcaattgccttcgataaaacaggaacccttacgaacggagaacctaaagtaacaaattactatttcactcattctgtgaacgaagaaaatattattgatattatagtcgcccttgaaaaggaatccaatcacccactcgctaatgctattttagaaaaatttgaagttaaaaataaaatagacatcgaagttactaatcaaattggaaaaggtctgacaggagattataatggaaaaaattatcgtattggtaagcctacttcttttgaaagtgcttctgaagagtatacccagttcaatcatgattgggcatcagaaggaaagacggttgtatacgtagcagaaaatgaagaagttattgggattatagctctaatggatattccgaatgagcatgctaaagaaacaattaattactttaagaaacttggtatccacacgactttaattactggtgattcggaaatgacgggaaaagctgtaggcgaacaattgggaatagacgaagttatcgctaatgtaatgcctgaagataaatccagaattatagaagaacaaaaagaaaaatttggagttactgccatggttggagatggtgtgaacgatgcaccggcccttgttaatgctgatgttggtatagctatggggggcggtactgatgtggcagtagaagtatctgatttggttttaatgcagaacaatttatctaaattagtacagtctcataaaatttcctcaaatatgggtcgtgttattaggcaaaatattattttttcaatggcagttgttgcctttttagttgtcgttagtttgttaggattaactgatattacaatcagtgtaattgttcatgaaggaagtactttagttgttatactaaatggacttcgattattaagatctaaataatgaacgaatcgattgacatgaatgaactttgaagtgtggattctacaatgttcccataacattggacactaaaaaacagagcaatctaataaagatgttatgagtaaaaacaatgccttgcataccgtttttatcatacgggcggtcaaataagcaattaaagagcatgggaagcataaaatcataaacagtgacttaaggcagccagtttacattcaaaagttaaattgactgtattaaaagtttcgaaacgattaatatccgtatgggggcaagcgtcgagccaaagataatgccaggacagagcgtctttttccgttcttttaagtgggaaaggttttaccttctctatgccaagacagtcccagagctaaaagggccagttgctctttttgggcgtttgtgaacggtaatcgcaggaaggggattttcccaaccgcacctggttttccatcacaataaagttggcggcatttgttctgatattcttgggaatacttacactatgaatggccgtattaagtgatacaggagctgctattattgtaatactgaatgctctccgccttttgagggtaaaagaataaaagtaaggataactaggtaaagctgttcaatcaaaaattgaacagcttatttttcatcaaaatcaaaaacgtttattataatacctacacttgttctgttaaatgtaccgattttttaaccttattgtatcagtaatatcttgaaacgaagtaagcgactaaaatttctcttttatcatacgtttataaaaatacacttttaagaacggtttgaaaatttttgaaatagatcaaaataatcttctggttaaaaaacctgtagatatgatttctctcttaaatttttgtttgattagattagaccctaataagacgcattcaatatgtctacacgtgaatttagtctttgaaaatgtaaggaccattattattataaaaaccccagtataaaacgatacgctgaagcgtaccacaaataaaactaaaaaaatgaatattataatgatatattaacaattaaaagtttgaacttttttggcaatgatgttaaggcaaaaatgtattgaagaaagttaagagtttgggacataaagttctaggataagtgaaaaaagacaatttctattgaagtaatataaaaattgtcttttttataatttttttgattattttcagatcgttgagctgttacatttcttatattaagtgccatcagcacaaattctagttctcttttgtcttttttattcctcgaacggacattcgagtgaaatccaaaatggccttaataaatccaaaaaacaggctccacatcaactttttttgactgtagatggtttttgtttttgatttgaagtttatactttatacattggtgtttcagaggacattctgaacaatcatcacattcatataatttaaagtctcgtttaaaaactatatttatcatgactataggcatatcttttaaatcctagtcttttattattcggacaagtgaattcatcgttaatttcgtcataatccccattttgagtattaagaaatgtcacttttatattttttagttttatcttttataaacatttcataagttatgagtggcattcgattaaaatgatagaatgctttagagtctttattcatccttgactcaggttttgaatactatttttccatacaaatgtatatcgattggcatttgcctcaactttcgtatcatcaataaaaatggcttgatcatctctaagattttacttcaaacactgaatatgaaattgaataaataacgtttctaataaagcatcactttgggatttactctaaatcgattaatcgttttataagaatgctttagattttgtgacagccacaccattcggattctatcattgagcaatttttctatcttacgtcctgaaaacacagattgtgtgtaggtatatagaactacttttaacatcatttttggatagtatgatgttgcaccatgataatgtctgaattcatcgaattcagtctcgggaattgtttctacaatatcatttggggtgagtattatttgatgaaagctgaagataaaagttagaataaaattactaaatatagatttgacatctttataaattaagaatttgtcaaaataaagggaggcattctatagataatggtgaatgatgattttacgaatgagttatatcaacttatagttcctatggatgataaaataaaagaaaacattgatgaagtattatctttctacaatattaatttcaaaaaatatttatttagtatctcttcgttagtaacctttgaaaagtgtaataaattacgaggaagaattgctaaatatcctatttttacaattttatatgaaaaagtagaatatgatgaaaaagggattaagattataattaatagtaaaattcttaatgacatacaaacgcatgtgagtcatattcctaatactactgaaatttatttggttaactttaataacctaaacatttctgaaaaaacaaagaaagaattggttaaagaattaaataaaatgtttaacgaccagaaaaaaagaaagttagcccatcaagttgtaaatattatagagataataaaattggatagtcatggtaagtctctaattgatatcggctatttctatttaggtgctaaaaatagtgctttaaagtttcaaaaaatacatattcaagaaaaaatggactatcggggaaagtaatttcaaatcgatttgaattattaagagatatctatgatatagacatgaatcaatttcaaccctattttgataagaaagatagtaaaacaaaaagatcaaattatgaatttgatatggagcaattcgaatctataagtacattagtagatttgttagagaattacccacttcctaaaagtgaaaataatattaaacatttagaaagtattaagaaaaatatatttagtgataacgcagatgaatacgaaaagtattttaatgaaggtatagataaacttaaagaaggtgcatttgaattacaaaagcaattatataaccagtatccatttcaattacttatgcgatttcagtatgaattaactgaattaattaagcatattagtatgtattttaaacaagtaaatcagtcaaatatatatgaagctacgactttatacgaaaaattaaacaattttattttaagaagtctacttgagaataactcagaaaaagttaattttaaaaactttttgaatggagtaaaatttgaagaatcaacaatcaatgcagatgaggaaataattgttaaaaaatacaatgattttgtagataatattgagtttaataaagatgattttgataaactatatgatcaatatgaggtgtttaatagtataactaaattagttacattaaatgaagatgaaataaaatgggatcatatatttgtatataaatataaattaaaaacccctttcattgataaaataatcattgataacttgaaaaacattaaaagtcaatatattgatagtaaaactatgttggaaagagaatacaataaaaatggtaattttatttttgattttttaaatttagcaaaatgtaaagatgagaatgaattagatgaggaaatagatgagttactcttcaatcttgataatataggtaacagattactaaagaaagttgtattaggtcactctaaaaatataaaatattcaaacgcactattaaaagaaattcggagaatgataaaggttttaaatttatattcttcacaaaaaaatcaatccaacatggttgtcacttaccatcaattgcaaaagagcgataatgaatttgataatagaattaatttacttacaaaggaattagtttatctaagagtaaagaaagaactttttaataacagtaaatatactcataatgatgatagatagacatttaatttgaataatgaataagtttaagctaactcaattgagttagcttttttggcttccccctctttaatgaattttggggaagctaacgattagttctaaaaatgttgataaagtatatcttgtaacgttactaataaagatttcccgggcaatcaaacttgggggtgagtttaaaaatggaaagcaaagtagttgtaaaagtagaacatgataacgataaattacgagttgagctaaacgtttcgaaaagagatgactttaactatcaaatgcactttgcaataagtgaacgtgcatatgcattactaactgtaatgattggtagagcgttaatgttaccaagagggacagtattctcatgctatgaaaggttctgttgcaaagttgaatttatagtataattttaacaaaaaggagtcttctgtatgaactatttcagatataaacaatttaacaaggatgttatcactgtagccgttggctactatctaagatatgcattgagttatcatgatatatctgaaatattaagggaacgtggtgtaaacgttcatcattcaacggtctaccgttgggttcaagaatatgccccaattttatatcgaatttggaagaaaaagcataaaaaagcttattacaaatggcgtattgatgagacgtacatcaaaataaaaggaaaatggagctatttatatcgtgccattgatgcagagggacatacattagatatttggttgcgtaagcaacgagataatcattcagcatatgcgtttatcaaacgtctcattaaacaatttggtaaacctcaaaaggtaattacagatcaggcaccttcaacgaaggtagcaatggctaaagtaattaaagcttttaaacttaaacctgactgtcattgtacatcgaaatatctgaataacctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatattttttactttgcaacagaacctccaatatcattaaagcaacaccagaaactatgaaaacatcagcgagattaaagataggataatcaatgagccgaaaatcaaggctatctattacttctcctctaatcaagcggtcaaaaaagttccctaatgctccacctagcaacagtgataaggcaaaagagattcgtcggttattccttccttctgtttgtaaataataaatgatacccagtacaacaattaacgttacaataataaaaaaccagcgttgaccttgcaaaattccaaatgctgctcctttatttcgaagagaagtaatatggaatactccttcccataaaggaatggtttgacctatttccattcatttgacaatcgcagatttccatagttgatcaatgattaaaacgatgactgcaatgatatagtagatcctgattctcctcctccaatattcattattttaaagtataccatgatataacgaaaaaatgtatattttattgttgcaataaaacgttatattatttatactaatatatatacaaataataatttgaatgttattttatacactaattacagaggaggggttgttttgagtattacttccataaataaaaaagaacatgatttagatggtataaaatcaaggggaaaagatgcttgtgagacatttatatttgacccagaaaaggttgagcgccgtcagcaggaagtcggtcaaacggatggcttatcacaattgtttaaagtttttgcggatgatacacggttgaaaattgtttatgcacttacgcaagaagatgaaatgtgtgtctgtgatgtagcaacgattattggttctacgaatgctactgcttcacaccacctacgtatgttacgtaatatggggtttgccgagtaccataaaaaagggaagatagttttttataggctagtaagtaatcatgtgcatcaccttatgcaagaagcactacagttgaaggaaaagaagtaaatgtgatgagtgttgggaaaaattagttaaaatgttacaaacaactcagtcctccgttttacatgatggaatgcagttgctcaaaaataaaccataaaaattacctaagaaaagaccagtagataaaaaatcatctcaggagatttaagacctggtatccactagggcttaaatcttgttttttctctgaattgtaccctgatggtccaaattatttgtgaaatggaatgaattaaagtaaggagagtttgtaatgttctcatggactgtagtatctcttgtgttactcacaagtatagctaatcttgttggaggatttattgtcgtgagaaaagaatggtcccccaaagctcttacatatttaatggcttttagtgcaggttttcttttatccataggtattcttgatctaatgccagaaggattagaaaattcacctgaaaatggaatctatatattaataggatttttagtattattttcctttcaaagaatcctcacaactcattttcatttcggctatgaaacacatgaggataaactcagtaaaaaaacgggcggtttaggtgcctttatcggaatgaccattcatagtttctttgatggggtctccattgttgcgggattcgaagtaagcagtgaattaggtttccttgtgtttgttgctgtattacttcataaaataccagatggcttaaccatttcttcgattgttcttgtagtatttaatgacaggaaaaaggcgtttattgcctctgcagtactggctttggctactatatttggaggagctttagtatggctactcagtgacacagaatttgcagctgaagtacttggcgattcttttgcaagaattgctttgtccttttcagcaggtgtttttctatatgttgcagcaacggacttacttccagttgttaatcaatctgaaaatcgaaaaactggtctttacgttttgcttggtgtagccgttttctatatagcttcttggatcataggagttgttgggttggaatgaaaatattaaaggaatagccttgaataatcagttgaggaaattaaacagaaaagttgagtttagtcttagcaaaaaaagaaataaaatacaatcaaataatcatttgactatctatatgtgtggatatataataacaacagaacataacgtaatattcaaatgaatatcataatgagagatggccaagaatataggaattccacatgcattgattgataaaagatacattggttaattaagaaggaggttattaaatatgagtgaagcaacgaagccattagaggagaaatatgtctatcgtgttgatggattttcatgcgcaaactgtgcaggaaagtttgaaagaaatgttaaaaaaattccgggagtagaggatgcaaaagtaaattttggagcatccaaaatttccgtttacggagaagcaacggttgaagaattagaaaaagcaggtgcctttgagaaccttaaagtggctccagaaaaacctaggcggcaggctccgcaagaggttaaaaaagacaaaaatatatatcgtgttgaagggttttcatgcgcaaactgtgcaggaaagtttgaaagaaatgttaaaaaaattccgggagtagaggatgcaaaagtaaattttggagcatccaaaatttccgtttacggagaagcaacgattgaagaattagaaaaagcaggtgcttttgagaatcttaaagtggcctctgaaaaacctgtacggcaagctacacaagagattaaccaggaaaaagaggatgaaaaagaagaaaaagtgccattctataaaaagcacagtactttactctattcgaccttattgattgttttcggatatctttcagtctttgttaatggggatgagaacattgttactacattattatttgtagcatcgatgttaataggaggattatcgctttttaaagttggtttccaaaacttgttacgctttgaatttgacatgaaaacacttatgactgttgcagttataggtggtgcgattataggagaatgggcagaagtttctgttgttgtcatactctttgcaattagtgaagctctagaacgattttctatggataaagcgagacaatcaattcgatcattaatggatattgcacctaaagaggcacttgttagacgaaaaggtcaagaaatgatggttcatgtagatgatattgcagttggagatattatgattgtcaaacctggtcaaaagattgccatggacgggatggttgtaagtgggtactctgctgttaatcaagctgccattacaggtgagtcagtccctgttgaaaaagcggttgatgatgaagtatttgcaggtactctaaatgaagaaggattacttgaagtagaaataactaaattagtagaagatacaaccatttcaaaaattattcatcttgtagaggaagcacagggagaacgagcaccatctcaagcctttgtagaaaaatttgcgaaatattatactccgatcatcatgatcatagctgcgttagttgctgtagttccaccattattctttggggctagttgggaaacatgggtttatcaaggtttagctgttttagtcgttggttgtccatgtgcgttggttatttccaccccgatttcgattgtttcagcaattggaaatgcagcgaaaaagggtgtcctaataaaaggtggcgtttatttagaagaaatgggtgccttaaaggccattgctttcgataaaacaggtactttgacaaaaggtgtccctgttgtaactgacttcaatgtgttgaacaaacaggtagatgaaaatgaaatgctatccatcattactgcattagagtatcgttctcagcaccctcttgcatcagcaatcatgaaaagagcagaagaggcgaatatttcttattcagatgttgtgatagatgacttctcttccattacaggaaaaggaattaagggtactgtagacggaacgacctattatattggaagtccaaaactatttaaggaactgtcaaactctagttttgataagaatttagagaaaaaagttgcaactcttcaaaatcaagggaaaactgctatggttgttggaactgacaaagaaatactagctattattgcagtagcagatgaagtacgtgaatcaagtaaagaagtcattcaaaaactacatcaacttggcatcaaaaatacaattatgcttaccggtgataataaaggtactgcgaatgccattggaagtcatgtcggagtaaaagaagttcaggctgaattaatgcctcaggataaattggattatattaaacaattgaaatcggaatacaacaacgtagctatgattggcgatggtgtcaatgatgcacctgcattggcagcttccactgttggaattgcaatgggtggagctggtactgatacagcattagaaacggcggacgttgcactaatgggagatgatttaagaaaacttccgtttacggtaaaactaagtcgaaaagcgctaaacataataaaagctaacattacttttgcaattgctattaaatttattgccttattattggttattccaggttggttaacactctggattgcgattctttcagatatgggtgcaactcttcttgtagcgttgaacagtttacgacttatgcgagtgaaagattaagagtaaaaagagtgtgggacataaagtcccaggataagtaaaaaaagacaatttctattgaaataatatagaaattgtcttttttattatttttttgattattttcagctcgttgagctgttacttttcttatattaagtgccattagcacaaatcctaattctcttttggctttatctatccctcgaacggacattctagtgaaacccaaaatagccttcataaatccaaaaacaggctccacatcaatttttctttgactgtagatggtttttgtttttggttctgaaagctttttattaatttgggctttaaaatattcccaattataatttttcattatttttttatttgtttttgaattgaagttcatacattgttgtttcagagggcattctgaacaatcatcacattcatataatttaaagtctcgtttaaaaccatatttatcatgacgataggcatatcttttaaatcctagtcttttattattcggacaaatgaattcatcgttaatttcgtcataatcccaattttgagtattaaagatgtcacttttatattttttagttttatcttttataaacattccataagttatgagtggcgttcgattaaaattatctataattgccatataattggcttcgctaccataacctgcgtcagctacaatatattcaggtaaatgaccgtaggtctcttgtattgtatttaaaaaaggtatcattgttcttgtatcagtcggattttgatacacatcataaaataaaacaaattgagaatttgtcgctatttgtaaattataccccggtttaagttgtccatttttcatatgatctgttttagaataactatttctatcctttaaaatagatttttggacttcgtatttatgctttcgctgagagtaatcattaatttgctttttgtattttttaattttagttcttttgagccgtatttgttttcttattttagtacatttttcattatcgatatgttggtttaaatcttcgatttctttatctaaatgactaccaatcaaatctatttcttcttttgttaagtcattatcatgatcttttttaatctcaggtatgattttatttatcaccaattcatgatagagtgctttagagttttcattcattcttgactcatggttttgaatactctttttccatacaaatgtatatcgattggcatttgcctcaactttcgtaccatcaataaaaatggcctgatcatcaataagattttgtttcaaacactgactatgaaattgaataaataatgattctaataaagcatctactttgggatttactctaaatcgattaatcgttttataagaaggcttttgattttgtgatagccacatcattcggatactatcattgagtaatttttctatcttacgtcctgaaaacacagattgggtgtaggcatataaaactacttttaacatcatttttggatggtatgatgttgcacctcgatgatgtctgaattcatcgaattcagtctcgggaattgtttctacaatatcatttacatgtcgtgaaatatcatttgtggggataagaactgaagtttccattggtagagtaagttgagtcatgttataatctttatacataaggcacctcgttaatttagtttagtgatgtttattaaattatacgaaagggccttatttttttaaagtattttaatgtaaaattacatatgaatacaaagtattttggcgagactcttgagggaacaggacaagctgaagactacaggctgaagctgtcccctaagaaagcgagccaacaatacgaagtattgtaaataaagaagccagtaaatgaatttatgaaaactcatttactggctattttgataggaattatgtcccaggcccttttaatcaaaatgtagattattttttataaattgtctaataagagaagaaaaataggtgaaaataagaaaaaggttagatacatattacacaactatattcttgagtacaaatagtaattatagttacttatcaaaggaggaatctaggatgactggagaagtgaaaaaatatgtagaccttttagtgataggagcagggtctggaggttatgtggcggcaatacgtgctgcccagttaggaaaaaaggtggtactggttgataaggcggagttaggaggagtttgccttaatcgagggtgtatcccttcaaaagctcttattagtgcatctgaacgagtcaaacacattaaacatgccaatacaatggggcttaaggtttcgggtgaggttcaagttgatatgccagaagtagtaaagtggaaggacggaatcgtgaacaagctaacagatgggattcgcaccttgttaaaaggaaatggtgtagaggtcatcagtggggaagcctatctcactgaagcacatattgcaaaaataaaaatagaggacgaggaacagattttttcctacaaagatttaattcttgcaataggttctttgccagtcgagttaaaaagtatgccatttgatcaaaaaagaatcatttcttcaacggaggcattgcaactacaggaagtacccaatcatttagttgtggtaggtggtggttatatcggtttagagttaggtactgcttatgcaaagtttggggcaaaagtgaccatccttgagggatcggatacaattttgtccgggacagatcctatcctaacaaaaacagtaaaacgtcatttaaaggaaattggaatcactgttataacggatgcccttgttcaaggtggagaaaatacaggcgatgaagttaacgtccatgttcaagttgatggaaaggaagaaattattcaatgtgattattgcttagtttctattgggagaaaaccaaacacaggtaaaattggtctggaaaatatcggagtagtattagacgaccaaggatttattaagataaataacaaatgtcaaacaaacattgaacatgtttatgcaataggggattgtgctggtggtgacctccttgctcataaagctagttatgaagcaaagatagcagccgaagtgatcagtggtcaaaatagtgtgattgattttcaagcgatgccttttgttatcttcagcgatcctgaagtagcctatacaggtttaacagagaaggaagcaaaggaaaaaggatacgaaacagtatccagtcgttttccattccaagcaaatgctagggcgttatctgtctcggatgcagatggctttgtacaagttgtagccgaaaagaatacgaaaagagtgttaggagtacagatggttggaccagaggtatcctctctgattgcagaggctgtttttgcgattgaggctggggcaaatgcggaagatcttagccttacgattcatgctcatcctaccttaccagagccacttatggaagcagcagaaggtgtgatgggacatgctacacatatgttgaataaaaagcaataatgtgaatgcaggccaacctgcactttttcataagtgcaggttgaatttttattaatggatggagactcatgcacattttgtattgatagatgtattactgtataaaattcctgatggtatggattaatcactattcatgaatttctaatgaactgggttaatacaaattatgaaatatatcaaatcgtttttctgactgtttatgaacgatgaaggagttgatataaatgaatttaattttaactattctctctgctttaggttcttttatagtaaccaatgtcgatgatatttttgtcttgatgttactgttctctcaagcaaggtcacaagctaaaacgggtaacagtggaatagttagcatggagtcgaaaggaaaccgtatttatccaagagatatagtcattggccaatatcttggttttgcacttttagttttaatcagtcttttagcaacatttggagtaacactcattcctgatcagtgggtaggtttgctaggattaataccgatatatctaggagtaaaactctttataaagggtgaagatgaggatgaaggtaacattctttccaacttaaacaaatttaataagttctatttaagtgtagcctttatcacattcgccaatggcggagacaatattggaatttatgttccattcttttctaccttaaataacaaccaactgataattacagttgttactttctttataatggttgctgtctggtgtttaattggctatcgtctagcaaggttcagatatgtttctgaaacacttgagaaatacggccgctggattatcccaattgtgtttataggattaggaatatatattatggtggagaacgaaactttccgcgccattttgagtttagtgaattaaaaatttctcaaaagtacctctgatttacagtaggactttgatatttagagtaaatatttatcctctgacaaataattctatggatacgctcatctatattttgcttagcgtacaaaatttccgaaatgttggctgtaccccaataagcaattagaatattatcaaaattgttaattaagaatagccctcgaaaaaatcgggggctttgcttttggcataagacggccaaaacaaagccgaaccaaaaggagtgaatgtgatgtgcataacgcacacacatcacattcacattaaaactaaattcaaaatggagggaaagtgaagattcactttccctttattttcatttttagaatttatttttttgtgttgaaacgctcacacgtacaatcaaagattgtacttttaacaagcctactcaatcttacaaggggcaataaaactttttaggttctgttgcaaagtaaaaaatatagctaaccactaatttatcatgtcagtgttcgcttaacttgctagcatgatgctaatttcgtggcatggcgaaaatccgtagatctgaagagacctgcggttctttttatatagagcgtaaatacattcaataccttttaaagtattctttgctgtattgatactttgataccttgtctttcttactttaatatgacggtgatcttgctcaatgaggttattcagatatttcgatgtacaatgacagtcaggtttaagtttaaaagctttaattactttagccattgctaccttcgttgaaggcgcctgatctgtaattaccttttgaggtttaccaaattgtttaatgagacgtttgataaacgcatatgctgaatgattatctcgttgcttacgcaaccaaatatctaatgtatgtccctctgcatcaatggcacgatataaatagctccattttccttttattttgatgtacgtctcatcaatacgccatttgtaataagcttttttatgctttttcttccaaatttgatacaaaattggggcatattcttgaacccaacggtagaccgttgaatgatgaacgtttacaccacgttcccttaatatttcagatatatcatgataactcaatgcatatcttagatagtagccaacggctacagtgataacatccttgttaaattgtttatatctgaaatagttcatacagaagactcctttttgttaaaattatactataaattcaactttgcaacagaaccatttgcgatacaacgaggcgacattagccgttaatttgcttaactgtagaagatttaaaagaaccccttgccatatatttaacaatgttgtccgggataacaacacagtgaatccaccgtttagttaagatgatggattttttcaaactgtggatgaggtacagagtttcattagataaaaacagggaaaacttattaaagagtgatttctgtgaggaggataatgatgacaaagaaatacgatcttattgtgattggtacaggttctgcggggtcaatcacagcagcaaaatgtaataaagccggctggaatgtagctatggtagatgacaggccgtttggaggcacgtgtgcactacgtggatgtgacccaaaaaaggttctacatggtgctgccgagcttattgattggaacaaacggatggttaagaatggagtaccgtctgaagtctccatcaactggaaagatcttatgaattttaaaagaacctttacagatgatgtcccggaaaaaaaagaggaagcattgaataaacagggaatcgatacgtatcatggcaatgcatctttcgtgagtgaggacaaacttgaagtgaataaagaagtgctcgaggggagtcatttcttgatcgctagtggtgcgaaacctacccctttacccataaaaggagaagaacatcttacttatagtgatgagtttttggagttggatgaattaccacaaagaatcgtctttgttggtggtgggtatatttcgtttgaatttgctcatatcgcggcacgtgctggatcagaagttcatattatccatcgaggtcaaagacctttggagaattttgatatagatcttgtagatatccttttagaaaagtcgaaagagattggcatacaggtacatcttcaacattccgttgaatccattgaaaaagaacaaggaaagtttcatgtgtacgctcgaaaaaaagaggatataacgcggtttgaagcagacattgttattcatggtgctggacgggtccctgccttagatatgaatcttgaaaaagggaatatagaaaggaaaaaacatggtgtccatgttaatgagtatttgcaaagtgtaagtaacccgaatgtctatgcagctggagatgctgcagcaacggatggcttgcccctcacacctgtagccagtgcagattctcatgtcgtagcatctaatttattgaaagggaacagcaaaaaaattgaatatcccgtgattccatctgctgtatttaccgtacctaaaatggcatcggtaggtatgagcgaggaggaagccaaaaactctggccggaatattaaagtaaagcagaaaaacatctccgactggtttacgtataaacggacaaatgaggactttgctgcgtttaaagtgctaattgacgaggatagtgatcaagttgtcggtgctcatttgattagtaatgaagccgatgaactaattaaccactttgcaacagccattcgctttggaatttcaaccaaagaattgaaacaaatgatatttgcctatccaacggcagcttcggacattgcacacatgttgtaagtttgcgttttgtgagatgtttataaaccactttatcgtctaatcttgttatatgatcttagaatcaaaaagaaaataacttttttgacagccacatttttcaacggaagaatgtggcttcttggcgtgaagagcagatatctttattcttaaacttctaaaaaagcttatgtgaacacttgataaataaggttttttatctgacctattttataagattattctataaaagaaaaaataatgtatgacttgaccgtgtactatagtacagggtttatactttttattgaggtgacaatatggagatgaaaatcagtgaattggctaaagcgtgtgatgtgaataaagaaaccgttcggtattacgagcggaaaggattgatagccgggcctcccagaaacgaatcagggtatcgaatatattcagaggaaacagcagatcgggtacggtttattaaacgaatgaaggaattggatttctcgctaaaggaaatccacctgttgtttggtgtggttgatcaagatggggagagatgtaaagatatgtacgcctttaccgttcaaaaaaccaaagaaatcgagcggaaagtgcaggatttgttacgaatccaacggttattagaggaattaaaagaaaagtgtccagatgaaaaggcgatatatacctgtcctattattgaaacgttaatgggagggcctgataaataaatgggggaagatttgaatgaaaaaacgcatttccttcaccgctataatgacggtgttgttaatcggtttaacagcttgtggagcagaatctgataccgcgaatgaatcaaaggttcaagacattcaaggaaatccagtcagcctacctaatgagaaacccacactcatttattttatggcaacctggtgtccatcttgtatatacaatgaggaaatctttaaggaaattcatcaactaaacccgaacgatgttcaattgatcacagttagtttagaccctaacacagatacaaaagaaagctttgcgaaatttaaacaggattatggcggggattggccccatgttttaaagataggagtggaattatgagtttttcgtttctcttcattctgaccgctggaatggttgcggcctttaatccttgtggtatcgccttgcttccttcttatatctcctatttaattggaggcgaaacaaaggatcattcgttccgctatgcaatttttaaaggattagggcttggtggagcgatgaccacggggtttttaacgatttttgtattggctggtttgttgataggaggattgggaagcgcactaacagggatttttccgattctttcattggttatgggtatactcattgctttattggggttgggcatgctatttgggaagcatttgccgattaaaataggatcttttcaagtcaagccagggaaatggtctatctatttttacggaatagcctatgccgtgacatcacttggttgtaccttgccagcctttatgttggtggtctctgcatcgctgaatgacaatagcgtaacggccgtgatcatcaagttcatcatctactcccttggtatgggaattgtggtgacagcgatcacgatggtctcattgatttcacgacaattggtacagaagtttctgcacaactatatgggttctatccaaaaaatagcagctgttgtgattttcctctccggtttgtacatggcttattactggtatttcggttctggtggcatttgtacattttaaaatccaaccgatcaggtgtaacaaaaaatagatgaaagatgtaaaaaagctaaacgaacaaatttcaagttaggctataggccatgtatgtaccttaaccaaagaataaaatttttttagaaagagtggtgagaatgatggatgagaatcgctcgaaaggtaaccgctggggtgtttgggcctttttcggaatattattggttcctttattggttcctttactttgttgcgcaggaccgattctccttgttgcattagggagcacaggtattggtgccctctttgctggtgcgacaggaaattggtggttgaccggcatttttgcagcattggccattgttatgattgctctgatcctcagcaagttgttgaagaacaaatacaattctcctgaaggcaatggaaaaacaaaaaataaaacggattgctgtacgcctccagagagcgtggatcggaaacatgagacaagataaatatgagataaatgtacagaactgtctatcaaactttaaatagaaagagggattcaaatgactcaaaattcatataaaatacccattcaaggcatgacatgcacaggctgtgaagaacatgtaaccgaagcattggaacaagccggagctaaagatgtttcggccgatttccgacgcggtgaggccatttttgaactcagcgatgatcagatcgaaaaggctaagcaaaatatttcggcagccggctatcaacccggagaggaagaaagccagccctctgaaaacagtgtagatttcaatcgggatggcgattacgatcttctgattattggttccggcggtgcggcgttttctgcagctatcaaggccaatgaaaacggggcgaaagtggccatggtagaacggggaaccgtcggggggacctgcgttaacatcggttgtgtaccgtcaaaaaccatgcttcgtgccggtgaaataaacggtctcgcccaaaacaatccgtttaccggacttcaaacgagtaccggtgctgcggaccttgcccaattgaccgaacaaaaagatggattagtcagtcaaatgcgtcaagaaaaatatatagacttgatcgaagaatatggatttgatctcattcgtggcgaggcctcgtttatcgacgataagacgatacaagtgaatggacaaaacatcacgtctaaaagctttttaatcgcaacgggggcttctccggctgttccggaaatcccgggaatgaatgaggttgattatttaacaagtacatccgcactcgaattaaaagaggttccacaacgattggcagtgatcggttctggctatatcgcagcggaattaggtcaaatgtttcacaacctcggaacagaagtgactctcatgcaaagaagcgagcgtctgtttaaaacctacgatcctgaaatttccgaagccatcgatgaatccttaactgagcaaggacttaacctgatcactggggtcacttatcaaaaggttgagcaaaacggtaagtcgacaagcatttatattgaagtgaacggtcaagaacaagtcatcgaagccgatcaagtcctcgtggcaacaggaagaaagccgaacacagagactttaaaccttgaatcagcaggtgtgaaaacagggaaaaaaggcgaagtgctgaccaatgaatatttgcaaacgtcgaataaccgaatatatgccgcgggcgatgtgaccctcggtccgcaattcgtttatgttgcagcttatgaaggcgggattgtggcaaataatgcgttgggtctagcgaaacgcaaaatcgatcttcgctttgttcccggcgtaaccttcaccaatccatcgatcgccacagtcggcttgactgaacaacaggcaaaagaaaaaggttacgatgtcaaaacatcggtccttccgttggacgctgtaccgcgggccttagtcaatcacgaaacaacaggggtctataaacttgtagtcaacgcccaaacccagaaattgatcggggcgcacattgtgagtgaaaatgctggagatgtgatttatgcggcaacgttagcggttcaatttggattgaccattgaagaccttacggatagctttgcgccttatttaacgatggctgaaggattaaagcttgcagccttgacgtttgataaagacgtatcgaaattatcttgttgtgcaggctaatggttccttttgattctaatgctatgttgcaggaggcacttaaaacaatgaagtggagaaaatataaggaggaataagcttatgaaaaatatttcagaattctcagcccaacttaatcaaacttttgatcaaggggaagccgtctctatggagtggttattccgtccgttgctaaaaatgctggcggagggcgatccagtccccgttgaggacatcgcggcggagaccgggaagcccgtcgaggaagttaagcaagtcctacagactctacctagtgtggaacttgatgagcagggccgtgtcgtcggttatggcctcacactggtccctaccccccatcgcttcgaggttgatgggaagcaactatatgcatggtgcgcccttgacacacttatgttcccagcactcatcggccggacggtccacatcgcttcgccttgtcacggcaccggtaagtccgtacggttgacggtggaaccggaccgcgttgtaagcgtcgagccttcaacagccgttgtctcgattgttacaccagatgaaatggcctcggttcggtcggccttctgtaacaacgttcactttttcagttcaccgagtgcagcccaagactggcttaaccaacaccctgagtcgagcgttttgcccgttgaagatgcctttgaactgggtcgccatttgggagcgcgttatgaggagtcaggacctactaatgggtcctgttgtaacatttaatcttaggcaattacgcctgcgtcaatcttggccttacgatgatgtcctgtaaccgaagctctatctagtgaaaccaattttatgggaaaccaaataaataaaaagatcaattgcttgtgcctatatcggggagttaataaagcgagaaagtggattcttgaatataggagtaaataaggtgccaaaatggcaccttatttaaaatatgtaacgccaaggtctgccgtattatattgcattggaaaaagaaaaccttctgtataaggagatgttatatgatatttggtaaactgttcggcagtttgggcgtagaaagcttttgctccttgaacatcacgtttgccagttacaatgtcatttaacaagtttaaagataaaaaattcatcgcttcctcatgacactttgctgatgcctcacctgtagttcgatctaagtagacacttccatcaaaagcggcaatcgcatcatataagtgtacgggcactttataatcgatggtctgcttaaggaaatcaaggtgaggggtagggaagttatgagggactggatccttatatactatcgtgcgtttccatggcccgctattgtaccaaattaatcgtctcattgttgcttcctggggatatccatacttactaattatggtttttgccccttccaacggtggtccctgccaataggacaaaatgtgatttaaatgactatccattgccatacctcctaaacatttgcctaaaagcattgtatgcccaataaatgaattttaggtaaatgtatatttaatgatcggcagattgcagtaaataacgaatgcctgcaatgcaattaacatgggatttaacactggacagtaatttttttgctgggattttgctatcagatagcttttcttagtgatgataggatatgttgaccaaattggccgtgagattggaggggtggtttttatggttagttgttgttcaatcgatccatacactgaattcgaagtcattattattggtgccggggtgccggaggtttctcttgcctttcatttgacttcatacggcataaggacggcacttgcggatgaacagaacgtggataacagattaaaggactttttaagaacttttcaagaattgattgagcgcgattcgggtatgccgtgaacgttacaggcaataaaagaagatatgcacctcatcctttttgacggtaccattacccttgaaggtgaacatgctgtccgtattgatgaacacaaaattcggggggatcgtatggtcgtaccatttgatacgtttaaacaaataggaaaacctagaaaccaaaacgacatccattttaaaaacgatccttttcaaagagttacgaatgatttatggatcacgaatccctcattgtcagaagtggatcaagaacaaatatggcagactggagactatttttcaaaacaccttcagaacattgaataaatgaagggttctgttgcaaagtaaaaaaatatagctaaccactaatttatcatgtcagtgttcgcttaacttgctagcatgatgctaatttcgtggcatggcgaaaatccgtagatctgaagagacctgcggttctttttatatagagcgtaaatacattcaataccttttaaagtattctttgctgtattgatactttgataccttgtctttcttactttaatatgacggtgatcttgctcaatgaggttattcagatatttcgatgtacaatgacagtcaggtttaagtttaaaagctttaattactttagccattgctaccttcgttgaaggtgcctgatctgtaattaccttttgaggtttaccaaattgtttaatgagacgtttgataaacgcatatgctgaatgattatctcgttgcttacgcaaccaaatatctaatgtatgtccctctgcatcaatggcacgatataaatagctccattttccttttattttgatgtacgtctcatcaatacgccatttgtaataagcttttttatgctttttcttccaaattcgatataaaattggggcatattcttgaacccaacggtagaccgttgaatgatgaacgtttacaccacgttcccttaatatttcagatatatcacgataactcaatgcatatcttagatagtagccaacggctacagtgataacatccttgttaaattgtttatatctgaaatagttcatacagaaggctcctttttgttaaaattatactataaattcaactttgcaacagaaccttattgcatatctttttagccatatcactgatagggtgaagcatatcataaatgatgcggttttt

SEQ ID NO: 89 - DNA, 3050 bp, Staphylococcus aureus
  source 1..3050: mol_type = genomic DNA
tcgtgccattgatgcagagggacatacattagatatttggttgcgtaagcaacgagataatcattcagcatatgcgtttatcaaacgtctcattaaacaatttggtaaacctcaaaaggtaattacagatcaggcaccttcaacgaaggtagcaatggctaaagtaattaaagcttttaaacttaaacctgactgtcattgtacatcgaaatatctgaataacctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccgaaaataatctcttcaatttatttttatatgaatcctgtgactcaatgattgtaatatctaaagatttcagttcatcatagacaatgttcttttcaacattttttatagcaaattgattaaataaattctctaatttctcccgtttgatttcactaccatagattatattatcattgatatagtcaatgaataatgacaaattatcactcataacagtcccaacccctttattttgatagactaattatcttcatcattgtaaaacaaattacaccctttaaatttaactcaacttaaatatcgacaaattaaaaaacaataaaattacttgaatattattcataatatattaacaactttattatactgctctttatatataaaatcattaataattaaacaagccttaaaatatttaacttttttgtgattattacacattatcttatctgctctttatcaccataaaaatagaaaaaacaagattcctaaagaatataggaatcttgtttcagactgtggacaaactgattttttatcagttagcttatttagaaagttttatttaaattacagtttctatttttattagatcacaattttattttagctcttgttcaagtaatcatttttcgccaaaaactttatactgaatagcttctacattaaatactttgtcaatgagatcatctacatctttaaattcagaataatttgcatatggatctataaaataaaattgtggttctttaccggaaacattaaatattcttaatattaaatatttctgcttatattctttcatagcaaacatttcatttagcgacataaaaaatggttcctcaatactagaagatgtagatgttttaatttcaataaatttttctacagctttatctgtatttgttggatcaaaagctactaaatcatagccatgaccgtgttgagagcctggattatcatttaaaatattcctaaactgttctttcttatcttcgtctattttattatcaattagctcattaaagtaatttagcgctaatttttctccaactttaccggttaatttattctctttatttgatttttcaatttctgaatcatttttagtagtctttgatacaccttttttatattttggaattattcctttaggtgcttccacttccttgagtgtcttatctttttgtgctgttctaatttcttcaatttcgctgtcttcctgtatttcgtctatgctattgaccaagctatcataggatgtttttgtaacttttgaagctaattcattaaatagttctaaaaatttctttaaatcctctagcatatcttcttctgtgaatccttcattcaaatcataatatttgaatcttattgatccatgagaatatcctgatggataatcattttttaaatcataagatgaatctttattttctgcgtaataaaatcttccagtattaaattcatttgatgtaatatatttattgagttcggaagataaagttaatgctctttgttttgcagcatttttatcccgcggaaacatatcacttatctttgaccatccttgattcaaagataagtatatgccttctccttccggatgaaaaagatataccaaataatatccatcctttgtttcttttgttatattctcatcatatattgaaatccaaggaactttactatagttcccagtagcaaccttccctacaactgaatatttatcttcttttatatgcacttttaactgcttgggtaacttatcatggactaaagttttatatagatcacctttatcccaatcagattttttaactacattattggtacgtttctctttaattaatttaaggacctgcataaagttgtctatcatttgaaattccctcctattataaaatatattatgtctcattttcttcaatatgtacttatttatattttaccgtaatttactatatttagttgcagaaagaattttctcaaagctagaactttgcttcactataagtattcagtataaagaatatttcgctattatttacttgaaatgaaagactgcggaggctaactatgtcaaaaatcatgaacctcattacttatgataagcttctcctcgcataatcttaaatgctctgtacacttgttcaattaacacaacccgcatcatttgatgtgggaatgtcattttgctgaatgatagtgcgtagttactgcgttgtaagacgtccttgtgcaggccgtttgatccgccaatgacgaaaacaaagtcgctttgcccttgggtcatgcgttggttcaattcttgggccaatccttcggaagatagcatctttccttgtatttctaatgtaatgactgtggattgtggtttgattttggctagtattcgttggccttctttttcttttacttgctcaatttctttgtcactcatattttctggtgctttttcgtctggaacttctatgatgtctatcttggtgtatgggcctaaacgtttttcatattctgctatggcttgcttccaatatttctcttttagtttccctacagctaaaatggtgattttcatgtc

SEQ ID NO: 90 - DNA, 3050 bp, Staphylococcus aureus
  source 1..3050: mol_type = genomic DNA
acctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccgaaaataatctcttcaatttatttttatatgaatcctgtgactcaatgattgtaatatctaaagatttcagttcatcatagacaatgttcttttcaacattttttatagcaaattgattaaataaattctctaatttctcccgtttgatttcactaccatagattatattatcattgatatagtcaatgaataatgacaaattatcactcataacagtcccaacccctttcttttgatagactaattatcttcatcattgtaaaacaaattacaccctttaaatttaactcaacttaaatatcgacaaattaaaaaacaataaaattacttgaatattattcataatatattaacaactttattatactgctctttatatataaaatcattaataattaaacaagccttaaaatatttaacttttttgtgattattacacattatcttatctgctctttatcaccataaaaatagaaaaaacaagattcctaaagaatataggaatcttgtttcagactgtggacaaactgattttttatcagttagcttatttagaaagttttatttaaattacagtttctatttttattagatcacaattttattttagctcttgttcaagtaatcatttttcgccaaaaactttatactgaatagcttctacattaaatactttgtcaatgagatcatctacatctttaaattcagaataatttgcatatggatctataaaataaaattgtggttctttaccggaaacattaaatattcttaatattaaatatttctgcttatattctttcatagcaaacatttcatttagcgacataaaaaatggttcctcaatactagaagatgtagatgttttaatttcaataaatttttctacagctttatctgtatttgttggatcaaaagctactaaatcatagccatgaccgtgttgagagcctggattatcatttaaaatattcctaaactgttctttcttatcttcgtctattttattatcaattagctcattaaagtaatttagcgctaatttttctccaactttaccggttaatttattctctttatttgatttttcaatttctgaatcatttttagtagtctttgatacaccttttttatattttggaattattcctttaggtgcttccacttccttgagtgtcttatctttttgtgctgttctaatttcttcaatttcgctgtcttcctgtatttcgtctatgctattgaccaagctatcataggatgtttttgtaacttttgaagctaattcattaaatagttctaaaaatttctttaaatcctctagcatatcttcttctgtgaatccttcattcaaatcataatatttgaatcttattgatccatgagaatatcctgatggataatcattttttaaatcataagatgaatctttattttctgcgtaataaaatcttccagtattaaattcatttgatgtaatatatttattgagttcggaagataaagttaatgctctttgttttgcagcatttttatcccgcggaaacatatcacttatctttgaccatccttgattcaaagataagtatatgccttctccttccggatgaaaaagatataccaaataatatccatcctttgtttcttttgttatattctcatcatatattgaaatccaaggaactttactatagttcccagtagcaaccttccctacaactgaatatttatcttcttttatatgcacttttaactgcttgggtaacttatcatggactaaagttttatatagatcacctttatcccaatcagattttttaactacattattggtacgtttctctttaattaatttaaggacctgcataaagttgtctatcatttgaaattccctcctattataaaatatattatgtctcattttcttcaatatgtacttatttatattttaccgtaatttactatatttagttgcagaaagaattttctcaaagctagaactttgcttcactataagtattcagtataaagaatatttcgctattatttacttgaaatgaaagactgcggaggctaactatgtcaaaaatcatgaacctcattacttatgataagcttcttaaaaacataacagcaattcacataaacctcatatgttctgatacattcaaaatccctttatgaagcggctgaaaaaaccgcatcatttatgatatgcttctccacgcataatcttaaatgctctatacacttgctcaattaacacaacccgcatcatttgatgtgggaatgtcattttgctgaatgatagtgcgtagttactgcgttgtaagacgtccttgtgcaggccgtttgatccgccaatgacgaatacaaagtcgctttgcccttgggtcatgcgttggttcaattcttgggccaatccttcggaagatagcatctttccttgtatttctaatgtaatgactgtggattgtggtttaattttggctagtattcgttggccttctttttcttttacttgctcaatttctttgtcgctcatattttctggtgctttttcgtctggaacttctatgatgtctatcttggtgtatgggcctaaacgtttttcatattctgctatggcttgcttccaatatttctcttttagtttccctacagctaaaatggtgattttcatgtcgtttggtcctccaaattgttatcaactttccagttatccacaagttattaacttgttcacactgttccctcttattataccaatattttttgcagtttttgatattttcctgacattta

SEQ ID NO: 91 - DNA, 2996 bp, Staphylococcus aureus
  source 1..2996: mol_type = genomic DNA
atgaaacgagccattggttatttgcgccaaagtacaacgaaacaacaatcactcccagctcaaaagcaagcaatagaattattagctccaaagcacaatattcaaaatatccaatacattagtgataagcaatcaggcagaacagataatcgaacaggctatcaacaagtcaccgaacgcatccaacaaagacaatgtgacgtattatgttgttatcgcttgaatcgacttcatcgcaacttgaaaaatgcattaaaactcatgaaactctgtcaaaaatatcatgttcatattctaagtgttcatgatggctattttgatatggataaagcgtttgatcgcctaaaactcaatatattcatgagtctggctgaacttgaatccgataatattggagaacaagtcaaaaatggacttagagaaaaggcaaaacaaggtaaactcataacgacccatgcgcctttcggttatcactatcaaaatggtactttcatcattaataatgatgaatcacctaccgtcaaagctgtattcaattattatcttcaaggatatggctacaagaagattgcacaatatttagaagacgataataaacttattacccgcaagccttatcaggtacgaaatataattatgaacccaaattattgtggtcgtgtcatcaatcaatatggtcaatataacaatatggtaccacctattgtttcggcaacgaaatatgaacatgctcaagcaatccgtaataagaagcaacttcactgtataccttcagagaatcagctgaaacaaaagatcaaatgtccttgttgtgactcaacactgacaaatatgacaataagaaaaaaacatacattgcgatattatatttgtcctaaaaatatgaatgaatctcgctttgtctgttcattcaaaggaataaatgcacaaaaattagaagttcaagtcttagctacatgtcagaacttctttcaaaaccaacagctctattcaaaaattaataatgcaattcatcaacgcctcaaaaaacaaagagtgatagaagctaaaagtacgctaactcaagaacaactgatagataaacttgccaaaggtatgattgatgctgaatcattcagaaaacagactcatttgatgaatcaaaagcacaaaaccatatcctccataagtgataatcagttacaaacatcactacaaaaggttatacagaaaagtttcacgttaaacatgctgcatccctatattgatgaaattcgcattacaaaaaataaagcccttgttgggatctatttcaaaaatgaaccattgaacattgtgaaccaaacctcgcaatcatcgattgcttaatcagaaaggatgaaaaaatcatgcaacaactcaaacaaaaacgtgtcggtatctatgttcgtgtatcaacggaaatccaaagtactgaaggctatagtatcgatggacaaatcaatcaaattcgagaatattgtgatttcaataactttgttgttgtagatgtatacgcggatagaggtatctctggaaaatctatgaaccgaccagaactacaacgtttgttaaaagatgcgaacgaaggtcagattgattctgttatggtctacaaaacaaaccgactagcacgtaacacttctgacttactcaaaattgttgaagaccttcatcgtcaaaatgtcgaattcttcagcttatctgagcgtatggaagtcaatacaagcagtggtaaattgatgctacaaattctagcgagtttttcagaatttgaaagaaataatattgtcgaaaatgtattcatgggtcaaacccgacgcgctcaagaaggctattatcaaggcaatttgccgctgggctatgacaaaataccggatagcaagcatgaactcatgataaaccaacatgaagcgaatattgtcaaatatatatttgagtcatatgctaaaggccacggatatcgtaaaattgcgaatgcactcaatcacaaaggatacgtgactaaaaaaggaaagcctttcagtattggttcagtgacctatatcttatctaatccattctatgttggtaaaattcaattcgcaaagtacaaagattggaatgaaaagcgtcgtaaagggctgaatgataaaccaataatagctgaaggtaagcattcccctattattattcaagacttatgggataaagtccaattacgtaaaaaacaagtcagtcaaaaacctcaagtccacggtaaaggaactaatctattaacaggtatcgttcattgtccacaatgtggtgcaccaatggcagctagtaacacaacgaacacattgaaagatggtaccaagaagcgaatacgttattattcttgcagtaacttccgaaacaaaggctcaaaagtatgttctgcgaatagcgttagagctgatgtgattgagaaatacgtcatggatcaaatactcgaaattgtcaaaagtgataaagtcattaaccaagtcttagaacgtgtcaatcaagaaaataaagtcgatattggtgcattgaaccacgatatcgcttataaacaacaacaatacgatgaagtcagcgggaaactccataatttagttaaaaccattgaagataatccggacctaacatctgcattgaaagcaactattcatcaatatgaaacacaactcaatgacattacaaatcaaatgaatcaactcaaacagcaacaaaatcaagagaaactatcttatgatacgaaacaaatcgctgccctattacaacgaatatttcaaaatatagaatcaatggataaagcacaactcaaagcattatatcttacagtcattgaccgtattgatattcgtaaagacggtaatcataaaaaacagttctacgttacactaaaactcaataatgaaattattaaacaacttttcaataatacccctctcgacgaagtgctcctcagcacttcgtctttatttttgcctcaaacgctctttcttcaaatctaa

SEQ ID NO: 92 - DNA, 2153 bp, Staphylococcus aureus
  source 1..2153: mol_type = genomic DNA
ctgtagggaaactaaaagagaaatactggaagcaagccatagcagaatatgaaaaacgtttaggcccatacaccaagatagacatcatagaagttccagacgaaaaagcaccagaaaatatgagcgacaaagaaatcgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtaccacaaatgatgcggttttttatccagttttttgtttaatgaacaaggtaaattacgagataatatttgaagaaaacaataaagtagagatggatttccatatcctctttagtagcggtttttatctgtaaggtttattaataattaaataaataggcgggatagttatatatagcttattaatgaaagaatatgattattaatttagtattatattttaatattaaaaagaagatatgaaataattattcataccttccaccttacaataattagttttcaatcgaatattaagattattagtagtcttaaaagttaagacttccttatattaatgacctaatttattatttgcctcatgaattatctttttatttctttgatatgtcccaaaccacatcgtgatatacactacaataaatattatgatgaaactaataatattctcaaagttcagatggaaccaacctgctagaatagcgagtgggaagaataggattatcatcaatataaagtgaactacagtctgttttgttatactccaatcggtatctgtaaatatcaaattaccataagtaaacaaaattccaatcaatgcccatagtgctacacatattagcataataaccgcttcattaaagttttcataataaattttacccataaaagaatctggatatagtggtacatatttatcccttgaaaaaaataagtgaagtaatgacagaaatcataagaccagtgaacgcacctttttgaacagcgtggaataattttttcatagtgagatggaccattccatttgtttctaacttcaagtgatcaatgtaatttagattgataatttctgattttgaaatacgcacgaatattgaaccgacaagctcttcaatttggtaaagtcgctgataaagttttaaagctttattattcattgttatcgcatacctgtttatcttctactatgaactgtgcaatttgttctagatcaattgggtaaacatgatggttctgttgcaaagtaaaaaaatatagctaaccactaatttatcatgtcagtgttcgcttaacttgctagcatgatgctaatttcgtggcatggcgaaaatccgtagatctgatgagacctgcggttctttttatatagagcgtaaatacattcaataccttttaaagtattctttgctgtattgatactttgataccttgtctttcttactttaatatgacggtgatcttgctcaatgaggttattcaaatatttcgatgtacaatgacagtcaggtttaagtttaaaagctttaattactttagccattgctaccttcgttgaaggtgcctgatctgtaattaccttttgaggtttaccaaattgtttaatgagacgtttaataaacgcatatgctgaatgattatctcgttgcttacgcaaccaaatatctaatgtatgtccctctgcatcaatggcacgatataaatagctccattttccttttattttgatgtacgtctcatcaatacgccatttgtaataagcttttttatgctttttcttccaaatttgatataaaattggggcatattcttgaacccaacggtagaccgttgaatgatgaacgtttacaccacgtccccttaatatttcagatatatcacgataactcaatgcatatcttagatagtagccaacggctacagtgat

SEQ ID NO: 93 - DNA, 1696 bp, Staphylococcus aureus
  source 1..1696: mol_type = genomic DNA
aaagagaaatattggaagcaagccatagcagaatatgaaaaacgtttaggcccatacaccaagatagacatcatagaagttccagacgaaaaagcaccagaaaatatgagtgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgttttcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcatatcataaatgatgcggttatttcagccgtaattttataatataaagcagagtttattaaattttaatgattactttttattaagaattaattctagttgatatattataatgtgaaacacaaaataataatttgtaattgttagtttataggcatctgtatttggaattttttgtagactatttaaaaaatagtgtatataagtattgagttcatgtattaactgtcttttttcatcgttcatcaagtataaggatgtagagatttgttggataatttcttcggatgtttttaaaattatcattaaattagatggtatctgatcttgagttttgtttttagtgtatgtatattttaaaaaatttttgattgttgttatttgactctcttttaatttgacaccctcatcaataaatgtgttaaatatatcttcatttgtacttaaatcatcaaaatttgccaacaaatatttgaacgtctctaaatcattatgtttgagttccgttttgctattccataattccaaaccatttggtagaaagcccaagctgtgattttgatctccccatatagctgaatttaaatcagtgagttgattaattttttcaacacagaaatgtaattttggaatgaggaatcgaagttgttcttctacttgctgtacttttcttttgttttcaataaaatttctacaccatactgttatcaaaccgccaattattgtgcacaatcctccaatgattgtagataaaattgacaatatattacacacctttcttagaggtttattaacatctatttttgaatttaaaattattactttggtagcgttataacctatttaacagattagagaaaaattgaatgatcgattgaagaatttccaaaataccgtcccatatgcgttgaaggagatttctattttcttctgtattcaaatctttggctttatcctttgctttattcaataaatcatctgagtttttttcaatattttttaatacatctttggcattttgtttaaatactttaggatcggaagttagggcattagagtttgccacattaatcatattattattaatcatttgaatttgattatctgataatatctctgataacctacgctcatcgaggactttattaacagtgtcttcaacttgttgttgtgtgatttgtttatcttgattttgtttaatatctgcaagttgttctttaatatctgctatagaagcatttaaagcttcatctgaatacccat

SEQ ID NO: 94 - DNA, 991 bp, Staphylococcus aureus
  source 1..991: mol_type = genomic DNA
accattttagctgtagggaaactaaaagagaaatactggaagcaagccatagcagaatatgaaaaacgtttaggcccatacaccaagatagacatcatagaagttccagacgaaaaagcaccagaaaatatgaactacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcgtatcataagtgatggtaaaaaatatgagtaagtagatgaagagtgaaaatcagattaattaataataatgtatcaaatttaaataaaggggtttttaagtatgaatttaagaggtcatgaaaatagacttaaatttcatgcgaaatatgatgtgacacctatatcacatttaaaattattagaaggtcaaaagaaagacggtgaaggcggcatactgacagatagctattactgtttttcatacagcttaaaaggtaattctaaaaaagttttaggtacgtttaattgtggttatcatattgctgaagatttactaaaattatcaaatcaagataaattacctttatttaacccgtttaaagtaattaatgaaggtaatcaattgcagggcgtaacgaataaaggtaatttaaatattaataggcaaagaaaacagtataatgaagtggctttacagctttcaaatgctattaatttaatcataatttgttatgaggataatattaaagaaccactttcaacgataaaatac

SEQ ID NO: 95 - DNA, 1108 bp, Staphylococcus aureus
  source 1..1108: mol_type = genomic DNA
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaagcgtaccacaaataaaactaaaaaatatgagaaaattattaaattagctcaaatctttgaagaataaaaagtgaatattaagtttgataatttaggtacaagtaaagattaagaatttccattatttaatacatggtgtgtaaatcgacttctttttgtattagatgtttgcagtaagcgatgtaaagaagatgctaataaatatgtgaggaatgattacgatactagataagcggctaatgaaattttttaaagtacatatatagacatatttttcatttagtaaaattttgaatttcactttgctaagactagtgtctagaaatttataatgatttattaacacctatttgaaacttaagtataataaatgattcggattttatttttaataaagacaaacttgaacgtagcaaagtagtttttatgataaataataagttttaataatgtgacgcttttatataagcacattattatgaacaatgtgaattgagcatctacaattacattaataaatatataaatgatgatttaaattcacatatatttataatacacatactatatgaaagttttgattatccgaataaatgctaaaattaataaaataattaaaggaatcatacttattatacgtatacgtttagct

SEQ ID NO: 96 - DNA, 1530 bp, Staphylococcus aureus
  source 1..1530: mol_type = genomic DNA
ttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaagcatatcataaatgatgcggttttttcagccgcttcataaaggggggtgatcatatcggaacgtatgaggtttatgagaattgctgctatgtttttatgaagcgtatcataaatgatgcagtttttgataattttttctttatcagagattttactaaaaatcccctcaaagtttgtttttttcaacttcaactttgaagggaataaataaggaacttatttatatttatcctttatctcattaatatctatttttttattaataatattataaatattaaattctttagaaaagtcactatcactcttattcttcatactaaacgttattaatctaataatatcagctactatttctttaaattctattgcatcttcttttttataagtagcgcctgtatgaacaattttatttctcataccatagtaatctttcatatatttttttacacaatttttaatttcattagaattatccaaatctagattatcaattgtctttaataaatgatcattaacaacattagcatacccacatccaagcttcttttttatctcttcatcacttaaattttcatctaatttataatatctttctaaaaaatttgtgataaaaacttctaatgcagtctgaatttgtacaattgctaaattatagtcagatttataaaaagaacgttcaccttttctcatagccaaaacataaatattgctaggatgattattgaaaatattataattttttttaatatttaataaatcactttttttgatagatgaatactgatcttcttctatctttccaggcatgtcaatcatgaaaatactcatctcttttatatttccatctatagtatatattatataatatggaatacttaatatatcccctaatgatagctggtatatattatgatactgatatttaacgctaataattttaataagattatttagacaattaaattgcttattaaaaattttcgttagactattacttttctttgattccctagaagtagaatttgatttcaattttttaaactgattgtgcttgattattgaagttatttcaacata

SEQ ID NO: 97 - DNA, 1256 bp, Staphylococcus aureus; identical to SEQ ID NO: 37
  source 1..1256: mol_type = genomic DNA